(19) United States
(12) Patent Application Publication
(10) Pub. No.: US 2024/0141369 A1
(43) Pub. Date: May 2, 2024

(54) DOMESTICATION OF A LEGUME PLANT

(71) Applicant: BETTERSEEDS LTD, Givat Chen (IL)

(72) Inventors: Ido MARGALIT, Gan Yavne (IL); Tal SHERMAN, Rehovot (IL); Shira COREM, Sdei Hemed (IL)

(73) Assignee: BETTERSEEDS LTD, Givat Chen (IL)

(21) Appl. No.: 18/279,761

(22) PCT Filed: Mar. 2, 2022
(86) PCT No.: PCT/IL2022/050227

§ 371 Date: Sep. 1, 2023

Related U.S. Application Data
(60) Provisional application No. 63/155,340, filed on Mar. 2, 2021.
(60) Provisional application No. 63/252,182, filed on Oct. 5, 2021.

(51) Int. Cl. C12N 15/82; A01H 6/54; C12N 9/22

(52) U.S. Cl. CPC C12N 15/8262; A01H 6/54; C12N 9/22; C12N 15/8205; C12N 15/8213; C12N 2310/20

(57) ABSTRACT

The present disclosure relates to conferring desirable agronomic traits in Legume plants. More particularly, the current invention pertains to producing Legume plants with improved traits by manipulating genes controlling plant architecture.

### FIELD OF THE INVENTION

The present disclosure relates to conferring desirable agronomic traits in Legumes plants. More particularly, the current invention pertains to producing Legumes plants with improved traits by manipulating genes controlling plant architecture.

### BACKGROUND OF THE INVENTION

One of the most important determinants of crop productivity is plant architecture. For many crops, artificial selection for modified shoot architectures provided critical steps towards improving yield, followed by improvements enabling large-scale field production. A prominent example is tomato, in which the discovery of a mutation in the antiflorigen-encoding self-pruning gene (sp), led to determinate plants that provided a burst of flowering and synchronized fruit ripening, permitting mechanical harvesting.

The publication of Li et al (2018), nature biotechnology, “Domestication of wild tomato is accelerated by genome editing”, teach the assembly of a set of six gRNAs to edit four genes (SlCLV3, SlWUS, SP and SP5G), into one construct. The construct was transformed into four S. pimpinellifolium accessions, all of which are resistant to bacterial spot disease, and two of which are salt tolerant. Small indels and large insertions have been identified in the targeted regulatory regions of SlCLV3 and SlWUS in T0 and their T1 mutant plants. It was reported in this publication that although SP and SP5G are crucial for improving the harvest index, the limited allelic variation has hampered efforts to optimize this trait. It was further reported that locule number was not increased in T0 and T1 plants with large insertions and inversions in the targeted SlCLV3 promoter region. One explanation for this finding is that the targeted region of the SlCLV3 promoter may not be essential for regulating SlCLV3 transcription. Alternatively, it was suggested that disruption of regions (gRNA-5) flanking the CArG element downstream of SlWUS may have decreased its transcription and counteracted the effects of mutation of SlCLV3, owing to a negative feedback loop of CLV3-WUS in controlling stem cell proliferation.

The publication of Zsögön et al (2018), nature biotechnology, “De novo domestication of wild tomato using genome editing”, discloses a devised CRISPR-Cas9 genome engineering strategy to combine agronomically desirable traits with useful traits presented in Solanum pimpinellifolium wild lines. The four edited genes were SELF-PRUNING (SP), OVATE (O), FRUIT WEIGHT 2.2 (FW2.2) and LYCOPENE BETA CYCLASE (CycB).

Lemmon et al (2018), Nature Plants, “Rapid improvement of domestication traits in an orphan crop by genome editing”, describes the usage of CRISPR-Cas9 to mutate orthologues of tomato domestication and improvement genes that control plant architecture, flower production and fruit size in the orphan Solanaceae crop ‘groundcherry’ (Physalis pruinosa).

In open field crops, such as Legumes and Cocoa, it is essential for enabling sustainable agriculture, to be able to harvest the plants mechanically, instead of manual labor. Furthermore, resistance to plant diseases, such as fungi and viruses are also essential in order to maintain profitability and sustainability for open field crops.

Certain open field crops, including soybean, have been domesticated through conventional breeding to express determinate characteristics that enable easier harvesting via machinery.

Certain legumes, such as peanuts and/or cowpea, due to lack of genetic diversity or investment in breeding, do not exhibit this trait.

In view of the above, there is still a long felt and unmet need to manipulate Legume plant architecture in a rapid and efficient way to increase yield and reduce production costs.

### SUMMARY OF THE INVENTION

It is one object of the present invention to disclose a modified Legume plant exhibiting at least one improved domestication trait as compared to a corresponding control Legume plant, wherein said modified plant comprises a mutated SELF PRUNING (SP) gene selected from Cowpea (Vigna unguiculata) SP gene or Peanut (Arachis hypogaea) SP gene.

It is another object of the present invention to disclose the modified Legume plant as defined in any of the above, wherein said Cowpea (Vigna unguiculata) SP gene is selected from VuSP1-VuSP7 comprising a nucleic acid sequence with at least 75% sequence identity to a sequence selected from SEQ ID NO:1, SEQ ID NO:113, SEQ ID NO:239, SEQ ID NO:340, SEQ ID NO:464, SEQ ID NO:616 and SEQ ID NO:796, respectively, or a functional variant thereof and any combination thereof.

It is another object of the present invention to disclose the modified Legume plant as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or a functional variant thereof and any combination thereof.

It is another object of the present invention to disclose the modified Legume plant as defined in any of the above, wherein said mutation is introduced using mutagenesis, small interfering RNA (siRNA), microRNA (miRNA), artificial miRNA (amiRNA), DNA introgression, endonucleases or any combination thereof.

It is another object of the present invention to disclose the modified Legume plant as defined in any of the above, wherein said mutation is introduced using targeted genome modification.

It is another object of the present invention to disclose the modified Legume plant as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is another object of the present invention to disclose the modified Legume plant as defined in any of the above, wherein said Cas gene is selected from the group consisting of Cas9, Cas12, Cas13, Cas14, CasX, CasY, Csn1, Cpf1 and any combination thereof.

It is another object of the present invention to disclose the modified Legume plant as defined in any of the above, wherein the mutated SP gene is a CRISPR/Cas9-induced heritable mutated allele.

It is another object of the present invention to disclose the modified Legume plant as defined in any of the above, wherein said mutation is a missense mutation, nonsense mutation, insertion, deletion, indel, substitution or duplication.

It is another object of the present invention to disclose the modified Legume plant as defined in any of the above, wherein the insertion or the deletion produces a gene comprising a frameshift.

It is another object of the present invention to disclose the modified Legume plant as defined in any of the above, wherein said plant is homozygous for said at least one mutated SP gene.

It is another object of the present invention to disclose the modified Legume plant as defined in any of the above, wherein said mutation is in the coding region of said gene, a mutation in the regulatory region of said gene, or an epigenetic factor.

It is another object of the present invention to disclose the modified Legume plant as defined in any of the above, wherein said mutation is a silencing mutation, a knockdown mutation, a knockout mutation, a loss of function mutation or any combination thereof.

It is another object of the present invention to disclose the modified Legume plant as defined in any of the above, wherein said mutation is generated in planta.

It is another object of the present invention to disclose the modified Legume plant as defined in any of the above, wherein said mutation is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for said at least one Peanut SP gene, and any combination thereof.

It is another object of the present invention to disclose the modified Legume plant as defined in any of the above, wherein said mutation in said at least one Cowpea and at least one Peanut SP gene is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof.

It is another object of the present invention to disclose the modified Legume plant as defined in any of the above, wherein said gRNA sequence comprises a 3′ NGG Protospacer Adjacent Motif (PAM).

It is another object of the present invention to disclose the modified Legume plant as defined in any of the above, wherein said construct is introduced into the plant cells via Agrobacterium infiltration, virus-based plasmids for delivery of genome editing molecules, or mechanical insertion such as polyethylene glycol (PEG) mediated DNA transformation, electroporation or gene gun biolistics.

It is another object of the present invention to disclose the modified Legume plant as defined in any of the above, wherein said mutation confers reduced expression of said at least one SP gene.

It is another object of the present invention to disclose the modified Legume plant as defined in any of the above, wherein said modified plant has decreased expression levels of said SP gene.

It is another object of the present invention to disclose the modified Legume plant as defined in any of the above, wherein the sequence of said expressed SP gene is selected from the group consisting of: at least 75% identity to any one of Cowpea polypeptide SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the modified Legume plant as defined in any of the above, wherein said SP gene encodes a polypeptide sequence selected from the group consisting of: at least 75% identity to any one of Cowpea SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the modified Legume plant as defined in any of the above, wherein said modified plant is semi-determinant.

It is another object of the present invention to disclose the modified Legume plant as defined in any of the above, wherein said modified plant has determinant growth habit.

It is another object of the present invention to disclose the modified Legume plant as defined in any of the above, wherein said modified plant flowers earlier than a corresponding control Legume plant lacking said mutated SP gene.

It is another object of the present invention to disclose the modified Legume plant as defined in any of the above, wherein said modified plant exhibits improved earliness as compared to a corresponding control Legume plant lacking said mutated SP gene.

It is another object of the present invention to disclose the modified Legume plant as defined in any of the above, wherein said modified plant exhibits suppressed and/or similar sympodial shoot termination as compared to a corresponding control Legume plant lacking said mutated SP gene.

It is another object of the present invention to disclose the modified Legume plant as defined in any of the above, wherein said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is another object of the present invention to disclose a modified Legume plant, plant part, plant tissue or plant cell as defined in any of the above, wherein said plant does not comprise a transgene.

It is another object of the present invention to disclose a plant part, plant cell, plant pod or plant seed of a modified Legume plant as defined in any of the above.

It is another object of the present invention to disclose a tissue culture of regenerable cells, protoplasts or callus obtained from the modified Legume plant as defined in any of the above.

It is another object of the present invention to disclose a method for producing a modified Legume plant exhibiting at least one improved domestication trait compared with a corresponding control Legume, said method comprises steps of genetically modifying at least one Legume SELF PRUNING (SP) gene selected from Cowpea (Vigna unguiculata) SP gene or Peanut (Arachis hypogaea) SP gene, the resultant mutated SP gene has reduced expression level.

It is another object of the present invention to disclose the method as defined in any of the above, wherein said method comprises steps of genetically modifying the at least one Legume SP gene using targeted genome editing introducing a loss of function mutation in the at least one Cowpea (Vigna unguiculata) SP gene or Peanut (Arachis hypogaea) SP gene.

It is another object of the present invention to disclose the method as defined in any of the above, wherein said Cowpea (Vigna unguiculata) SP gene is selected from VuSP1-VuSP7 comprising a nucleic acid sequence with at least 75% sequence identity to a sequence selected from SEQ ID NO:1, SEQ ID NO:113, SEQ ID NO:239, SEQ ID NO:340, SEQ ID NO:464, SEQ ID NO:616 and SEQ ID NO:796, respectively, or a functional variant thereof and any combination thereof.

It is another object of the present invention to disclose the method as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

It is another object of the present invention to disclose the method as defined in any of the above, wherein said method comprises steps of:
a. identifying at least one Legume SP gene in a predetermined Legume plant;
b. synthetizing at least one guide RNA (gRNA) comprising a nucleotide sequence complementary to said at least one identified Legume SP gene;
c. transforming the predetermined Legume plant cells with a construct comprising (a) Cas nucleotide sequence operably linked to said at least one gRNA, or (b) a ribonucleoprotein (RNP) complex comprising Cas protein and said at least one gRNA;
d. screening the genome of said transformed predetermined Legume plant cells for induced targeted loss of function mutation in said at least one Legume SP gene;
e. regenerating Legume plants carrying said loss of function mutation in at least one of said Legume SP gene; and
f. screening said regenerated plants for a Legume plant with improved domestication trait.

It is another object of the present invention to disclose the method as defined in any of the above, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said Legume SP gene.

It is another object of the present invention to disclose the method as defined in any of the above, wherein said SP gene is selected from the group consisting of Cowpea SP genes comprising a sequence having at least 75% identity to a sequence selected from SEQ ID NO:1, SEQ ID NO:113, SEQ ID NO:239, SEQ ID NO:340, SEQ ID NO:464, SEQ ID NO:616 and SEQ ID NO:796, or Peanut SP gene comprising a sequence having at least 75% identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

It is another object of the present invention to disclose the method as defined in any of the above, wherein said mutation is introduced using mutagenesis, small interfering RNA (siRNA), microRNA (miRNA), artificial miRNA (amiRNA), DNA introgression, endonucleases or any combination thereof.

It is another object of the present invention to disclose the method as defined in any of the above, wherein said mutation is introduced using targeted genome modification.

It is another object of the present invention to disclose the method as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

It is another object of the present invention to disclose the method as defined in any of the above, wherein said Cas gene is selected from the group consisting of Cas9, Cas12, Cas13, Cas14, CasX, CasY, Csn1, Cpf1 and any combination thereof.

It is another object of the present invention to disclose the method as defined in any of the above, wherein the mutated SP gene is a CRISPR/Cas9-induced heritable mutated allele.

It is another object of the present invention to disclose the method as defined in any of the above, wherein said mutation is a missense mutation, nonsense mutation, insertion, deletion, indel, substitution or duplication.

It is another object of the present invention to disclose the method as defined in any of the above, wherein the insertion or the deletion produces a gene comprising a frameshift.

It is another object of the present invention to disclose the method as defined in any of the above, wherein said plant is homozygous for said at least one Legume SP gene.

It is another object of the present invention to disclose the method as defined in any of the above, wherein said mutation is in the coding region of said gene, a mutation in the regulatory region of said gene, or an epigenetic factor.

It is another object of the present invention to disclose the method as defined in any of the above, wherein said mutation is a silencing mutation, a knockdown mutation, a knockout mutation, a loss of function mutation or any combination thereof.

It is another object of the present invention to disclose the method as defined in any of the above, wherein said mutation is generated in planta.

It is another object of the present invention to disclose the method as defined in any of the above, wherein said mutation is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof.

It is another object of the present invention to disclose the method as defined in any of the above, wherein said mutation in said Cowpea and Peanut SP genes is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively.

It is another object of the present invention to disclose the method as defined in any of the above, wherein said gRNA sequence comprises a 3′ NGG Protospacer Adjacent Motif (PAM).

It is another object of the present invention to disclose the method as defined in any of the above, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

It is another object of the present invention to disclose the method as defined in any of the above, wherein said modified plant has decreased expression levels of at least one of said Legume SELF PRUNING (SP) gene.

It is another object of the present invention to disclose the method as defined in any of the above, wherein said mutation confers reduced expression of said at least one SP gene.

It is another object of the present invention to disclose the method as defined in any of the above, wherein the sequence of said expressed Legume SELF PRUNING (SP) gene is selected from the group consisting of: at least 75% identity to any one of Cowpea polypeptide SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the method as defined in any of the above, wherein the Legume SELF PRUNING (SP) gene encodes a polypeptide sequence selected from the group consisting of: at least 75% identity to any one of Cowpea polypeptide SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

It is another object of the present invention to disclose the method as defined in any of the above, wherein said modified plant is semi-determinant.

It is another object of the present invention to disclose the method as defined in any of the above, wherein said modified plant has determinant growth habit.

It is another object of the present invention to disclose the method as defined in any of the above, wherein said modified plant flowers earlier than a corresponding control Legume plant lacking said mutated SP gene.

It is another object of the present invention to disclose the method as defined in any of the above, wherein said modified plant exhibits improved earliness as compared to a corresponding control Legume plant lacking said mutated SP gene.

It is another object of the present invention to disclose the method as defined in any of the above, wherein said modified plant exhibits suppressed sympodial shoot termination as compared to a corresponding control Legume plant lacking said mutated SP gene.

It is another object of the present invention to disclose the method as defined in any of the above, wherein said modified plant exhibits similar sympodial shoot termination as compared to a corresponding control Legume plant lacking said mutated SP gene.

It is another object of the present invention to disclose the method as defined in any of the above, wherein said modified plant exhibits suppressed or reduced day-length sensitivity as compared to a corresponding control Legume plant lacking said mutated SP gene.

It is another object of the present invention to disclose a modified Legume plant, plant part or plant cell produced by the method as defined in any of the above, wherein said modified plant does not comprise a transgene.

It is another object of the present invention to disclose a plant part, plant cell, plant pod or plant seed of a modified Legume plant produced by the method as defined in any of the above.

It is another object of the present invention to disclose a tissue culture of regenerable cells, protoplasts or callus obtained from the modified Legume plant produced by the method as defined in any of the above.

It is another object of the present invention to disclose the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

It is another object of the present invention to disclose an isolated polynucleotide sequence comprising at least 75% identity to a Legume SELF PRUNING (SP) sequence selected from the group consisting of SEQ ID NO:1, SEQ ID NO:113, SEQ ID NO:239, SEQ ID NO:340, SEQ ID NO:464, SEQ ID NO:616, SEQ ID NO:796, SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928.

It is another object of the present invention to disclose an isolated polypeptide sequence comprising at least 75% identity to a Legume SELF PRUNING (SP) sequence selected from the group consisting of SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929.

It is another object of the present invention to disclose an isolated nucleotide sequence comprising at least 75% sequence identity to a Legume SELF PRUNING (SP)-targeted gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795, SEQ ID NO:798-1024, SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051.

It is another object of the present invention to disclose harvestable parts of a modified Legume plant as defined in any of the above, wherein said harvestable parts are preferably shoot biomass and/or seeds.

It is another object of the present invention to disclose products derived from a modified comprising at least 75% sequence identity to plant as defined in any of the above, and/or from harvestable parts of a modified Legume plant as defined in any of the above.

It is another object of the present invention to disclose use of a nucleic acid encoding a polypeptide comprising at least 75% sequence identity to the sequence as defined in SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, in enhancing yield and/or domestication, in Legume plants, relative to control plants.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The present invention provides a modified Legume plant exhibiting at least one improved domestication trait compared with a corresponding control or wild type Legume, wherein said modified Legume plant is selected from a modified Cowpea plant comprising at least one mutated Cowpea (Vigna unguiculata) SP gene, and a modified Peanut plant comprising at least one mutated Peanut (Arachis hypogaea) SP gene.

According to further aspects, the present invention provides a modified Legume plant exhibiting at least one improved domestication trait as compared to a corresponding control Legume plant, wherein said modified plant comprises a mutated SELF PRUNING (SP) gene selected from Cowpea (Vigna unguiculata) SP gene or Peanut (Arachis hypogaea) SP gene.

According to one embodiment of the present invention, the Cowpea (Vigna unguiculata) SP gene is selected from VuSP1-VuSP7 comprising a nucleic acid sequence with at least 75% sequence identity to a sequence selected from SEQ ID NO:1, SEQ ID NO:113, SEQ ID NO:239, SEQ ID NO:340, SEQ ID NO:464, SEQ ID NO:616 and SEQ ID NO:796, respectively, or a functional variant thereof and any combination thereof.

According to a further embodiment of the present invention, the Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or a functional variant thereof and any combination thereof.

The present invention further provides methods for producing the aforementioned modified Legume plant using genome editing or other genome modification techniques.

The solution proposed by the current invention is using genome editing such as the CRISPR/Cas system in order to create cultivated Legume plants with improved yield and more specifically with determinate growth habit. Breeding using genome editing allows a precise and significantly shorter breeding process in order to achieve these goals with a much higher success rate. Thus genome editing, has the potential to generate improved varieties faster and at a lower cost.

It is further noted that using genome editing is considered as non GMO by the Israeli regulator, and in the US, the USDA has already classified a dozen of genome edited plants as non-regulated and non GMO (https://www.usda.gov/media/press-releases/2018/03/28/secretary-perdue-issues-usda-statement-plant-breeding-innovation).

Legal limitations and outdated breeding techniques significantly hamper the efforts of generating new and improved Legume varieties adapted for intensive and advanced agriculture.

The present invention provides Legume plants with improved domestication traits such as plant architecture and plant habit adaptation. The current invention discloses the generation of non-transgenic Legume plants with improved yield traits, using the genome editing technology, e.g., the CRISPR/Cas9 highly precise tool. The generated mutations can be introduced into elite or locally adapted Legume lines rapidly, with relatively minimal effort and investment.

Genome editing is an efficient and useful tool for increasing crop productivity, and there is particular interest in advancing manipulation of domestication genes in Legumes wild species, which often have undesirable characteristics.

Genome-editing technologies, such as the Clustered Regularly Interspaced Short Palindromic Repeats (CRISPR)-CRISPR-associated protein-9 nuclease (Cas9) (CRISPR-Cas9) provide opportunities to address these deficiencies, with the aims of increasing quality and yield, improve adaptation and expand geographical ranges of cultivation.

A major obstacle for CRISPR-Cas9 plant genome editing is lack of efficient tissue culture and transformation methodologies. The present invention achieves these aims and surprisingly provides transformed and regenerated Legume plants with modified desirable domestication genes.

To that end, guide RNAs (gRNAs) were designed for each of the target genes identified in Legumes to induce mutations in SP and genes through genome editing.

As used herein the term “about” denotes ±25% of the defined amount or measure or value.

As used herein the term “similar” denotes a correspondence or resemblance range of about ±20%, particularly ±15%, more particularly about ±10% and even more particularly about ±5%.

As used herein the term “corresponding” generally means similar, analogous, like, alike, akin, parallel, identical, resembling or comparable. In further aspects it means having or participating in the same relationship (such as type or species, kind, degree, position, correspondence, or function). It further means related or accompanying. In some embodiments of the present invention refers to plants of the same Legume species or strain or variety or to sibling plant, or one or more individuals having one or both parents in common.

A “plant” as used herein refers to any plant at any stage of development, particularly a seed plant. The term “plant” includes the whole plant or any parts or derivatives thereof, such as plant cells, seeds, plant protoplasts, plant cell tissue culture from which tomato plants can be regenerated, plant callus or calli, meristematic cells, microspores, embryos, immature embryos, pollen, ovules, anthers, fruit, flowers, leaves, cotyledons, pistil, seeds, seed coat, roots, root tips and the like.

The term “plant cell” used herein refers to a structural and physiological unit of a plant, comprising a protoplast and a cell wall. The plant cell may be in a form of an isolated single cell or a cultured cell.

The term “plant cell culture” as used herein means cultures of plant units such as, for example, protoplasts, regenerable cells, cell culture, cells, cells in plant tissues, pollen, pollen tubes, ovules, embryo sacs, zygotes and embryos at various stages of development, leaves, roots, root tips, anthers, meristematic cells, microspores, flowers, cotyledons, pistil, fruit, seeds, seed coat or any combination thereof.

The term “plant material” or “plant part” used herein refers to leaves, stems, roots, root tips, flowers or flower parts, fruits, pollen, egg cells, zygotes, seeds, seed coat, cuttings, cell or tissue cultures, or any other part or product of a plant or a combination thereof.

A “plant organ” as used herein means a distinct and visibly structured and differentiated part of a plant such as, but not limited to a root, stem, leaf, flower, flower bud, or embryo.

The term “Plant tissue” as used herein means a group of plant cells organized into a structural and functional unit. Any tissue of a plant in planta or in culture is included. This term includes, but is not limited to, plant organs, plant seeds, tissue culture, protoplasts, meristematic cells, calli and any group of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “progeny” or “progenies” refers in a non-limiting manner to offspring or descendant plants. According to certain embodiments, the term “progeny” or “progenies” refers to plants developed or grown or produced from the disclosed or deposited seeds as detailed inter alia. The grown plants preferably have the desired traits of the disclosed or deposited seeds, i.e. loss of function mutation in at least one Cowpea or Peanut SP gene.

The term “Legume” refers hereinafter to a plant in the family Fabaceae (or Leguminosae), or the fruit or seed of such a plant.

The legume family consists of plants that produce a pod with seeds inside.

The term “SELF-PRUNING” or “SP” in the context of the present invention refers to a gene which encodes a flowering repressor that modulates sympodial growth. It is herein shown that mutations in the SP orthologue cause an acceleration of sympodial cycling and shoot termination. It is further acknowledged that the SELF PRUNING (SP) gene controls the regularity of the vegetative-reproductive switch along the compound shoot of, for example, tomato, and thus conditions the ‘determinate’ (sp/sp) and ‘indeterminate’ (SP) growth habits of the plant. SP is a developmental regulator which is considered as similar to CENTRORADIALIS (CEN) from Antirrhinum and TERMINAL FLOWER 1 (TFL1) and FLOWERING LOCUS T (FT) from Arabidopsis.

The present invention discloses that SP is a member of a gene family in Legumes composed of at least 23 genes.

According to certain aspects of the present invention, the Legumes SP genes include Cowpea and Peanut SP genes.

According to one embodiment, the Cowpea (Vigna unguiculata) SP gene is selected from VuSP1-VuSP7, comprising a genomic sequence as set forth in SEQ ID NO:1, SEQ ID NO:113, SEQ ID NO:239, SEQ ID NO:340, SEQ ID NO:464, SEQ ID NO:616 and SEQ ID NO:796, respectively, and encoding a polypeptide sequence as set for in SEQ ID NO:2, SEQ ID NO:114, SEQ ID NO:240, SEQ ID NO:341, SEQ ID NO:465, SEQ ID NO:617 and SEQ ID NO:797, respectively.

The Peanut (Arachis hypogaea) SP gene is selected from AhSP1-AhSP9 comprising a genomic sequence as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and amino acid or polypeptide sequence as set forth in SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, respectively.

According to main aspects of the present invention, genome editing-targeted mutation in at least one of the aforementioned Cowpea and Peanut SP genes, which reduces the functional expression of the gene, affect the plant sympodial growth habit which plays a key role in determining plant architecture.

As used herein the term “genetic modification” refers hereinafter to genetic manipulation or modulation, which is the direct manipulation of an organism's genes using biotechnology. It also refers to a set of technologies used to change the genetic makeup of cells, including the transfer of genes within and across species, targeted mutagenesis and genome editing technologies to produce improved organisms. According to main embodiments of the present invention, modified Legume plants with improved domestication traits are generated using genome editing mechanism. This technique enables to achieve in planta modification of specific genes that relate to and/or control the flowering time and plant architecture in Legumes. The modification of the genes is aimed to result in modulated expression (preferably silencing) of the targeted genes, as compared to control plants lacking the generated modification.

The term “genome editing”, or “genome/genetic modification” or “genome engineering” or “gene editing” generally refers hereinafter to a type of genetic engineering in which DNA is inserted, deleted, modified or replaced in the genome of a living organism. Unlike previous genetic engineering techniques that randomly insert genetic material into a host genome, genome editing targets the insertions to site specific locations (e.g. specific genomic locus or loci).

It is within the scope of the present invention that the common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ) or homologous recombination (HR), resulting in targeted mutations (‘edits’). Families of engineered nucleases used by the current invention include, but are not limited to: meganucleases, zinc finger nucleases (ZFNs), transcription activator-like effector-based nucleases (TALEN), and the clustered regularly interspaced short palindromic repeats (CRISPR/Cas9) system.

Reference is now made to exemplary genome editing terms used by the current disclosure:
Cas=CRISPR-associated genes
Cas9, Csn1=a CRISPR-associated protein containing two nuclease domains, that is programmed by small RNAs to cleave DNA
crRNA=CRISPR RNA
dCAS9=nuclease-deficient Cas9
DSB=Double-Stranded Break
gRNA=guide RNA
HDR=Homology-Directed Repair
HNH=an endonuclease domain named for characteristic histidine and asparagine residues
Indel=insertion and/or deletion
NHEJ=Non-Homologous End Joining
PAM=Protospacer-Adjacent Motif
RuvC=an endonuclease domain named for an E. coli protein involved in DNA repair
sgRNA=single guide RNA
tracrRNA, trRNA=trans-activating crRNA
TALEN=Transcription-Activator Like Effector Nuclease
ZFN=Zinc-Finger Nuclease

According to specific aspects of the present invention, the CRISPR (Clustered Regularly Interspaced Short Palindromic Repeats) and CRISPR-associated (Cas) genes are used for the first time for generating genome modification in targeted genes in Legume plants. It is herein acknowledged that the functions of CRISPR (Clustered Regularly Interspaced Short Palindromic Repeats) and CRISPR-associated (Cas) genes are essential in adaptive immunity in selected bacteria and archaea, enabling the organisms to respond to and eliminate invading genetic material. These repeats were initially discovered in the 1980s in E. coli. Without wishing to be bound by theory, reference is now made to a type of CRISPR mechanism, in which invading DNA from viruses or plasmids is cut into small fragments and incorporated into a CRISPR locus comprising a series of short repeats (around 20 bps). The loci are transcribed, and transcripts are then processed to generate small RNAs (crRNA, namely CRISPR RNA), which are used to guide effector endonucleases that target invading DNA based on sequence complementarity.

According to further aspects of the invention, Cas protein, such as Cas9 (also known as Csn1) is required for gene silencing. Cas9 participates in the processing of crRNAs, and is responsible for the destruction of the target DNA. Cas9's function in both of these steps relies on the presence of two nuclease domains, a RuvC-like nuclease domain located at the amino terminus, and a HNH-like nuclease domain that resides in the mid-region of the protein. To achieve site-specific DNA recognition and cleavage, Cas9 is complexed with both a crRNA and a separate trans-activating crRNA (tracrRNA or trRNA), that is partially complementary to the crRNA. The tracrRNA is required for crRNA maturation from a primary transcript encoding multiple pre-crRNAs. This occurs in the presence of RNase III and Cas9.

Without wishing to be bound by theory, it is herein acknowledged that during the destruction of target DNA, the HNH and RuvC-like nuclease domains cut both DNA strands, generating double-stranded breaks (DSBs) at sites defined by a 20-nucleotide target sequence within an associated crRNA transcript. The HNH domain cleaves the complementary strand, while the RuvC domain cleaves the noncomplementary strand.

It is further noted that the double-stranded endonuclease activity of Cas9 also requires that a short conserved sequence, (2-5 nts) known as protospacer-associated motif (PAM), follows immediately 3″- of the crRNA complementary sequence.

According to further aspects of the invention, a two-component system may be used by the current invention, combining trRNA and crRNA into a single synthetic single guide RNA (sgRNA) for guiding targeted gene alterations.

It is further within the scope that Cas9 nuclease variants include wild-type Cas9, Cas9D10A and nuclease-deficient Cas9 (dCas9).

Reference is now made to an example of CRISPR/Cas9 mechanism of action as depicted by Xie, Kabin, and Yinong Yang. “RNA-guided genome editing in plants using a CRISPR-Cas system.” Molecular plant 6.6 (2013): 1975-1983, incorporated herein by reference. As shown in this scientific publication, the Cas9 endonuclease forms a complex with a chimeric RNA (called guide RNA or gRNA), replacing the crRNA-transcrRNA heteroduplex, and the gRNA could be programmed to target specific sites. The gRNA-Cas9 should comprise at least 15-base-pairing (gRNA seed region) without mismatch between the 5′-end of engineered gRNA and targeted genomic site, and an NGG motif (called protospacer-adjacent motif or PAM) that follows the base-pairing region in the complementary strand of the targeted DNA.

The term “meganucleases” as used herein refers hereinafter to endodeoxyribonucleases characterized by a large recognition site (double-stranded DNA sequences of 12 to 40 base pairs); as a result this site generally occurs only once in any given genome. Meganucleases are therefore considered to be the most specific naturally occurring restriction enzymes.

The term “protospacer adjacent motif” or “PAM” as used herein refers hereinafter to a 2-6 base pair DNA sequence immediately following the DNA sequence targeted by the Cas9 nuclease in the CRISPR bacterial adaptive immune system. PAM is a component of the invading virus or plasmid, but is not a component of the bacterial CRISPR locus. PAM is an essential targeting component which distinguishes bacterial self from non-self DNA, thereby preventing the CRISPR locus from being targeted and destroyed by nuclease.

The term “Next-generation sequencing” or “NGS” as used herein refers hereinafter to massively, parallel, high-throughput or deep sequencing technology platforms that perform sequencing of millions of small fragments of DNA in parallel. Bioinformatics analyses are used to piece together these fragments by mapping the individual reads to the reference genome.

The term “gene knockdown” as used herein refers hereinafter to an experimental technique by which the expression of one or more of an organism's genes is reduced. The reduction can occur through genetic modification, i.e. targeted genome editing or by treatment with a reagent such as a short DNA or RNA oligonucleotide that has a sequence complementary to either gene or an mRNA transcript. The reduced expression can be at the level of RNA and/or at the level of protein. It is within the scope of the present invention that the term gene knockdown also refers to a loss of function mutation, gene knockout or silencing mutation in which an organism's genes is made inoperative or nonfunctional.

The term “gene silencing” as used herein refers hereinafter to the regulation of gene expression in a cell to prevent the expression of a certain gene. Gene silencing can occur during either transcription or translation. In certain aspects of the invention, gene silencing is considered to have a similar meaning as gene knockdown. When genes are silenced, their expression is reduced. In contrast, when genes are knocked out, they are completely not expressed. Gene silencing may be considered a gene knockdown mechanism since the methods used to silence genes, such as RNAi, CRISPR, or siRNA, generally reduce the expression of a gene by at least 70% but do not completely eliminate it.

The term “loss of function mutation” as used herein refers to a type of mutation in which the altered gene product lacks the function of the wild-type gene. A synonyms of the term included within the scope of the present invention is null mutation.

The term “microRNAs” or “miRNAs” refers hereinafter to small non-coding RNAs that have been found in most of the eukaryotic organisms. They are involved in the regulation of gene expression at the post-transcriptional level in a sequence specific manner. MiRNAs are produced from their precursors by Dicer-dependent small RNA biogenesis pathway. MiRNAs are candidates for studying gene function using different RNA-based gene silencing techniques. For example, artificial miRNAs (amiRNAs) targeting one or several genes of interest is a potential tool in functional genomics.

The term “in planta” means in the context of the present invention within the plant or plant cells. More specifically, it means introducing CRISPR/Cas complex into plant material comprising a tissue culture of several cells, a whole plant, or into a single plant cell, without introducing a foreign gene or a mutated gene. It also used to describe conditions present in a non-laboratory environment (e.g. in vivo).

The term ‘sympodial growth’ as used herein refers to a type of bifurcating branching pattern where one branch develops more strongly than the other, resulting in the stronger branches forming the primary shoot and the weaker branches appearing laterally. A sympodium, also referred to as a sympode or pseudaxis, is the primary shoot, comprising the stronger branches, formed during sympodial growth. In some aspects of the present invention, sympodial growth occurs when the apical meristem is terminated and growth is continued by one or more lateral meristems, which repeat the process. The apical meristem may be consumed to make an inflorescence or other determinate structure, or it may be aborted.

It is further within the scope of the current invention that the shoot section between two successive inflorescences is called the ‘sympodium’, and the number of leaf nodes per sympodium is referred to as the ‘sympodial index’ (spi). The first termination event activates the ‘sympodial cycle’. In sympodial plants, the apparent main shoot consists of a reiterated array of ‘sympodial units’. A mutant sp gene accelerates the termination of sympodial units but does not change the sympodial habit. The result is a progressive reduction in the number of vegetative nodes between inflorescences in a pattern that depends on light intensity and genetic background.

The term “earliness” refers hereinafter to early flowering and/or rapid transition from the vegetative to reproductive stages, or reduced ‘time to initiation of flowering’ and more generally to earlier completion of the life-cycle.

Plants having an “early flowering time” as used herein are plants which start to flower earlier than control plants. Hence this term refers to plants that show an earlier start of flowering. Flowering time of plants can be assessed by counting the number of days (“time to flower”) between sowing and the emergence of a first inflorescence. The “flowering time” of a plant can be determined using any method known in the art.

The term ‘reduced flowering time’ as used herein refers to time to production of first inflorescence. Such a trait can be evaluated or measured, for example, with reference to the number of leaves produced prior to appearance of the first inflorescence.

The term ‘harvest index’ can be herein defined as the total yield per plant weight.

The term ‘day length’ or ‘day length sensitivity’ as used in the context of the present invention generally refers to photoperiodism, which is the physiological reaction of organisms to the length of day or night. Photoperiodism can also be defined as the developmental responses of plants to the relative lengths of light and dark periods. Plants are classified under three groups according to the photoperiods: short-day plants, long-day plants, and day-neutral plants. Photoperiodism affects flowering by inducing the shoot to produce floral buds instead of leaves and lateral buds. It is within the scope of the present invention that Legumes are included within the short-day facultative plants. The Legume plants of the present invention are genetically modified so as to exhibit loss of day-length sensitivity, which is a highly desirable agronomical trait enabling enhanced yield of the cultivated crop.

The term ‘determinate’ or ‘determinate growth’ as used herein refers to plant growth in which the main stem ends in an inflorescence or other reproductive structure (e.g. a bud) and stops continuing to elongate indefinitely with only branches from the main stem having further and similarly restricted growth. It also refers to growth characterized by sequential flowering from the central or uppermost bud to the lateral or basal buds. It further means naturally self-limited growth, resulting in a plant of a definite maximum size.

The term ‘indeterminate’ or ‘indeterminate growth’ as used herein refers to plant growth in which the main stem continues to elongate indefinitely without being limited by a terminal inflorescence or other reproductive structure. It also refers to growth characterized by sequential flowering from the lateral or basal buds to the central or uppermost buds.

It is within the scope of the present invention that ‘yield related traits’ comprise one or more of early flowering time, yield, biomass, seed yield, early vigour, greenness index, increased growth rate and improved agronomic traits (such as improved plant architecture, i.e. determinate growth habit and enhanced nutritional value).

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The terms “yield” of a plant and “plant yield” are used interchangeably herein and are meant to refer to vegetative biomass such as root and/or shoot biomass, to reproductive organs, and/or to propagules such as seeds of that plant.

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield, growth or any other agronomic trait such as domestication trait or plant architecture, in comparison to control plants as defined herein.

Increased seed yield may be defined as one or more of the following: (a) an increase in seed biomass (total seed weight) which may be on an individual seed basis and/or per plant and/or per square meter; (b) increased number of flowers per plant; (c) increased number of seeds; and (d) increased harvest index, which is expressed as a ratio of the yield of harvestable parts, such as seeds, divided by the biomass of aboveground plant parts.

An increase in seed yield may also be manifested as an increase in seed size and/or seed volume.

The term “biomass” as used herein is intended to refer to the total weight of a plant. Within the definition of biomass, a distinction may be made between the biomass of one or more parts of a plant, which may include: aboveground (harvestable) parts such as but not limited to shoot biomass, seed biomass, leaf biomass, etc. and/or (harvestable) parts below ground, such as but not limited to root biomass, etc., and/or vegetative biomass such as root biomass, shoot biomass, etc., and/or reproductive organs, and/or propagules such as seed.

Control plant(s) within the scope of the present invention include corresponding wild type plants or corresponding naturally occurring plants or corresponding plants lacking the edited or mutated gene of interest or the specific generated mutation. The choice of suitable control plants is a routine part of an experimental setup and may include corresponding wild type plants or corresponding plants without the gene of interest. The control plant is typically of the same plant species or the same genetic background or even of the same variety as the plant to be assessed. The control plant of the plant to be assessed may also be plant individuals missing the transgene or modified/edited gene. A “control plant” or a “wild type” plant as used herein refers not only to whole plants, but also to plant parts, including seeds and seed parts.

The term “orthologue” as used herein refers hereinafter to one of two or more homologous gene sequences found in different species.

The term “functional variant” or “functional variant of a nucleic acid or amino acid sequence” as used herein refers to a sequence or part of a sequence which retains the biological function of the full non-variant allele (e.g. Cowpea or Peanut SP genes) and hence has the activity of SP expressed gene or protein. A functional variant also comprises a variant of the gene of interest encoding a polypeptide which has sequence alterations that do not affect function of the resulting protein, for example, in non-conserved residues. Also encompassed is a variant that is substantially identical, i.e. has only some sequence variations, for example, in non-conserved residues, to the wild type nucleic acid or amino acid sequences of the alleles as shown herein, and is biologically active.

The term ‘resistant’ or ‘less sensitive’ as used herein refers to plant growth in which the plant either expresses no symptoms or less symptoms of a disease conferred by an associated pathogen in comparison to a plant that is sensitive to such pathogen.

The term “variety” or “cultivar” used herein means a group of similar plants that by structural features and performance can be identified from other varieties within the same species.

The term “allele” used herein means any of one or more alternative or variant forms of a gene or a genetic unit at a particular locus, all of which alleles relate to one trait or characteristic at a specific locus. In a diploid cell of an organism, alleles of a given gene are located at a specific location, or locus (loci plural) on a chromosome. Alternative or variant forms of alleles may be the result of single nucleotide polymorphisms, insertions, inversions, translocations or deletions, or the consequence of gene regulation caused by, for example, by chemical or structural modification, transcription regulation or post-translational modification/regulation. An allele associated with a qualitative trait may comprise alternative or variant forms of various genetic units including those that are identical or associated with a single gene or multiple genes or their products or even a gene disrupting or controlled by a genetic factor contributing to the phenotype represented by the locus. According to further embodiments, the term “allele” designates any of one or more alternative forms of a gene at a particular locus. Heterozygous alleles are two different alleles at the same locus. Homozygous alleles are two identical alleles at a particular locus. A wild type allele is a naturally occurring allele. In the context of the current invention, the term allele refers to the 16 identified SP legumes genes, having the genomic nucleotide sequence as set forth in SEQ ID NO:1, SEQ ID NO:113, SEQ ID NO:239, SEQ ID NO:340, SEQ ID NO:464, SEQ ID NO:616 and SEQ ID NO:796 for Cowpea SP, and SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut SP.

As used herein, the term “locus” (loci plural) means a specific place(s) or region(s) or a site(s) on a chromosome where for example a gene or genetic marker element or factor is found. In specific embodiments, such a genetic element is contributing to a trait.

As used herein, the term “homozygous” refers to a genetic condition or configuration existing when two identical or like alleles reside at a specific locus, but are positioned individually on corresponding pairs of homologous chromosomes in the cell of a diploid organism.

In specific embodiments, the Legume plants of the present invention comprise homozygous configuration of at least one of the mutated Cowpea or Peanut SP genes.

Conversely, as used herein, the term “heterozygous” means a genetic condition or configuration existing when two different or unlike alleles reside at a specific locus, but are positioned individually on corresponding pairs of homologous chromosomes in the cell of a diploid organism.

As used herein, the phrase “genetic marker” or “molecular marker” or “biomarker” refers to a feature in an individual's genome e.g., a nucleotide or a polynucleotide sequence that is associated with one or more loci or trait of interest In some embodiments, a genetic marker is polymorphic in a population of interest, or the locus occupied by the polymorphism, depending on context. Genetic markers or molecular markers include, for example, single nucleotide polymorphisms (SNPs), indels (i.e. insertions deletions), simple sequence repeats (SSRs), restriction fragment length polymorphisms (RFLPs), random amplified polymorphic DNAs (RAFDs), cleaved amplified polymorphic sequence (CAPS) markers, Diversity Arrays Technology (DArT) markers, and amplified fragment length polymorphisms (AFLPs) or combinations thereof, among many other examples such as the DNA sequence per se. Genetic markers can, for example, be used to locate genetic loci containing alleles on a chromosome that contribute to variability of phenotypic traits. The phrase “genetic marker” or “molecular marker” or “biomarker” can also refer to a polynucleotide sequence complementary or corresponding to a genomic sequence, such as a sequence of a nucleic acid used as a probe or primer.

As used herein, the term “germplasm” refers to the totality of the genotypes of a population or other group of individuals (e.g., a species). The term “germplasm” can also refer to plant material; e.g., a group of plants that act as a repository for various alleles. Such germplasm genotypes or populations include plant materials of proven genetic superiority; e.g., for a given environment or geographical area, and plant materials of unknown or unproven genetic value; that are not part of an established breeding population and that do not have a known relationship to a member of the established breeding population.

The terms “hybrid”, “hybrid plant” and “hybrid progeny” used herein refers to an individual produced from genetically different parents (e.g., a genetically heterozygous or mostly heterozygous individual).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. The term further refers hereinafter to the amount of characters which match exactly between two different sequences. Hereby, gaps are not counted and the measurement is relational to the shorter of the two sequences.

It is further within the scope that the terms “similarity” and “identity” additionally refer to local homology, identifying domains that are homologous or similar (in nucleotide and/or amino acid sequence). It is acknowledged that bioinformatics tools such as BLAST, SSEARCH, FASTA, and HMMER calculate local sequence alignments which identify the most similar region between two sequences. For domains that are found in different sequence contexts in different proteins, the alignment should be limited to the homologous domain, since the domain homology is providing the sequence similarity captured in the score. According to some aspects the term similarity or identity further includes a sequence motif, which is a nucleotide or amino-acid sequence pattern that is widespread and has, or is conjectured to have, a biological significance. Proteins may have a sequence motif and/or a structural motif, a motif formed by the three-dimensional arrangement of amino acids which may not be adjacent.

As used herein, the terms “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, in their gene expressed form, and/or may include cDNAs in combination with regulatory sequences. Thus, according to the various aspects of the invention, genomic DNA, cDNA or coding DNA may be used. In one embodiment, the nucleic acid is cDNA or coding DNA.

The terms “peptide”, “polypeptide” and “protein” are used interchangeably herein and refer to amino acids in a polymeric form of any length, linked together by peptide bonds.

According to other aspects of the invention, a “modified” or a “mutant” plant is a plant that has been altered compared to the naturally occurring wild type (WT) or control plant. Specifically, the endogenous nucleic acid sequences of each of the SP homologs in Legumes (nucleic acid sequences comprising at least 75% sequence identity to SEQ ID NO:1, SEQ ID NO:113, SEQ ID NO:239, SEQ ID NO:340, SEQ ID NO:464, SEQ ID NO:616 and SEQ ID NO:796 for Cowpea SP genes, and at least 75% sequence identity to SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut SP genes) have been altered compared to wild type sequences using mutagenesis and/or genome editing methods as described herein. This causes inactivation of the endogenous SP gene and thus disables SP function and/or expression. Such plants have an altered phenotype and show improved domestication traits such as determinant plant architecture, synchronous and/or early flowering and loss of day length sensitivity compared to corresponding wild type plants or control plants lacking the SP modification. Therefore, the improved domestication phenotype is conferred by the presence of at least one mutated endogenous Cowpea or Peanut SP gene in the Legumes plant genome which has been specifically targeted using genome editing technique.

According to further aspects of the present invention, the at least one improved domestication trait is not conferred by the presence of transgenes expressed in Legumes.

It is further within the scope of the current invention that sp mutations that down-regulate or disrupt functional expression of the wild-type SP gene sequence, may be recessive, such that they are complemented by expression of a wild-type sequence.

It is further noted that according to certain aspects of the present invention, a wild type Legume plant is a plant that does not have any mutant sp allele.

Main aspects of the invention involve targeted mutagenesis methods, specifically genome editing, and exclude embodiments that are solely based on generating plants by traditional breeding methods. In a further embodiment of the current invention, as explained herein, the improved domestication of at least one trait is not due to the presence of a transgene.

The inventors have generated mutant Legume lines with mutations inactivating at least one Cowpea or Peanut SP gene homoeoallele which confer heritable improved domestication trait(s). In this way no functional at least one Cowpea or Peanut SP protein is made. Thus, the invention relates to these mutant Legume lines and related methods.

It is further within the scope of the present invention that breeding Legume cultivars with mutated sp allele enables the mechanical harvest of the plant. According to a further aspect of the present invention, loss of SP function results in compact Legume plants with reduced height, reduced number of sympodial units and determinate growth when compared with corresponding WT Legumes.

According to a main aspect of the present invention, modifying Legumes shoot architecture by selection for mutations in florigen flowering pathway genes allowed major improvements in plant architecture and yield. In particular, a mutation in an antiflorigen SELFPRUNING (SP) gene (sp classic) provided compact ‘determinate’ growth that translated to a burst of flowers, thereby enabling largescale field production.

The work inter alia described has important implications. The results have shown that CRISPR/Cas9 can be used to create heritable mutations in florigen pathway family members that result in desirable phenotypic effects.

To edit multiple domestication genes simultaneously and stack the resulting allelic variants, one option is that several gRNAs can be assembled to edit several genes into one construct, by using the Csy4 multi-gRNA system. The construct is then transformed via an appropriate vector into several Legume accessions.

It is further within the scope of the current invention that Legume SP genes having genomic nucleotide sequence as set forth in SEQ ID NO:1, SEQ ID NO:113, SEQ ID NO:239, SEQ ID NO:340, SEQ ID NO:464, SEQ ID NO:616 and SEQ ID NO:796 for cowpea, SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut, are silenced by genome editing. Several mutated alleles have been identified. Notably, the plants with mutated sp alleles were more compact than the wild type plants lacking the mutated allele.

The loss of function mutation may be a deletion or insertion (“indels”) with reference the wild type Cowpea or Peanut SP gene allele sequence. The deletion may comprise 1-20 or more nucleotides, for example 1, 2, 3, 4, 5, 6, 7, 8, 9, 10, 1, 12, 13, 14, 15, 16, 17, 18 or 20 nucleotides or more in one or more strand. The insertion may comprise 1-20 or more nucleotides, for example 1, 2, 3, 4, 5, 6, 7, 8, 9, 10, 1, 12, 13, 14, 15, 16, 17, 18 or 20 or more nucleotides in one or more strand.

The plant of the invention includes plants wherein the plant is heterozygous for the each of the mutations. In a preferred embodiment however, the plant is homozygous for the mutations. Progeny that is also homozygous can be generated from these plants according to methods known in the art.

It is further within the scope that variants of a particular Cowpea or Peanut SP gene nucleotide or amino acid encoded sequence according to the various aspects of the invention will have at least about 50%-99%, for example at least 75%, for example at least 85%, 86%, 87%, 88%, 89%, 90%, 92%, 94%, 95%, 96%, 97%, 98% or 99% or more sequence identity to that particular non-variant Cowpea or Peanut SP gene nucleotide sequence as shown in SEQ ID NO:1, SEQ ID NO:113, SEQ ID NO:239, SEQ ID NO:340, SEQ ID NO:464, SEQ ID NO:616 and SEQ ID NO:796 for cowpea, or SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for peanut.

Also, the various aspects of the invention encompass not only a Cowpea and Peanut SP genes or any other aforementioned nucleic acid sequence or amino acid sequence, but also fragments thereof. By “fragment” it is intended to mean a portion of the nucleotide sequence or a portion of the amino acid sequence and hence of the protein encoded thereby. Fragments of a nucleotide sequence may encode protein fragments that retain the biological activity of the native protein, in this case improved domestication trait.

According to a further embodiment of the invention, the herein newly identified Legume SP (Cowpea and Peanut SP genes) have been targeted using the double sgRNA strategy.

According to further embodiments of the present invention, DNA introduction into the plant cells can be done by Agrobacterium infiltration, virus based plasmids for delivery of the genome editing molecules and mechanical insertion of DNA (PEG mediated DNA transformation, biolistics, etc.).

In addition, it is within the scope of the present invention that the Cas9 protein is directly inserted together with a gRNA (ribonucleoprotein-RNP's) in order to bypass the need for in vivo transcription and translation of the Cas9+gRNA plasmid in planta to achieve gene editing.

It is also possible to create a genome edited plant and use it as a rootstock. Then, the Cas protein and gRNA can be transported via the vasculature system to the top of the plant and create the genome editing event in the scion.

It is within the scope of the present invention that the usage of CRISPR/Cas system for the generation of Legume plants with at least one improved domestication trait, allows the modification of predetermined specific DNA sequences without introducing foreign DNA into the genome by GMO techniques. According to one embodiment of the present invention, this is achieved by combining the Cas nuclease (e.g. Cas9, Cpf1 and the like) with a predefined guide RNA molecule (gRNA). The gRNA is complementary to a specific DNA sequence targeted for editing in the plant genome and which guides the Cas nuclease to a specific nucleotide sequence. The predefined gene specific gRNA's are cloned into the same plasmid as the Cas gene and this plasmid is inserted into plant cells. Insertion of the aforementioned plasmid DNA can be done, but not limited to, using different delivery systems, biological and/or mechanical, e.g. Agrobacterium infiltration, virus based plasmids for delivery of the genome editing molecules and mechanical insertion of DNA (PEG mediated DNA transformation, biolistics, etc.).

It is further within the scope of the present invention that upon reaching the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which eventually create a mutation at the cleavage site. For example, it is acknowledged that a deletion form of the mutation consists of at least 1 base pair deletion. As a result of this base pair deletion the gene coding sequence is disrupted and the translation of the encoded protein is compromised either by a premature stop codon or disruption of a functional or structural property of the protein. Thus DNA is cut by the Cas9 protein and re-assembled by the cell's DNA repair mechanism.

It is further within the scope that improved domestication traits in Legume plants is herein produced by generating gRNA with homology to a specific site of predetermined genes in the Legumes genome i.e. SP gene, sub cloning this gRNA into a plasmid containing the Cas9 gene, and insertion of the plasmid into the Legume plant cells. In this way site specific mutations in the SP and aforementioned genes are generated thus effectively creating non-active molecules, resulting in determinant growth habit of the genome edited plant.

According to one embodiment, the present invention provides a modified Legume plant exhibiting at least one improved domestication trait as compared to a corresponding control Legume plant, wherein said modified plant comprises a mutated SELF PRUNING (SP) gene selected from Cowpea (Vigna unguiculata) SP gene or Peanut (Arachis hypogaea) SP gene.

According to a further embodiment, the present invention discloses the modified Legume plant as defined in any of the above, wherein said Cowpea (Vigna unguiculata) SP gene is selected from VuSP1-VuSP7 comprising a nucleic acid sequence with at least 75% sequence identity to a sequence selected from SEQ ID NO:1, SEQ ID NO:113, SEQ ID NO:239, SEQ ID NO:340, SEQ ID NO:464, SEQ ID NO:616 and SEQ ID NO:796, respectively, or a functional variant thereof and any combination thereof.

According to a further embodiment, the present invention discloses the modified Legume plant as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or a functional variant thereof and any combination thereof.

According to a further embodiment, the present invention discloses the modified Legume plant as defined in any of the above, wherein said mutation is introduced using mutagenesis, small interfering RNA (siRNA), microRNA (miRNA), artificial miRNA (amiRNA), DNA introgression, endonucleases or any combination thereof.

According to a further embodiment, the present invention discloses the modified Legume plant as defined in any of the above, wherein said mutation is introduced using targeted genome modification.

According to a further embodiment, the present invention discloses the modified Legume plant as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

According to a further embodiment, the present invention discloses the modified Legume plant as defined in any of the above, wherein said Cas gene is selected from the group consisting of Cas9, Cas12, Cas13, Cas14, CasX, CasY, Csn1, Cpf1 and any combination thereof.

According to a further embodiment, the present invention discloses the modified Legume plant as defined in any of the above, wherein the mutated SP gene is a CRISPR/Cas9-induced heritable mutated allele.

According to a further embodiment, the present invention discloses the modified Legume plant as defined in any of the above, wherein said mutation is a missense mutation, nonsense mutation, insertion, deletion, indel, substitution or duplication.

According to a further embodiment, the present invention discloses the modified Legume plant as defined in any of the above, wherein the insertion or the deletion produces a gene comprising a frameshift.

According to a further embodiment, the present invention discloses the modified Legume plant as defined in any of the above, wherein said plant is homozygous for said at least one mutated SP gene.

According to a further embodiment, the present invention discloses the modified Legume plant as defined in any of the above, wherein said mutation is in the coding region of said gene, a mutation in the regulatory region of said gene, or an epigenetic factor.

According to a further embodiment, the present invention discloses the modified Legume plant as defined in any of the above, wherein said mutation is a silencing mutation, a knockdown mutation, a knockout mutation, a loss of function mutation or any combination thereof.

According to a further embodiment, the present invention discloses the modified Legume plant as defined in any of the above, wherein said mutation is generated in planta.

According to a further embodiment, the present invention discloses the modified Legume plant as defined in any of the above, wherein said mutation is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for said at least one Peanut SP gene, and any combination thereof.

According to a further embodiment, the present invention discloses the modified Legume plant as defined in any of the above, wherein said mutation in said at least one Cowpea and at least one Peanut SP gene is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, and any combination thereof.

According to a further embodiment, the present invention discloses the modified Legume plant as defined in any of the above, wherein said gRNA sequence comprises a 3′ NGG Protospacer Adjacent Motif (PAM).

According to a further embodiment, the present invention discloses the modified Legume plant as defined in any of the above, wherein said construct is introduced into the plant cells via Agrobacterium infiltration, virus-based plasmids for delivery of genome editing molecules, or mechanical insertion such as polyethylene glycol (PEG) mediated DNA transformation, electroporation or gene gun biolistics.

According to a further embodiment, the present invention discloses the modified Legume plant as defined in any of the above, wherein said mutation confers reduced expression of said at least one SP gene.

According to a further embodiment, the present invention discloses the modified Legume plant as defined in any of the above, wherein said modified plant has decreased expression levels of said SP gene.

According to a further embodiment, the present invention discloses the modified Legume plant as defined in any of the above, wherein the sequence of said expressed SP gene is selected from the group consisting of: at least 75% identity to any one of Cowpea polypeptide SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the modified Legume plant as defined in any of the above, wherein said SP gene encodes a polypeptide sequence selected from the group consisting of: at least 75% identity to any one of Cowpea SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the modified Legume plant as defined in any of the above, wherein said modified plant is semi-determinant.

According to a further embodiment, the present invention discloses the modified Legume plant as defined in any of the above, wherein said modified plant has determinant growth habit.

According to a further embodiment, the present invention discloses the modified Legume plant as defined in any of the above, wherein said modified plant flowers earlier than a corresponding control Legume plant lacking said mutated SP gene.

According to a further embodiment, the present invention discloses the modified Legume plant as defined in any of the above, wherein said modified plant exhibits improved earliness as compared to a corresponding control Legume plant lacking said mutated SP gene.

According to a further embodiment, the present invention discloses the modified Legume plant as defined in any of the above, wherein said modified plant exhibits suppressed and/or similar sympodial shoot termination as compared to a corresponding control Legume plant lacking said mutated SP gene.

According to a further embodiment, the present invention discloses the modified Legume plant as defined in any of the above, wherein said domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

According to a further embodiment, the present invention discloses a modified Legume plant, plant part, plant tissue or plant cell as defined in any of the above, wherein said plant does not comprise a transgene.

According to a further embodiment, the present invention discloses a plant part, plant cell, plant pod or plant seed of a modified Legume plant as defined in any of the above.

According to a further embodiment, the present invention discloses a tissue culture of regenerable cells, protoplasts or callus obtained from the modified Legume plant as defined in any of the above.

According to a further embodiment, the present invention discloses a method for producing a modified Legume plant exhibiting at least one improved domestication trait compared with a corresponding control Legume, said method comprises steps of genetically modifying at least one Legume SELF PRUNING (SP) gene selected from Cowpea (Vigna unguiculata) SP gene or Peanut (Arachis hypogaea) SP gene, the resultant mutated SP gene has reduced expression level.

According to a further embodiment, the present invention discloses the method as defined in any of the above, wherein said method comprises steps of genetically modifying the at least one Legume SP gene using targeted genome editing introducing a loss of function mutation in the at least one Cowpea (Vigna unguiculata) SP gene or Peanut (Arachis hypogaea) SP gene.

According to a further embodiment, the present invention discloses the method as defined in any of the above, wherein said Cowpea (Vigna unguiculata) SP gene is selected from VuSP1-VuSP7 comprising a nucleic acid sequence with at least 75% sequence identity to a sequence selected from SEQ ID NO:1, SEQ ID NO:113, SEQ ID NO:239, SEQ ID NO:340, SEQ ID NO:464, SEQ ID NO:616 and SEQ ID NO:796, respectively, or a functional variant thereof and any combination thereof.

According to a further embodiment, the present invention discloses the method as defined in any of the above, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

According to a further embodiment, the present invention discloses the method as defined in any of the above, wherein said method comprises steps of: (a) identifying at least one Legume SP gene in a predetermined Legume plant; (b) synthetizing at least one guide RNA (gRNA) comprising a nucleotide sequence complementary to said at least one identified Legume SP gene; (c) transforming the predetermined Legume plant cells with a construct comprising (i) Cas nucleotide sequence operably linked to said at least one gRNA, or (ii) a ribonucleoprotein (RNP) complex comprising Cas protein and said at least one gRNA; (d) screening the genome of said transformed predetermined Legume plant cells for induced targeted loss of function mutation in said at least one Legume SP gene; (e) regenerating Legume plants carrying said loss of function mutation in at least one of said Legume SP gene; and (f) screening said regenerated plants for a Legume plant with improved domestication trait.

According to a further embodiment, the present invention discloses the method as defined in any of the above, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said Legume SP gene.

According to a further embodiment, the present invention discloses the method as defined in any of the above, wherein said SP gene is selected from the group consisting of Cowpea SP genes comprising a sequence having at least 75% identity to a sequence selected from SEQ ID NO:1, SEQ ID NO:113, SEQ ID NO:239, SEQ ID NO:340, SEQ ID NO:464, SEQ ID NO:616 and SEQ ID NO:796, or Peanut SP gene comprising a sequence having at least 75% identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

According to a further embodiment, the present invention discloses the method as defined in any of the above, wherein said mutation is introduced using mutagenesis, small interfering RNA (siRNA), microRNA (miRNA), artificial miRNA (amiRNA), DNA introgression, endonucleases or any combination thereof.

According to a further embodiment, the present invention discloses the method as defined in any of the above, wherein said mutation is introduced using targeted genome modification.

According to a further embodiment, the present invention discloses the method as defined in any of the above, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

According to a further embodiment, the present invention discloses the method as defined in any of the above, wherein said Cas gene is selected from the group consisting of Cas9, Cas12, Cas13, Cas14, CasX, CasY, Csn1, Cpf1 and any combination thereof.

According to a further embodiment, the present invention discloses the method as defined in any of the above, wherein the mutated SP gene is a CRISPR/Cas9-induced heritable mutated allele.

According to a further embodiment, the present invention discloses the method as defined in any of the above, wherein said mutation is a missense mutation, nonsense mutation, insertion, deletion, indel, substitution or duplication.

According to a further embodiment, the present invention discloses the method as defined in any of the above, wherein the insertion or the deletion produces a gene comprising a frameshift.

According to a further embodiment, the present invention discloses the method as defined in any of the above, wherein said plant is homozygous for said at least one Legume SP gene.

According to a further embodiment, the present invention discloses the method as defined in any of the above, wherein said mutation is in the coding region of said gene, a mutation in the regulatory region of said gene, or an epigenetic factor.

According to a further embodiment, the present invention discloses the method as defined in any of the above, wherein said mutation is a silencing mutation, a knockdown mutation, a knockout mutation, a loss of function mutation or any combination thereof.

According to a further embodiment, the present invention discloses the method as defined in any of the above, wherein said mutation is generated in planta.

According to a further embodiment, the present invention discloses the method as defined in any of the above, wherein said mutation is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof.

According to a further embodiment, the present invention discloses the method as defined in any of the above, wherein said mutation in said Cowpea and Peanut SP genes is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively.

According to a further embodiment, the present invention discloses the method as defined in any of the above, wherein said gRNA sequence comprises a 3′ NGG Protospacer Adjacent Motif (PAM).

According to a further embodiment, the present invention discloses the method as defined in any of the above, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.

According to a further embodiment, the present invention discloses the method as defined in any of the above, wherein said modified plant has decreased expression levels of at least one of said Legume SELF PRUNING (SP) gene.

According to a further embodiment, the present invention discloses the method as defined in any of the above, wherein said mutation confers reduced expression of said at least one SP gene.

According to a further embodiment, the present invention discloses the method as defined in any of the above, wherein the sequence of said expressed Legume SELF PRUNING (SP) gene is selected from the group consisting of: at least 75% identity to any one of Cowpea polypeptide SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the method as defined in any of the above, wherein the Legume SELF PRUNING (SP) gene encodes a polypeptide sequence selected from the group consisting of: at least 75% identity to any one of Cowpea polypeptide SEQ ID NO:2, SEQ ID NO:114, SEQ ID NO:240, SEQ ID NO:341, SEQ ID NO:465, SEQ ID NO:617 and SEQ ID NO:797, and at least 75% identity to any one of Peanut polypeptide SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, or a functional variant thereof.

According to a further embodiment, the present invention discloses the method as defined in any of the above, wherein said modified plant is semi-determinant.

According to a further embodiment, the present invention discloses the method as defined in any of the above, wherein said modified plant has determinant growth habit.

According to a further embodiment, the present invention discloses the method as defined in any of the above, wherein said modified plant flowers earlier than a corresponding control Legume plant lacking said mutated SP gene.

According to a further embodiment, the present invention discloses the method as defined in any of the above, wherein said modified plant exhibits improved earliness as compared to a corresponding control Legume plant lacking said mutated SP gene.

According to a further embodiment, the present invention discloses the method as defined in any of the above, wherein said modified plant exhibits suppressed sympodial shoot termination as compared to a corresponding control Legume plant lacking said mutated SP gene.

According to a further embodiment, the present invention discloses the method as defined in any of the above, wherein said modified plant exhibits similar sympodial shoot termination as compared to a corresponding control Legume plant lacking said mutated SP gene.

According to a further embodiment, the present invention discloses the method as defined in any of the above, wherein said modified plant exhibits suppressed or reduced day-length sensitivity as compared to a corresponding control Legume plant lacking said mutated SP gene.

According to a further embodiment, the present invention discloses a modified Legume plant, plant part or plant cell produced by the method as defined in any of the above, wherein said modified plant does not comprise a transgene.

According to a further embodiment, the present invention discloses a plant part, plant cell, plant pod or plant seed of a modified Legume plant produced by the method as defined in any of the above.

According to a further embodiment, the present invention discloses a tissue culture of regenerable cells, protoplasts or callus obtained from the modified Legume plant produced by the method as defined in any of the above.

According to a further embodiment, the present invention discloses the method as defined in any of the above, wherein said at least one domestication trait is selected from the group consisting of reduced flowering time, earliness, synchronous flowering, reduced day-length sensitivity, determinant or semi-determinant architecture, early termination of sympodial cycling, earlier axillary shoot flowering, compact growth habit, reduced height, reduced number of sympodial units, adaptation to mechanical harvest, higher harvest index and any combination thereof.

According to a further embodiment, the present invention discloses an isolated polynucleotide sequence comprising at least 75% identity to a Legume SELF PRUNING (SP) sequence selected from the group consisting of SEQ ID NO:1, SEQ ID NO:113, SEQ ID NO:239, SEQ ID NO:340, SEQ ID NO:464, SEQ ID NO:616, SEQ ID NO:796, SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928.

According to a further embodiment, the present invention discloses an isolated polypeptide sequence comprising at least 75% identity to a Legume SELF PRUNING (SP) sequence selected from the group consisting of SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929.

According to a further embodiment, the present invention discloses an isolated nucleotide sequence comprising at least 75% sequence identity to a Legume SELF PRUNING (SP)-targeted gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795, SEQ ID NO:798-1024, SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051.

According to a further embodiment, the present invention discloses harvestable parts of a modified Legume plant as defined in any of the above, wherein said harvestable parts are preferably shoot biomass and/or seeds.

According to a further embodiment, the present invention discloses products derived from a modified comprising at least 75% sequence identity to plant as defined in any of the above, and/or from harvestable parts of a modified Legume plant as defined in any of the above.

According to a further embodiment, the present invention discloses use of a nucleic acid encoding a polypeptide comprising at least 75% sequence identity to the sequence as defined in SEQ ID NO:2, SEQ ID NO:114, SEQ ID NO:240, SEQ ID NO:341, SEQ ID NO:465, SEQ ID NO:617, SEQ ID NO:797, SEQ ID NO:1026, SEQ ID NO:1099, SEQ ID NO:1181, SEQ ID NO:1291, SEQ ID NO:1410, SEQ ID NO:1530, SEQ ID NO:1641, SEQ ID NO:1750 and SEQ ID NO:1929, in enhancing yield and/or domestication, in Legume plants, relative to control plants.

In order to understand the invention and to see how it may be implemented in practice, a plurality of preferred embodiments will now be described, by way of non-limiting example only, with reference to the following examples.

### Example 1

Production of Legume Plants with Improved Domestication Traits by Targeted Genome Editing

Production of Legume lines with mutated sp gene may be achieved by at least one of the following breeding/cultivation schemes:

### Scheme 1:

line stabilization by self pollination
Generation of F6 parental lines
Genome editing of parental lines
Crossing edited parental lines to generate an F1 hybrid plant

### Scheme 2:

Identifying genes/alleles of interest
Designing gRNA
Transformation of plants with Cas9+gRNA constructs
Screening and identifying editing events
Genome editing of parental lines

It is noted that line stabilization may be performed by the following:
Induction of male flowering on plants
Self pollination

According to some embodiments of the present invention, line stabilization requires about 6 self-crossing (6 generations) and done through a single seed descent (SSD) approach.

F1 hybrid seed production: Novel hybrids are produced by crosses between different Legume strains.

According to a further aspect of the current invention, shortening line stabilization is performed by Doubled Haploids (DH). More specifically, the CRISPR-Cas9 system is transformed into microspores to achieve DH homozygous parental lines. A doubled haploid (DH) is a genotype formed when haploid cells undergo chromosome doubling. Artificial production of doubled haploids is important in plant breeding. It is herein acknowledged that conventional inbreeding procedures take about six generations to achieve approximately complete homozygosity, whereas doubled haploidy achieves it in one generation.

It is within the scope of the current invention that genetic markers specific for Legumes are developed and provided by the current invention:
Genotyping markers—germplasm used in the current invention is genotyped using molecular markers, in order to allow a more efficient breeding process and identification of the SP editing event.

It is further within the scope of the current invention that allele and genetic variation is analyzed for the Legume strains used.

Reference is now made to optional stages that have been used for the production of mutated SP Legume plants by genome editing:

### Stage 1: Identifying Cowpea and Peanut SP Genes.

16 SP orthologues have herein been identified in Cowpea and Peanut. These homologous genes have been sequenced and mapped. Cowpea and Peanut SP genes as set forth in SEQ ID NO:1, SEQ ID NO:113, SEQ ID NO:239, SEQ ID NO:340, SEQ ID NO:464, SEQ ID NO:616 and SEQ ID NO:796 for cowpea, and SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928 for Peanut.

Stage 2: Designing and Synthesizing gRNA Molecules Corresponding to the Sequence Targeted for Editing, i.e. Sequences of Each of the Cowpea and Peanut SP Genes.

It is noted that the editing event is preferably targeted to a unique restriction site sequence to allow easier screening for plants carrying an editing event within their genome. According to some aspects of the invention, the nucleotide sequence of the gRNAs should be completely compatible with the genomic sequence of the target gene. Therefore, for example, suitable gRNA molecules should be constructed for different SP homologues of different Legume strains.

Reference is now made to sequences of gRNA molecules targeted for silencing Cowpea and Peanut SP genes. Specifically, gRNA sequences SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024, are targeted for Cowpea SP genes VuSP1-VuSP7, respectively, comprising sequence as set forth in SEQ ID NO:1, SEQ ID NO:113, SEQ ID NO:239, SEQ ID NO:340, SEQ ID NO:464, SEQ ID NO:616 and SEQ ID NO:796, respectively. gRNA sequences SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 are targeted for Peanut SP genes AhSP1-AhSP9, respectively, comprising sequence as set forth in SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively. The term ‘PAM’ refers hereinafter to Protospacer Adjacent Motif, which is a 2-6 base pair DNA sequence immediately following the DNA sequence targeted by the Cas9 nuclease in the CRISPR bacterial adaptive immune system.

Reference is made to Tables 1 and 2 presenting a summary of the sequences within the scope of the current invention.

TABLE 1
Summary of Cowpea (Vigna unguiculata) sequences
within the scope of the present invention

| | VuSP1 | VuSP2 | VuSP3 | VuSP4 | VuSP5 | VuSP6 | VuSP7 |
|---|---|---|---|---|---|---|---|
| Sequence | SEQ ID | SEQ ID | SEQ ID | SEQ ID | SEQ ID | SEQ ID | SEQ ID |
| type | NO | NO | NO | NO | NO | NO | NO |
| Genomic | 1 | 113 | 239 | 340 | 464 | 616 | 796 |
| sequence | | | | | | | |
| Amino acid | 2 | 114 | 240 | 341 | 465 | 617 | 797 |
| sequence | | | | | | | |
| gRNA | 3-112 | 115-238 | 241-339 | 342-463 | 466-615 | 618-795 | 798-1024 |
| sequence | | | | | | | |

TABLE 2
Summary of Peanut (Arachis hypogaea) sequences within the scope of the present invention

| | AhSP1 | AhSP2 | AhSP3 | AhSP4 | AhSP5 | AhSP6 | AhSP7 | Ah SP8 | AhSP9 |
|---|---|---|---|---|---|---|---|---|---|
| Sequence | SEQ ID | SEQ ID | SEQ ID | SEQ ID | SEQ ID | SEQ ID | SEQ ID | SEQ ID | SEQ ID |
| type | NO | NO | NO | NO | NO | NO | NO | NO | NO |
| Genomic | 1025 | 1098 | 1180 | 1290 | 1409 | 1529 | 1640 | 1749 | 1928 |
| sequence | | | | | | | | | |
| Amino acid | 1026 | 1097 | 1181 | 1291 | 1410 | 1530 | 1641 | 1750 | 1929 |
| sequence | | | | | | | | | |
| gRNA | 1027- | 1100- | 1182- | 1292- | 1411- | 1531- | 1642- | 1751- | 1930- |
| sequence | 1097 | 1179 | 1289 | 1408 | 1528 | 1639 | 1748 | 1927 | 2051 |

The above gRNA molecules have been cloned into suitable vectors and their sequence has been verified. In addition different Cas9 versions have been analyzed for optimal compatibility between the Cas9 protein activity and the gRNA molecule in the Legume plant.

The efficiency of the designed gRNA molecules have been validated by transiently transforming Legume tissue culture. A plasmid carrying a gRNA sequence together with the Cas9 gene has been transformed into Legumes protoplasts. The protoplast cells have been grown for a short period of time and then were analyzed for existence of genome editing events. The positive constructs have been subjected to the herein established stable transformation protocol into Legume plant tissue for producing genome edited Legume plants in SP genes.

Stage 3: Transforming Legume plants using Agrobacterium or biolistics (gene gun) methods. For Agrobacterium and bioloistics, a DNA plasmid carrying (Cas9+gene specific gRNA) can be used. A vector containing a selection marker, Cas9 gene and relevant gene specific gRNA's is constructed. For biolistics, Ribonucleoprotein (RNP) complexes carrying (Cas9 protein+gene specific gRNA) are used. RNP complexes are created by mixing the Cas9 protein with relevant gene specific gRNA's.

According to some embodiments of the present invention, transformation of various Legume tissues was performed using particle bombardment of:
DNA vectors
Ribonucleoprotein complex (RNP's)

According to further embodiments of the present invention, transformation of various Legume tissues was performed using Agrobacterium (Agrobacterium tumefaciens) by:
Regeneration-based transformation
Floral-dip transformation
Seedling transformation

Transformation efficiency by A. tumefaciens has been compared to the bombardment method by transient GUS transformation experiment. After transformation, GUS staining of the transformants has been performed.

Screening for CRISPR/Cas9 gene editing events has been performed by at least one of the following analysis methods:
Restriction Fragment Length Polymorphism (RFLP)
Next Generation Sequencing (NGS)
PCR fragment analysis
Fluorescent-tag based screening
High resolution melting curve analysis (HRMA)

### REFERENCES

Tingdong Li, Xinping Yang, Yuan Yu, Xiaomin Si, Xiawan Zhai, Huawei Zhang, Wenxia Dong, Caixia Gao & Cao Xu. “Domestication of wild tomato is accelerated by genome editing” Nature Biotechnology 36(2018):1160-1163.
Agustin Zsögön, Tomás C̆ermák, Emmanuel Rezende Naves, Marcela Morato Notini, Kai H Edel, Stefan Weinl, Luciano Freschi, Daniel F Voytas, Jörg Kudla & Lázaro Eustáquio Pereira Peres. “De novo domestication of wild tomato using genome editing”. Nature Biotechnology 36(2018): 1211-1216.
Zachary H. Lemmon, Nathan T. Reem, Justin Dalrymple, Sebastian Soyk, Kerry E. Swartwood, Daniel Rodriguez-Leal, Joyce Van Eck & Zachary B. Lippman. “Rapid improvement of domestication traits in an orphan crop by genome editing”. Nature Plants 4(2018): 766-770.
Xie Kabin, and Yinong Yang. “RNA-guided genome editing in plants using a CRISPR-Cas system” Molecular plant 6.6 (2013): 1975-1983.

1. A method for producing a modified Legume plant exhibiting at least one improved domestication trait, wherein said method comprises steps of introducing by targeted genome editing a loss of function mutation in a Legume SELF PRUNING (SP) gene selected from Cowpea (Vigna unguiculata) SP gene or Peanut (Arachis hypogaea) SP gene, said improved domestication trait is relative to a corresponding Legume plant lacking the loss of function mutation.

2. The method according to claim 1, wherein said Cowpea (Vigna unguiculata) SP gene is selected from VuSP1-VuSP7 comprising a nucleic acid sequence with at least 75% sequence identity to a sequence selected from SEQ ID NO:1, SEQ ID NO:113, SEQ ID NO:239, SEQ ID NO:340, SEQ ID NO:464, SEQ ID NO:616 and SEQ ID NO:796, respectively, or a functional variant thereof and any combination thereof.

3. The method according to claim 1, wherein said Peanut (Arachis hypogaea) SP gene is selected from AhSP1-AhSP9 comprising a nucleic acid sequence with at least 75% sequence identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

4. The method according to claim 1, wherein said method comprises steps of:
a. identifying the Legume SP gene in a Cowpea (Vigna unguiculata) or Peanut (Arachis hypogaea) plant;
b. synthetizing at least one guide RNA (gRNA) comprising a nucleotide sequence complementary to said at least one identified Legume SP gene;
c. transforming the predetermined Legume plant cells with a construct comprising (a) Cas nucleotide sequence operably linked to said at least one gRNA, or (b) a ribonucleoprotein (RNP) complex comprising Cas protein and said at least one gRNA;
d. screening the genome of said transformed predetermined Legume plant cells for induced targeted loss of function mutation in said at least one Legume SP gene;
e. regenerating Legume plants carrying said loss of function mutation in at least one of said Legume SP gene; and
f. screening said regenerated plants for a Legume plant with improved domestication trait.

5. The method according to claim 4, wherein said step of screening the genome of said transformed plant cells for induced targeted loss of function mutation further comprises steps of obtaining a nucleic acid sample of said transformed plant and performing a nucleic acid amplification and optionally restriction enzyme digestion to detect a mutation in said at least one of said Legume SP gene.

6. The method according to claim 1, wherein said SP gene is selected from the group consisting of Cowpea SP genes comprising a sequence having at least 75% identity to a sequence selected from SEQ ID NO:1, SEQ ID NO:113, SEQ ID NO:239, SEQ ID NO:340, SEQ ID NO:464, SEQ ID NO:616 and SEQ ID NO:796, or Peanut SP gene comprising a sequence having at least 75% identity to a sequence selected from SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, or a functional variant thereof and any combination thereof.

7. The method according to claim 1, wherein said mutation is introduced using CRISPR (Clustered Regularly Interspaced Short Palindromic Repeats) and CRISPR-associated (Cas) gene (CRISPR/Cas), Transcription activator-like effector nuclease (TALEN), Zinc Finger Nuclease (ZFN), meganuclease or any combination thereof.

8. The method according to claim 7, wherein said Cas gene is selected from the group consisting of Cas9, Cas12, Cas13, Cas14, CasX, CasY, Csn1, Cpf1 and any combination thereof.

9. The method according to claim 1, wherein the mutated SP gene is a CRISPR/Cas9-induced heritable mutated allele.

10. The method according to claim 1, wherein said mutation is a missense mutation, nonsense mutation, insertion, deletion, indel, substitution or duplication.

11. The method according to claim 1, wherein said mutation is a silencing mutation, a knockdown mutation, a knockout mutation, a loss of function mutation or any combination thereof.

12. The method according to claim 1, wherein said mutation is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for said at least one Cowpea SP gene, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051, for said at least one Peanut SP gene, and any combination thereof.

13. The method according to claim 1, wherein said mutation in said Cowpea and Peanut SP genes is generated in planta via introduction of a construct comprising (a) Cas DNA and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively, or (b) a ribonucleoprotein (RNP) complex comprising Cas protein and gRNA sequence selected from the group consisting of SEQ ID NO:3-112, SEQ ID NO:115-238, SEQ ID NO:241-339, SEQ ID NO:342-463, SEQ ID NO:466-615, SEQ ID NO:618-795 and SEQ ID NO:798-1024 for Cowpea SEQ ID NO:1, SEQ ID NO:113, SEQ ID NO:239, SEQ ID NO:340, SEQ ID NO:464, SEQ ID NO:616 and SEQ ID NO:796, respectively, and SEQ ID NO:1027-1097, SEQ ID NO:1100-1179, SEQ ID NO:1182-1289, SEQ ID NO:1292-1408, SEQ ID NO:1411-1528, SEQ ID NO:1531-1639, SEQ ID NO:1642-1748, SEQ ID NO:1751-1927 and SEQ ID NO:1930-2051 for Peanut SEQ ID NO:1025, SEQ ID NO:1098, SEQ ID NO:1180, SEQ ID NO:1290, SEQ ID NO:1409, SEQ ID NO:1529, SEQ ID NO:1640, SEQ ID NO:1749 and SEQ ID NO:1928, respectively.

14. The method according to claim 12, wherein said gRNA sequence comprises a 3′ NGG Protospacer Adjacent Motif (PAM).

15. The method according to claim 12, wherein said construct is introduced into the plant cells via Agrobacterium infiltration, virus based plasmids for delivery of the genome editing molecules- or mechanical insertion such as polyethylene glycol (PEG) mediated DNA transformation, electroporation or gene gun biolistics.

16-52. (canceled)

53. The method according to claim 13, wherein said gRNA sequence comprises a 3′ NGG Protospacer Adjacent Motif (PAM).

54. The method according to claim 13, wherein said construct is introduced into the plant cells via Agrobacterium infiltration, virus based plasmids for delivery of the genome editing molecules or mechanical insertion such as polyethylene glycol (PEG) mediated DNA transformation, electroporation or gene gun biolistics.


SEQUENCE LISTING (2051 sequences)

SEQ ID NO: 1 - DNA, 1165 bp, Vigna unguiculata
ttcacacacagcaaagcagcaacattaactatttggttgcaaaagcttgaaagtaaagtgtagctagcattttaactaatctttccatggccatgaacatgatatcaacagatcctcttgtgattgggagggtgatcggagatgttgtcgatcctttcactccaaccgtcaaaatcaccgtctcctacaacaataagcaggtctataacggtcatgagtttttcccttcctcagtaaccaccaagcccaaggttcagattcgtggaggcgacatgagatccttcttcacccttgtaaccatcttctttccctccactctccttcatcacttcaaccttttcatgcatctcactcactcttccatttccatgcctttctttcagctcatgatagatccagacgttcctggccccagtgatccatatctcagggaacacttacactggtatcatactaaaccatcacatgcagagtttacttggtttatgatttcatctgaaacaatgtgtctgaccatatttaacaactcacaggatagtcacagacatccctggcacaacggacaccacatttggtaagttatataatcttattacagacacataactttcataaaaatctgtgttttttttgttttgaacatgcaatggaacaggaaatgaggtggtgaactatgaaatcccaaggccaaacataggcatccataggtttgtgttcttccttttcaagcagaagtgcaggcaggcagtgatgaaaataccaagttctagggacctcttcaacaccaggacctttgcagaggacaatgaccttggccttcctgtggctgctgtgtttttcaatgctcaaagggaaactgctgccagaagacgttgaataatgaaccaacccacaaaagtgccacaatgtgcttatcactactaaagcctacatacctgtccactgcagtagtgcccaccaaataagtctaggtttctaagggtttgtggggcactttgtctagttaaattccaacttgcatgctgaaactctcgtagcatctaacattcttgtttctagtttcgtgttttcagtttgtaaccatttcagtttctgtttaactttctcgtaatctatgtctatgtcccagtaaccatgcaagttattattacattaactagtttta

SEQ ID NO: 2 - protein, 176 aa, Vigna unguiculata
MAMNMISTDPLVIGRVIGDVVDPFTPTVKITVSYNNKQVYNGHEFFPSSVTTKPKVQIRGGDMRSFFTLLMIDPDVPGPSDPYLREHLHWIVTDIPGTTDTTFGNEVVNYEIPRPNIGIHRFVFFLFKQKCRQAVMKIPSSRDLFNTRTFAEDNDLGLPVAAVFFNAQRETAARRR

SEQ ID NO: 3 - DNA, 20 bp, Vigna_unguiculata
aagcagcaacattaactatt

SEQ ID NO: 4 - DNA, 20 bp, Vigna_unguiculata
cattttaactaatctttcca

SEQ ID NO: 5 - DNA, 20 bp, Vigna_unguiculata
tgatatcatgttcatggcca

SEQ ID NO: 6 - DNA, 20 bp, Vigna_unguiculata
atctgttgatatcatgttca

SEQ ID NO: 7 - DNA, 20 bp, Vigna_unguiculata
atcaccctcccaatcacaag

SEQ ID NO: 8 - DNA, 20 bp, Vigna_unguiculata
tcaacagatcctcttgtgat

SEQ ID NO: 9 - DNA, 20 bp, Vigna_unguiculata
caacagatcctcttgtgatt

SEQ ID NO: 10 - DNA, 20 bp, Vigna_unguiculata
cagatcctcttgtgattggg

SEQ ID NO: 11 - DNA, 20 bp, Vigna_unguiculata
agatcctcttgtgattggga

SEQ ID NO: 12 - DNA, 20 bp, Vigna_unguiculata
cttgtgattgggagggtgat

SEQ ID NO: 13 - DNA, 20 bp, Vigna_unguiculata
attttgacggttggagtgaa

SEQ ID NO: 14 - DNA, 20 bp, Vigna_unguiculata
gagacggtgattttgacggt

SEQ ID NO: 15 - DNA, 20 bp, Vigna_unguiculata
gtaggagacggtgattttga

SEQ ID NO: 16 - DNA, 20 bp, Vigna_unguiculata
ctgcttattgttgtaggaga

SEQ ID NO: 17 - DNA, 20 bp, Vigna_unguiculata
atagacctgcttattgttgt

SEQ ID NO: 18 - DNA, 20 bp, Vigna_unguiculata
cgtctcctacaacaataagc

SEQ ID NO: 19 - DNA, 20 bp, Vigna_unguiculata
aacaataagcaggtctataa

SEQ ID NO: 20 - DNA, 20 bp, Vigna_unguiculata
gcttggtggttactgaggaa

SEQ ID NO: 21 - DNA, 20 bp, Vigna_unguiculata
ggcttggtggttactgagga

SEQ ID NO: 22 - DNA, 20 bp, Vigna_unguiculata
cttgggcttggtggttactg

SEQ ID NO: 23 - DNA, 20 bp, Vigna_unguiculata
aatctgaaccttgggcttgg

SEQ ID NO: 24 - DNA, 20 bp, Vigna_unguiculata
ctcagtaaccaccaagccca

SEQ ID NO: 25 - DNA, 20 bp, Vigna_unguiculata
acgaatctgaaccttgggct

SEQ ID NO: 26 - DNA, 20 bp, Vigna_unguiculata
cctccacgaatctgaacctt

SEQ ID NO: 27 - DNA, 20 bp, Vigna_unguiculata
gcctccacgaatctgaacct

SEQ ID NO: 28 - DNA, 20 bp, Vigna_unguiculata
aagcccaaggttcagattcg

SEQ ID NO: 29 - DNA, 20 bp, Vigna_unguiculata
cccaaggttcagattcgtgg

SEQ ID NO: 30 - DNA, 20 bp, Vigna_unguiculata
gatggttacaagggtgaaga

SEQ ID NO: 31 - DNA, 20 bp, Vigna_unguiculata
gggaaagaagatggttacaa

SEQ ID NO: 32 - DNA, 20 bp, Vigna_unguiculata
agggaaagaagatggttaca

SEQ ID NO: 33 - DNA, 20 bp, Vigna_unguiculata
gagagtggagggaaagaaga

SEQ ID NO: 34 - DNA, 20 bp, Vigna_unguiculata
aagtgatgaaggagagtgga

SEQ ID NO: 35 - DNA, 20 bp, Vigna_unguiculata
gaagtgatgaaggagagtgg

SEQ ID NO: 36 - DNA, 20 bp, Vigna_unguiculata
gttgaagtgatgaaggagag

SEQ ID NO: 37 - DNA, 20 bp, Vigna_unguiculata
tgaaaaggttgaagtgatga

SEQ ID NO: 38 - DNA, 20 bp, Vigna_unguiculata
agtgagtgagatgcatgaaa

SEQ ID NO: 39 - DNA, 20 bp, Vigna_unguiculata
ctgaaagaaaggcatggaaa

SEQ ID NO: 40 - DNA, 20 bp, Vigna_unguiculata
catgagctgaaagaaaggca

SEQ ID NO: 41 - DNA, 20 bp, Vigna_unguiculata
tctatcatgagctgaaagaa

SEQ ID NO: 42 - DNA, 20 bp, Vigna_unguiculata
tcactggggccaggaacgtc

SEQ ID NO: 43 - DNA, 20 bp, Vigna_unguiculata
atgatagatccagacgttcc

SEQ ID NO: 44 - DNA, 20 bp, Vigna_unguiculata
agatatggatcactggggcc

SEQ ID NO: 45 - DNA, 20 bp, Vigna_unguiculata
ccctgagatatggatcactg

SEQ ID NO: 46 - DNA, 20 bp, Vigna_unguiculata
tccctgagatatggatcact

SEQ ID NO: 47 - DNA, 20 bp, Vigna_unguiculata
ttccctgagatatggatcac

SEQ ID NO: 48 - DNA, 20 bp, Vigna_unguiculata
gccccagtgatccatatctc

SEQ ID NO: 49 - DNA, 20 bp, Vigna_unguiculata
tgtaagtgttccctgagata

SEQ ID NO: 50 - DNA, 20 bp, Vigna_unguiculata
ccccagtgatccatatctca

SEQ ID NO: 51 - DNA, 20 bp, Vigna_unguiculata
atctcagggaacacttacac

SEQ ID NO: 52 - DNA, 20 bp, Vigna_unguiculata
caagtaaactctgcatgtga

SEQ ID NO: 53 - DNA, 20 bp, Vigna_unguiculata
catcacatgcagagtttact

SEQ ID NO: 54 - DNA, 20 bp, Vigna_unguiculata
atcctgtgagttgttaaata

SEQ ID NO: 55 - DNA, 20 bp, Vigna_unguiculata
gaccatatttaacaactcac

SEQ ID NO: 56 - DNA, 20 bp, Vigna_unguiculata
aggatagtcacagacatccc

SEQ ID NO: 57 - DNA, 20 bp, Vigna_unguiculata
atgtggtgtccgttgtgcca

SEQ ID NO: 58 - DNA, 20 bp, Vigna_unguiculata
aatgtggtgtccgttgtgcc

SEQ ID NO: 59 - DNA, 20 bp, Vigna_unguiculata
cacagacatccctggcacaa

SEQ ID NO: 60 - DNA, 20 bp, Vigna_unguiculata
ggcacaacggacaccacatt

SEQ ID NO: 61 - DNA, 20 bp, Vigna_unguiculata
attatataacttaccaaatg

SEQ ID NO: 62 - DNA, 20 bp, Vigna_unguiculata
tttttgttttgaacatgcaa

SEQ ID NO: 63 - DNA, 20 bp, Vigna_unguiculata
ttttgaacatgcaatggaac

SEQ ID NO: 64 - DNA, 20 bp, Vigna_unguiculata
atgcaatggaacaggaaatg

SEQ ID NO: 65 - DNA, 20 bp, Vigna_unguiculata
caatggaacaggaaatgagg

SEQ ID NO: 66 - DNA, 20 bp, Vigna_unguiculata
tggtgaactatgaaatccca

SEQ ID NO: 67 - DNA, 20 bp, Vigna_unguiculata
ggatgcctatgtttggcctt

SEQ ID NO: 68 - DNA, 20 bp, Vigna_unguiculata
tggatgcctatgtttggcct

SEQ ID NO: 69 - DNA, 20 bp, Vigna_unguiculata
gaaatcccaaggccaaacat

SEQ ID NO: 70 - DNA, 20 bp, Vigna_unguiculata
aacctatggatgcctatgtt

SEQ ID NO: 71 - DNA, 20 bp, Vigna_unguiculata
ggccaaacataggcatccat

SEQ ID NO: 72 - DNA, 20 bp, Vigna_unguiculata
aaaggaagaacacaaaccta

SEQ ID NO: 73 - DNA, 20 bp, Vigna_unguiculata
gcctgcacttctgcttgaaa

SEQ ID NO: 74 - DNA, 20 bp, Vigna_unguiculata
tccttttcaagcagaagtgc

SEQ ID NO: 75 - DNA, 20 bp, Vigna_unguiculata
tttcaagcagaagtgcaggc

SEQ ID NO: 76 - DNA, 20 bp, Vigna_unguiculata
tgatgaaaataccaagttct

SEQ ID NO: 77 - DNA, 20 bp, Vigna_unguiculata
ttgaagaggtccctagaact

SEQ ID NO: 78 - DNA, 20 bp, Vigna_unguiculata
gatgaaaataccaagttcta

SEQ ID NO: 79 - DNA, 20 bp, Vigna_unguiculata
caaaggtcctggtgttgaag

SEQ ID NO: 80 - DNA, 20 bp, Vigna_unguiculata
ctagggacctcttcaacacc

SEQ ID NO: 81 - DNA, 20 bp, Vigna_unguiculata
attgtcctctgcaaaggtcc

SEQ ID NO: 82 - DNA, 20 bp, Vigna_unguiculata
caacaccaggacctttgcag

SEQ ID NO: 83 - DNA, 20 bp, Vigna_unguiculata
aaggtcattgtcctctgcaa

SEQ ID NO: 84 - DNA, 20 bp, Vigna_unguiculata
tttgcagaggacaatgacct

SEQ ID NO: 85 - DNA, 20 bp, Vigna_unguiculata
cagcagccacaggaaggcca

SEQ ID NO: 86 - DNA, 20 bp, Vigna_unguiculata
caatgaccttggccttcctg

SEQ ID NO: 87 - DNA, 20 bp, Vigna_unguiculata
aaaacacagcagccacagga

SEQ ID NO: 88 - DNA, 20 bp, Vigna_unguiculata
ttgaaaaacacagcagccac

SEQ ID NO: 89 - DNA, 20 bp, Vigna_unguiculata
ctgtgtttttcaatgctcaa

SEQ ID NO: 90 - DNA, 20 bp, Vigna_unguiculata
tgtgtttttcaatgctcaaa

SEQ ID NO: 91 - DNA, 20 bp, Vigna_unguiculata
gttcattattcaacgtcttc

SEQ ID NO: 92 - DNA, 20 bp, Vigna_unguiculata
attgtggcacttttgtgggt

SEQ ID NO: 93 - DNA, 20 bp, Vigna_unguiculata
gcacattgtggcacttttgt

SEQ ID NO: 94 - DNA, 20 bp, Vigna_unguiculata
agcacattgtggcacttttg

SEQ ID NO: 95 - DNA, 20 bp, Vigna_unguiculata
tagtagtgataagcacattg

SEQ ID NO: 96 - DNA, 20 bp, Vigna_unguiculata
actgcagtggacaggtatgt

SEQ ID NO: 97 - DNA, 20 bp, Vigna_unguiculata
tgggcactactgcagtggac

SEQ ID NO: 98 - DNA, 20 bp, Vigna_unguiculata
tttggtgggcactactgcag

SEQ ID NO: 99 - DNA, 20 bp, Vigna_unguiculata
gaaacctagacttatttggt

SEQ ID NO: 100 - DNA, 20 bp, Vigna_unguiculata
agaaacctagacttatttgg

SEQ ID NO: 101 - DNA, 20 bp, Vigna_unguiculata
cttagaaacctagacttatt

SEQ ID NO: 102 - DNA, 20 bp, Vigna_unguiculata
agtgcccaccaaataagtct

SEQ ID NO: 103 - DNA, 20 bp, Vigna_unguiculata
caaataagtctaggtttcta

SEQ ID NO: 104 - DNA, 20 bp, Vigna_unguiculata
aaataagtctaggtttctaa

SEQ ID NO: 105 - DNA, 20 bp, Vigna_unguiculata
tctaggtttctaagggtttg

SEQ ID NO: 106 - DNA, 20 bp, Vigna_unguiculata
ctaggtttctaagggtttgt

SEQ ID NO: 107 - DNA, 20 bp, Vigna_unguiculata
taggtttctaagggtttgtg

SEQ ID NO: 108 - DNA, 20 bp, Vigna_unguiculata
gagagtttcagcatgcaagt

SEQ ID NO: 109 - DNA, 20 bp, Vigna_unguiculata
aagttaaacagaaactgaaa

SEQ ID NO: 110 - DNA, 20 bp, Vigna_unguiculata
taataacttgcatggttact

SEQ ID NO: 111 - DNA, 20 bp, Vigna_unguiculata
ataataacttgcatggttac

SEQ ID NO: 112 - DNA, 20 bp, Vigna_unguiculata
taatgtaataataacttgca

SEQ ID NO: 113 - DNA, 1708 bp, Vigna unguiculata
cacacttggtttctgagcaggactatcaagttgtgttcttgtgttcttcagttttcactctctaaactttgttctcttacccttatttgcaatggcaagaatgcctttagaacctcttatagtggggagagtcataggagaggttcttgactctttcaccacaagcacaaaaatgactgtgagttataacaaaaagcaagtctacaatggccatgagtttttcccttcatctatcaacatcaaacccaaggttgagattgagggtggtgatatgagatcctttttcacactggtatctattttttttccttctctcttcttcttcctctttttctttaaacaccaaattaaagcttttaaaaaaggaaaataatgcttcagctgttatttgttgtcatatatatctttaatcatgctcacgcattatgtctcttttaatgaaagtttgccctttacaaaacaaacactgaatcattaaactaatgtctcccttttttggcgcagatcatgacagaccctgatgttccaggccctagtgacccttatctgagagaacacttgcactggtatacttaacacaaagataaactttgtttaaacttaaacccacccaccctaaacaacttaaatgaaaacattttgaaagtaatgaaccggttatataaccatccgacattcttgttagatgtgtgtgtctttgatcaagtttaaaaattttgcaagacatatgcattcctttcatcgcatcaaacaacagaggtaacaaacatgtatacatatacatatacatatacatacctttgtgtatatattctgtaaaagttgtaaggtgttgcagctaccatcgttttcctccacccaccctaaaaccaaaaacagttttaaagggtgaataaatctcggtgactgaacaacaaacataacagaactgcataggagatataaggtaaagtagcgattcttaggtatctaacagacaataattttgttttttgcaggatagtgacagacattccaggcacaacagatgctacatttggtaggttgatgtaaatgattgattagaaggaaaaatgtgggaacttacttgctgtgtgtgtggtgatcggatgcatgatgagagagactgacaaatgaaaaatgatgtttttttcttttgtagggaaagagttggtgagctatgagatcccaaaacctaatattgggattcataggtttgtgtttgtcctgttcaagcaaaagcgtagacagtgtgttactccacctagttcaagggatcacttcaacacacgcaatttcgcagcacagaacgagcttggcctcccagtggctgctgtctacttcaatgcacagagggaaacggctgcaagaagacgctagcttcctatacctatagttcctatagctatagcttctgctcctgcttgctcttatttaccactgcaaccaccaaagtagtagtttgaataaagccaaaagtacaacgagtttcagtactgcaacttcttaatcctcctcacatgtgcctcaaataactcgctcaaccaagtgaatcattttctagtttgactagtgtgtgtgctatgtttttactttcccatccaatgtgtaccttctgtattatatagtgtactacgttgtattattattacctcctggatactcctatgaggtgatcttatataaaccaataagaggtttggtgctc

SEQ ID NO: 114 - protein, 173 aa, Vigna unguiculata
MARMPLEPLIVGRVIGEVLDSFTTSTKMTVSYNKKQVYNGHEFFPSSINIKPKVEIEGGDMRSFFTLIMTDPDVPGPSDPYLREHLHWIVTDIPGTTDATFGKELVSYEIPKPNIGIHRFVFVLFKQKRRQCVTPPSSRDHFNTRNFAAQNELGLPVAAVYFNAQRETAARRR

SEQ ID NO: 115 - DNA, 20 bp, Vigna_unguiculata
attcttgccattgcaaataa

SEQ ID NO: 116 - DNA, 20 bp, Vigna_unguiculata
cattcttgccattgcaaata

SEQ ID NO: 117 - DNA, 20 bp, Vigna_unguiculata
tctcttacccttatttgcaa

SEQ ID NO: 118 - DNA, 20 bp, Vigna_unguiculata
cccactataagaggttctaa

SEQ ID NO: 119 - DNA, 20 bp, Vigna_unguiculata
atgactctccccactataag

SEQ ID NO: 120 - DNA, 20 bp, Vigna_unguiculata
gcctttagaacctcttatag

SEQ ID NO: 121 - DNA, 20 bp, Vigna_unguiculata
cctttagaacctcttatagt

SEQ ID NO: 122 - DNA, 20 bp, Vigna_unguiculata
ctttagaacctcttatagtg

SEQ ID NO: 123 - DNA, 20 bp, Vigna_unguiculata
cttatagtggggagagtcat

SEQ ID NO: 124 - DNA, 20 bp, Vigna_unguiculata
agtggggagagtcataggag

SEQ ID NO: 125 - DNA, 20 bp, Vigna_unguiculata
cacagtcatttttgtgcttg

SEQ ID NO: 126 - DNA, 20 bp, Vigna_unguiculata
aacaaaaagcaagtctacaa

SEQ ID NO: 127 - DNA, 20 bp, Vigna_unguiculata
tagatgaagggaaaaactca

SEQ ID NO: 128 - DNA, 20 bp, Vigna_unguiculata
gtttgatgttgatagatgaa

SEQ ID NO: 129 - DNA, 20 bp, Vigna_unguiculata
ggtttgatgttgatagatga

SEQ ID NO: 130 - DNA, 20 bp, Vigna_unguiculata
atctatcaacatcaaaccca

SEQ ID NO: 131 - DNA, 20 bp, Vigna_unguiculata
ccaccctcaatctcaacctt

SEQ ID NO: 132 - DNA, 20 bp, Vigna_unguiculata
accaccctcaatctcaacct

SEQ ID NO: 133 - DNA, 20 bp, Vigna_unguiculata
caaacccaaggttgagattg

SEQ ID NO: 134 - DNA, 20 bp, Vigna_unguiculata
aaacccaaggttgagattga

SEQ ID NO: 135 - DNA, 20 bp, Vigna_unguiculata
cccaaggttgagattgaggg

SEQ ID NO: 136 - DNA, 20 bp, Vigna_unguiculata
aatagataccagtgtgaaaa

SEQ ID NO: 137 - DNA, 20 bp, Vigna_unguiculata
tatgagatcctttttcacac

SEQ ID NO: 138 - DNA, 20 bp, Vigna_unguiculata
aagaggaagaagaagagaga

SEQ ID NO: 139 - DNA, 20 bp, Vigna_unguiculata
tttggtgtttaaagaaaaag

SEQ ID NO: 140 - DNA, 20 bp, Vigna_unguiculata
cttttttaaaagctttaatt

SEQ ID NO: 141 - DNA, 20 bp, Vigna_unguiculata
caaattaaagcttttaaaaa

SEQ ID NO: 142 - DNA, 20 bp, Vigna_unguiculata
ttcagtgtttgttttgtaaa

SEQ ID NO: 143 - DNA, 20 bp, Vigna_unguiculata
attcagtgtttgttttgtaa

SEQ ID NO: 144 - DNA, 20 bp, Vigna_unguiculata
aaactaatgtctcccttttt

SEQ ID NO: 145 - DNA, 20 bp, Vigna_unguiculata
tcatgatctgcgccaaaaaa

SEQ ID NO: 146 - DNA, 20 bp, Vigna_unguiculata
gtcatgatctgcgccaaaaa

SEQ ID NO: 147 - DNA, 20 bp, Vigna_unguiculata
cactagggcctggaacatca

SEQ ID NO: 148 - DNA, 20 bp, Vigna_unguiculata
tcactagggcctggaacatc

SEQ ID NO: 149 - DNA, 20 bp, Vigna_unguiculata
atgacagaccctgatgttcc

SEQ ID NO: 150 - DNA, 20 bp, Vigna_unguiculata
agataagggtcactagggcc

SEQ ID NO: 151 - DNA, 20 bp, Vigna_unguiculata
ctctcagataagggtcacta

SEQ ID NO: 152 - DNA, 20 bp, Vigna_unguiculata
tctctcagataagggtcact

SEQ ID NO: 153 - DNA, 20 bp, Vigna_unguiculata
gcaagtgttctctcagataa

SEQ ID NO: 154 - DNA, 20 bp, Vigna_unguiculata
tgcaagtgttctctcagata

SEQ ID NO: 155 - DNA, 20 bp, Vigna_unguiculata
atctgagagaacacttgcac

SEQ ID NO: 156 - DNA, 20 bp, Vigna_unguiculata
ttaagttgtttagggtgggt

SEQ ID NO: 157 - DNA, 20 bp, Vigna_unguiculata
tttaagttgtttagggtggg

SEQ ID NO: 158 - DNA, 20 bp, Vigna_unguiculata
tcatttaagttgtttagggt

SEQ ID NO: 159 - DNA, 20 bp, Vigna_unguiculata
ttcatttaagttgtttaggg

SEQ ID NO: 160 - DNA, 20 bp, Vigna_unguiculata
gttttcatttaagttgttta

SEQ ID NO: 161 - DNA, 20 bp, Vigna_unguiculata
tgttttcatttaagttgttt

SEQ ID NO: 162 - DNA, 20 bp, Vigna_unguiculata
acattttgaaagtaatgaac

SEQ ID NO: 163 - DNA, 20 bp, Vigna_unguiculata
atgtcggatggttatataac

SEQ ID NO: 164 - DNA, 20 bp, Vigna_unguiculata
catctaacaagaatgtcgga

SEQ ID NO: 165 - DNA, 20 bp, Vigna_unguiculata
cacacatctaacaagaatgt

SEQ ID NO: 166 - DNA, 20 bp, Vigna_unguiculata
ctgttgtttgatgcgatgaa

SEQ ID NO: 167 - DNA, 20 bp, Vigna_unguiculata
ttcatcgcatcaaacaacag

SEQ ID NO: 168 - DNA, 20 bp, Vigna_unguiculata
tttacagaatatatacacaa

SEQ ID NO: 169 - DNA, 20 bp, Vigna_unguiculata
tatattctgtaaaagttgta

SEQ ID NO: 170 - DNA, 20 bp, Vigna_unguiculata
gggtgggtggaggaaaacga

SEQ ID NO: 171 - DNA, 20 bp, Vigna_unguiculata
tttggttttagggtgggtgg

SEQ ID NO: 172 - DNA, 20 bp, Vigna_unguiculata
gtttttggttttagggtggg

SEQ ID NO: 173 - DNA, 20 bp, Vigna_unguiculata
actgtttttggttttagggt

SEQ ID NO: 174 - DNA, 20 bp, Vigna_unguiculata
aactgtttttggttttaggg

SEQ ID NO: 175 - DNA, 20 bp, Vigna_unguiculata
taaaactgtttttggtttta

SEQ ID NO: 176 - DNA, 20 bp, Vigna_unguiculata
ttaaaactgtttttggtttt

SEQ ID NO: 177 - DNA, 20 bp, Vigna_unguiculata
tcaccctttaaaactgtttt

SEQ ID NO: 178 - DNA, 20 bp, Vigna_unguiculata
aaaaccaaaaacagttttaa

SEQ ID NO: 179 - DNA, 20 bp, Vigna_unguiculata
aaaccaaaaacagttttaaa

SEQ ID NO: 180 - DNA, 20 bp, Vigna_unguiculata
tttaaagggtgaataaatct

SEQ ID NO: 181 - DNA, 20 bp, Vigna_unguiculata
caaacataacagaactgcat

SEQ ID NO: 182 - DNA, 20 bp, Vigna_unguiculata
agaactgcataggagatata

SEQ ID NO: 183 - DNA, 20 bp, Vigna_unguiculata
aaggtaaagtagcgattctt

SEQ ID NO: 184 - DNA, 20 bp, Vigna_unguiculata
acaataattttgttttttgc

SEQ ID NO: 185 - DNA, 20 bp, Vigna_unguiculata
aggatagtgacagacattcc

SEQ ID NO: 186 - DNA, 20 bp, Vigna_unguiculata
aatgtagcatctgttgtgcc

SEQ ID NO: 187 - DNA, 20 bp, Vigna_unguiculata
ggcacaacagatgctacatt

SEQ ID NO: 188 - DNA, 20 bp, Vigna_unguiculata
caacagatgctacatttggt

SEQ ID NO: 189 - DNA, 20 bp, Vigna_unguiculata
gatgtaaatgattgattaga

SEQ ID NO: 190 - DNA, 20 bp, Vigna_unguiculata
attgattagaaggaaaaatg

SEQ ID NO: 191 - DNA, 20 bp, Vigna_unguiculata
ttgattagaaggaaaaatgt

SEQ ID NO: 192 - DNA, 20 bp, Vigna_unguiculata
gaacttacttgctgtgtgtg

SEQ ID NO: 193 - DNA, 20 bp, Vigna_unguiculata
cttgctgtgtgtgtggtgat

SEQ ID NO: 194 - DNA, 20 bp, Vigna_unguiculata
atgatgtttttttcttttgt

SEQ ID NO: 195 - DNA, 20 bp, Vigna_unguiculata
tgatgtttttttcttttgta

SEQ ID NO: 196 - DNA, 20 bp, Vigna_unguiculata
ttcttttgtagggaaagagt

SEQ ID NO: 197 - DNA, 20 bp, Vigna_unguiculata
gaatcccaatattaggtttt

SEQ ID NO: 198 - DNA, 20 bp, Vigna_unguiculata
tgaatcccaatattaggttt

SEQ ID NO: 199 - DNA, 20 bp, Vigna_unguiculata
gagatcccaaaacctaatat

SEQ ID NO: 200 - DNA, 20 bp, Vigna_unguiculata
agatcccaaaacctaatatt

SEQ ID NO: 201 - DNA, 20 bp, Vigna_unguiculata
aacctatgaatcccaatatt

SEQ ID NO: 202 - DNA, 20 bp, Vigna_unguiculata
aacctaatattgggattcat

SEQ ID NO: 203 - DNA, 20 bp, Vigna_unguiculata
gtctacgcttttgcttgaac

SEQ ID NO: 204 - DNA, 20 bp, Vigna_unguiculata
aagtgatcccttgaactagg

SEQ ID NO: 205 - DNA, 20 bp, Vigna_unguiculata
gtgttactccacctagttca

SEQ ID NO: 206 - DNA, 20 bp, Vigna_unguiculata
ttgaagtgatcccttgaact

SEQ ID NO: 207 - DNA, 20 bp, Vigna_unguiculata
tgttactccacctagttcaa

SEQ ID NO: 208 - DNA, 20 bp, Vigna_unguiculata
ttcgcagcacagaacgagct

SEQ ID NO: 209 - DNA, 20 bp, Vigna_unguiculata
gaacgagcttggcctcccag

SEQ ID NO: 210 - DNA, 20 bp, Vigna_unguiculata
agtagacagcagccactggg

SEQ ID NO: 211 - DNA, 20 bp, Vigna_unguiculata
tgaagtagacagcagccact

SEQ ID NO: 212 - DNA, 20 bp, Vigna_unguiculata
ttgaagtagacagcagccac

SEQ ID NO: 213 - DNA, 20 bp, Vigna_unguiculata
ctgtctacttcaatgcacag

SEQ ID NO: 214 - DNA, 20 bp, Vigna_unguiculata
tgtctacttcaatgcacaga

SEQ ID NO: 215 - DNA, 20 bp, Vigna_unguiculata
cttcaatgcacagagggaaa

SEQ ID NO: 216 - DNA, 20 bp, Vigna_unguiculata
gctataggaactataggtat

SEQ ID NO: 217 - DNA, 20 bp, Vigna_unguiculata
gctatagctataggaactat

SEQ ID NO: 218 - DNA, 20 bp, Vigna_unguiculata
ggagcagaagctatagctat

SEQ ID NO: 219 - DNA, 20 bp, Vigna_unguiculata
agtggtaaataagagcaagc

SEQ ID NO: 220 - DNA, 20 bp, Vigna_unguiculata
tactactttggtggttgcag

SEQ ID NO: 221 - DNA, 20 bp, Vigna_unguiculata
tattcaaactactactttgg

SEQ ID NO: 222 - DNA, 20 bp, Vigna_unguiculata
ctttattcaaactactactt

SEQ ID NO: 223 - DNA, 20 bp, Vigna_unguiculata
actgaaactcgttgtacttt

SEQ ID NO: 224 - DNA, 20 bp, Vigna_unguiculata
tatttgaggcacatgtgagg

SEQ ID NO: 225 - DNA, 20 bp, Vigna_unguiculata
agttatttgaggcacatgtg

SEQ ID NO: 226 - DNA, 20 bp, Vigna_unguiculata
ttggttgagcgagttatttg

SEQ ID NO: 227 - DNA, 20 bp, Vigna_unguiculata
aaactagaaaatgattcact

SEQ ID NO: 228 - DNA, 20 bp, Vigna_unguiculata
acagaaggtacacattggat

SEQ ID NO: 229 - DNA, 20 bp, Vigna_unguiculata
tacagaaggtacacattgga

SEQ ID NO: 230 - DNA, 20 bp, Vigna_unguiculata
ataatacagaaggtacacat

SEQ ID NO: 231 - DNA, 20 bp, Vigna_unguiculata
agtacactatataatacaga

SEQ ID NO: 232 - DNA, 20 bp, Vigna_unguiculata
gttgtattattattacctcc

SEQ ID NO: 233 - DNA, 20 bp, Vigna_unguiculata
acctcataggagtatccagg

SEQ ID NO: 234 - DNA, 20 bp, Vigna_unguiculata
atcacctcataggagtatcc

SEQ ID NO: 235 - DNA, 20 bp, Vigna_unguiculata
acctcctggatactcctatg

SEQ ID NO: 236 - DNA, 20 bp, Vigna_unguiculata
tttatataagatcacctcat

SEQ ID NO: 237 - DNA, 20 bp, Vigna_unguiculata
atcttatataaaccaataag

SEQ ID NO: 238 - DNA, 20 bp, Vigna_unguiculata
atataaaccaataagaggtt

SEQ ID NO: 239 - DNA, 1261 bp, Vigna unguiculata
gttttttttttttccattcttctgtttcctccttcaccttgttgctgcaaccatggcaagagtatccacagatccactggttattgggagagtgataggagatgttcttgattctttcactccaaccataaaaatgactgtaacattctgtaagaagcaagtctacaatggccatgaactctttccttccacagttaccacatggcccagggttgagattggtggaggagatttgagatccttctttacactggtatatatatttatttccttttatttattgaacaaaaaagaactttatatatatttctttcaaaacttcaatcatgtaggcatgaaaaaaaaaccttcaatcatttgaaataaatgtctcctttttctctttgcagattatgacagaccctgatgtcccaggacctagtgacccttatctgagagaacatctgcactggtactttaacagatattgcctgaaattaaccgtttgtatttttcttcatttattggctgaataatatatttacctgaaaaccaaaccaattctaaactgagagaaggaaaaactctgaaacagttataaatattttaaaacatcaaattttgacattgaaaatcaattctatgcaccaaattggttttccatgaagggttataaaagttctaagttctaaccaaagacctaattttactgtgtgtggataaatacacacacacacacactactgtgcaaatcaaagcaaagaaattttcaggtttcagctgaaattgtgcccttttgcaggatggtgacagacataccaggcacaacaaatgcttcatttggtaactttatatttatttaagatgtactaaaaaaaatgtatgatcagaaatatacttaatccatgtttttgttccgttctttttggggtagggaatgtgttggttagctatgaaatgccaaagcctaacatagggatacacaggtttgtgtttgtcctgttccagcaaaaacgtaggcagtgtgttactccaccttcttcgagggacaactttaacactcgaaaattttcatccgagaacgaccttggactccctgttgctgctgtctacttcaatgcacagagggaaactgctgctagaagacgctaatatatatggctacttctgcaatgcaaccaatgtactcaataacgttatcttcacgtaggggcctagtttcacttctaaaccatcgtgtcgcttgaaatagctcgcagaaatgttttcttttttagtaact

SEQ ID NO: 240 - protein, 173 aa, Vigna unguiculata
MARVSTDPLVIGRVIGDVLDSFTPTIKMTVTFCKKQVYNGHELFPSTVTTWPRVEIGGGDLRSFFTLIMTDPDVPGPSDPYLREHLHWMVTDIPGTTNASFGNVLVSYEMPKPNIGIHRFVFVLFQQKRRQCVTPPSSRDNFNTRKFSSENDLGLPVAAVYFNAQRETAARRR

SEQ ID NO: 241 - DNA, 20 bp, Vigna_unguiculata
tgaaggaggaaacagaagaa

SEQ ID NO: 242 - DNA, 20 bp, Vigna_unguiculata
ttgcagcaacaaggtgaagg

SEQ ID NO: 243 - DNA, 20 bp, Vigna_unguiculata
tggttgcagcaacaaggtga

SEQ ID NO: 244 - DNA, 20 bp, Vigna_unguiculata
ttgccatggttgcagcaaca

SEQ ID NO: 245 - DNA, 20 bp, Vigna_unguiculata
tcaccttgttgctgcaacca

SEQ ID NO: 246 - DNA, 20 bp, Vigna_unguiculata
atctgtggatactcttgcca

SEQ ID NO: 247 - DNA, 20 bp, Vigna_unguiculata
cccaataaccagtggatctg

SEQ ID NO: 248 - DNA, 20 bp, Vigna_unguiculata
aagagtatccacagatccac

SEQ ID NO: 249 - DNA, 20 bp, Vigna_unguiculata
atcactctcccaataaccag

SEQ ID NO: 250 - DNA, 20 bp, Vigna_unguiculata
tccacagatccactggttat

SEQ ID NO: 251 - DNA, 20 bp, Vigna_unguiculata
ccacagatccactggttatt

SEQ ID NO: 252 - DNA, 20 bp, Vigna_unguiculata
ctggttattgggagagtgat

SEQ ID NO: 253 - DNA, 20 bp, Vigna_unguiculata
gttacagtcatttttatggt

SEQ ID NO: 254 - DNA, 20 bp, Vigna_unguiculata
gaatgttacagtcattttta

SEQ ID NO: 255 - DNA, 20 bp, Vigna_unguiculata
tgtaagaagcaagtctacaa

SEQ ID NO: 256 - DNA, 20 bp, Vigna_unguiculata
ctgtggaaggaaagagttca

SEQ ID NO: 257 - DNA, 20 bp, Vigna_unguiculata
ggccatgtggtaactgtgga

SEQ ID NO: 258 - DNA, 20 bp, Vigna_unguiculata
cctgggccatgtggtaactg

SEQ ID NO: 259 - DNA, 20 bp, Vigna_unguiculata
ttccttccacagttaccaca

SEQ ID NO: 260 - DNA, 20 bp, Vigna_unguiculata
aatctcaaccctgggccatg

SEQ ID NO: 261 - DNA, 20 bp, Vigna_unguiculata
ccacagttaccacatggccc

SEQ ID NO: 262 - DNA, 20 bp, Vigna_unguiculata
cacagttaccacatggccca

SEQ ID NO: 263 - DNA, 20 bp, Vigna_unguiculata
cctccaccaatctcaaccct

SEQ ID NO: 264 - DNA, 20 bp, Vigna_unguiculata
tcctccaccaatctcaaccc

SEQ ID NO: 265 - DNA, 20 bp, Vigna_unguiculata
acatggcccagggttgagat

SEQ ID NO: 266 - DNA, 20 bp, Vigna_unguiculata
tggcccagggttgagattgg

SEQ ID NO: 267 - DNA, 20 bp, Vigna_unguiculata
cccagggttgagattggtgg

SEQ ID NO: 268 - DNA, 20 bp, Vigna_unguiculata
tatatataccagtgtaaaga

SEQ ID NO: 269 - DNA, 20 bp, Vigna_unguiculata
tttgagatccttctttacac

SEQ ID NO: 270 - DNA, 20 bp, Vigna_unguiculata
ttttttgttcaataaataaa

SEQ ID NO: 271 - DNA, 20 bp, Vigna_unguiculata
tttcaaaacttcaatcatgt

SEQ ID NO: 272 - DNA, 20 bp, Vigna_unguiculata
catttatttcaaatgattga

SEQ ID NO: 273 - DNA, 20 bp, Vigna_unguiculata
cataatctgcaaagagaaaa

SEQ ID NO: 274 - DNA, 20 bp, Vigna_unguiculata
cactaggtcctgggacatca

SEQ ID NO: 275 - DNA, 20 bp, Vigna_unguiculata
tcactaggtcctgggacatc

SEQ ID NO: 276 - DNA, 20 bp, Vigna_unguiculata
atgacagaccctgatgtccc

SEQ ID NO: 277 - DNA, 20 bp, Vigna_unguiculata
gataagggtcactaggtcct

SEQ ID NO: 278 - DNA, 20 bp, Vigna_unguiculata
agataagggtcactaggtcc

SEQ ID NO: 279 - DNA, 20 bp, Vigna_unguiculata
tctctcagataagggtcact

SEQ ID NO: 280 - DNA, 20 bp, Vigna_unguiculata
gcagatgttctctcagataa

SEQ ID NO: 281 - DNA, 20 bp, Vigna_unguiculata
tgcagatgttctctcagata

SEQ ID NO: 282 - DNA, 20 bp, Vigna_unguiculata
atctgagagaacatctgcac

SEQ ID NO: 283 - DNA, 20 bp, Vigna_unguiculata
aaatacaaacggttaatttc

SEQ ID NO: 284 - DNA, 20 bp, Vigna_unguiculata
taaatgaagaaaaatacaaa

SEQ ID NO: 285 - DNA, 20 bp, Vigna_unguiculata
ttgtatttttcttcatttat

SEQ ID NO: 286 - DNA, 20 bp, Vigna_unguiculata
ttagaattggtttggttttc

SEQ ID NO: 287 - DNA, 20 bp, Vigna_unguiculata
ctctcagtttagaattggtt

SEQ ID NO: 288 - DNA, 20 bp, Vigna_unguiculata
tccttctctcagtttagaat

SEQ ID NO: 289 - DNA, 20 bp, Vigna_unguiculata
accaattctaaactgagaga

SEQ ID NO: 290 - DNA, 20 bp, Vigna_unguiculata
atcaattctatgcaccaaat

SEQ ID NO: 291 - DNA, 20 bp, Vigna_unguiculata
cccttcatggaaaaccaatt

SEQ ID NO: 292 - DNA, 20 bp, Vigna_unguiculata
accaaattggttttccatga

SEQ ID NO: 293 - DNA, 20 bp, Vigna_unguiculata
ccaaattggttttccatgaa

SEQ ID NO: 294 - DNA, 20 bp, Vigna_unguiculata
agaacttttataacccttca

SEQ ID NO: 295 - DNA, 20 bp, Vigna_unguiculata
acacagtaaaattaggtctt

SEQ ID NO: 296 - DNA, 20 bp, Vigna_unguiculata
tatccacacacagtaaaatt

SEQ ID NO: 297 - DNA, 20 bp, Vigna_unguiculata
agacctaattttactgtgtg

SEQ ID NO: 298 - DNA, 20 bp, Vigna_unguiculata
atcaaagcaaagaaattttc

SEQ ID NO: 299 - DNA, 20 bp, Vigna_unguiculata
ctgaaattgtgcccttttgc

SEQ ID NO: 300 - DNA, 20 bp, Vigna_unguiculata
tctgtcaccatcctgcaaaa

SEQ ID NO: 301 - DNA, 20 bp, Vigna_unguiculata
gtctgtcaccatcctgcaaa

SEQ ID NO: 302 - DNA, 20 bp, Vigna_unguiculata
aattgtgcccttttgcagga

SEQ ID NO: 303 - DNA, 20 bp, Vigna_unguiculata
aggatggtgacagacatacc

SEQ ID NO: 304 - DNA, 20 bp, Vigna_unguiculata
aatgaagcatttgttgtgcc

SEQ ID NO: 305 - DNA, 20 bp, Vigna_unguiculata
ggcacaacaaatgcttcatt

SEQ ID NO: 306 - DNA, 20 bp, Vigna_unguiculata
aaaagaacggaacaaaaaca

SEQ ID NO: 307 - DNA, 20 bp, Vigna_unguiculata
attccctaccccaaaaagaa

SEQ ID NO: 308 - DNA, 20 bp, Vigna_unguiculata
tgtttttgttccgttctttt

SEQ ID NO: 309 - DNA, 20 bp, Vigna_unguiculata
gtttttgttccgttcttttt

SEQ ID NO: 310 - DNA, 20 bp, Vigna_unguiculata
tttttgttccgttctttttg

SEQ ID NO: 311 - DNA, 20 bp, Vigna_unguiculata
tgttccgttctttttggggt

SEQ ID NO: 312 - DNA, 20 bp, Vigna_unguiculata
gttccgttctttttggggta

SEQ ID NO: 313 - DNA, 20 bp, Vigna_unguiculata
ttttggggtagggaatgtgt

SEQ ID NO: 314 - DNA, 20 bp, Vigna_unguiculata
tgtatccctatgttaggctt

SEQ ID NO: 315 - DNA, 20 bp, Vigna_unguiculata
gaaatgccaaagcctaacat

SEQ ID NO: 316 - DNA, 20 bp, Vigna_unguiculata
aaatgccaaagcctaacata

SEQ ID NO: 317 - DNA, 20 bp, Vigna_unguiculata
aacctgtgtatccctatgtt

SEQ ID NO: 318 - DNA, 20 bp, Vigna_unguiculata
agcctaacatagggatacac

SEQ ID NO: 319 - DNA, 20 bp, Vigna_unguiculata
gcctacgtttttgctggaac

SEQ ID NO: 320 - DNA, 20 bp, Vigna_unguiculata
cacactgcctacgtttttgc

SEQ ID NO: 321 - DNA, 20 bp, Vigna_unguiculata
tcctgttccagcaaaaacgt

SEQ ID NO: 322 - DNA, 20 bp, Vigna_unguiculata
aagttgtccctcgaagaagg

SEQ ID NO: 323 - DNA, 20 bp, Vigna_unguiculata
gtgttactccaccttcttcg

SEQ ID NO: 324 - DNA, 20 bp, Vigna_unguiculata
ttaaagttgtccctcgaaga

SEQ ID NO: 325 - DNA, 20 bp, Vigna_unguiculata
tgttactccaccttcttcga

SEQ ID NO: 326 - DNA, 20 bp, Vigna_unguiculata
agggagtccaaggtcgttct

SEQ ID NO: 327 - DNA, 20 bp, Vigna_unguiculata
ttttcatccgagaacgacct

SEQ ID NO: 328 - DNA, 20 bp, Vigna_unguiculata
cagcagcaacagggagtcca

SEQ ID NO: 329 - DNA, 20 bp, Vigna_unguiculata
tgaagtagacagcagcaaca

SEQ ID NO: 330 - DNA, 20 bp, Vigna_unguiculata
ttgaagtagacagcagcaac

SEQ ID NO: 331 - DNA, 20 bp, Vigna_unguiculata
ctgtctacttcaatgcacag

SEQ ID NO: 332 - DNA, 20 bp, Vigna_unguiculata
tgtctacttcaatgcacaga

SEQ ID NO: 333 - DNA, 20 bp, Vigna_unguiculata
ctagaagacgctaatatata

SEQ ID NO: 334 - DNA, 20 bp, Vigna_unguiculata
gataacgttattgagtacat

SEQ ID NO: 335 - DNA, 20 bp, Vigna_unguiculata
caataacgttatcttcacgt

SEQ ID NO: 336 - DNA, 20 bp, Vigna_unguiculata
aataacgttatcttcacgta

SEQ ID NO: 337 - DNA, 20 bp, Vigna_unguiculata
ataacgttatcttcacgtag

SEQ ID NO: 338 - DNA, 20 bp, Vigna_unguiculata
gatggtttagaagtgaaact

SEQ ID NO: 339 - DNA, 20 bp, Vigna_unguiculata
agctatttcaagcgacacga

SEQ ID NO: 340 - DNA, 1492 bp, Vigna unguiculata
catcatttctctctactctagcatgttcccttgacaattttgaagaacccaaattgcttacttaaacagtgtgttctttcccaaaatagataagcgtgtatggaatctctctttatgcttctaaaccaagccaagttcatggtttttatttcccttcataaacccttttggcgcctataaaatgccttcaaaatctcacttctcatgtgcccttcttcacaaagtttttcccttttcttagtttcattccaattcatgtctagggtcatggaaccactttctgtgggaagagtgataggagaagtggttgacattttcagcccaagtgtgagaatgaatgtgacatattccaccaaggaagttgctaatggtcatgagttaatgccttctactgttatggccaaaccacgcgtggagattggtggtgatgacatgagaactgcttataccttggtaaaatcatcactcattcaccccatctatgtgtttgatcatatgcctctattagtggtagttaactcagggtatttatgtgtcaaatggaacatttagatcatgacagacccagatgctccaagtcctagtgatccatatctaagggaacatcttcactggtttgtgtattatcaaactcaaactctccatgttttttttttcttctgtattctttgtactatatgaattccattattaacttggtgacaatttatcaggatggttacagatatccctggcaccacagatgtctcttttggttagtcactgtcttaccccacactgtgttgattcttattattattatcagtatttcttaatctaagggtaatgcttattataaaaagatgcgtgtaaaaaggacattttttgttaacaaaagggaactgttaaactggtaacaggaaaagagattatggggtatgagagtccaaaaccagtaataggaatccacagatatgtgttcatcttgttcaagcagagaggaagacaaacagtgagagctccttcttcaagagaccgtttcaacacaaggagattctcggaagagaatggccttggtctaccagttgctgcagtttacttcaatgctcaaagagaaactgctgcaaggagaaggtgatgactccagctccagaagaagaagaagaagaagaagaagaaaaaaagaaaagggtttcagttaataaagttattacttctttcaatctcaagtatctgagatttgtttctctggttttcttagggtttggcatggttgaataataaggttaggctgtgttttttatgaaatctcttcctctcttgagtttttctttttcaattttcttttggaggttggtggtgtttaaaaggtagtagatattcttgatgtactttgtacttatagcttttctaggttatgagaagttggggaccaaactatagtgttatgtttcttttaagaatctatgatttgttacaggaaagaacatagctttaccagagctgttactta

SEQ ID NO: 341 - protein, 189 aa, Vigna unguiculata
MCPSSQSFSLFLVSFQFMSRVMEPLSVGRVIGEVVDIFSPSVRMNVTYSTKEVANGHELMPSTVMAKPRVEIGGDDMRTAYTLIMTDPDAPSPSDPYLREHLHWMVTDIPGTTDVSFGKEIMGYESPKPVIGIHRYVFILFKQRGRQTVRAPSSRDRFNTRRFSEENGLGLPVAAVYFNAQRETAARRR

SEQ ID NO: 342 - DNA, 20 bp, Vigna_unguiculata
gggttcttcaaaattgtcaa

SEQ ID NO: 343 - DNA, 20 bp, Vigna_unguiculata
tgggttcttcaaaattgtca

SEQ ID NO: 344 - DNA, 20 bp, Vigna_unguiculata
actgtttaagtaagcaattt

SEQ ID NO: 345 - DNA, 20 bp, Vigna_unguiculata
cactgtttaagtaagcaatt

SEQ ID NO: 346 - DNA, 20 bp, Vigna_unguiculata
catacacgcttatctatttt

SEQ ID NO: 347 - DNA, 20 bp, Vigna_unguiculata
ccatacacgcttatctattt

SEQ ID NO: 348 - DNA, 20 bp, Vigna_unguiculata
ccaaaatagataagcgtgta

SEQ ID NO: 349 - DNA, 20 bp, Vigna_unguiculata
taaaaaccatgaacttggct

SEQ ID NO: 350 - DNA, 20 bp, Vigna_unguiculata
tctaaaccaagccaagttca

SEQ ID NO: 351 - DNA, 20 bp, Vigna_unguiculata
ggaaataaaaaccatgaact

SEQ ID NO: 352 - DNA, 20 bp, Vigna_unguiculata
gcgccaaaagggtttatgaa

SEQ ID NO: 353 - DNA, 20 bp, Vigna_unguiculata
ggcgccaaaagggtttatga

SEQ ID NO: 354 - DNA, 20 bp, Vigna_unguiculata
tttcccttcataaacccttt

SEQ ID NO: 355 - DNA, 20 bp, Vigna_unguiculata
gcattttataggcgccaaaa

SEQ ID NO: 356 - DNA, 20 bp, Vigna_unguiculata
ggcattttataggcgccaaa

SEQ ID NO: 357 - DNA, 20 bp, Vigna_unguiculata
agattttgaaggcattttat

SEQ ID NO: 358 - DNA, 20 bp, Vigna_unguiculata
catgagaagtgagattttga

SEQ ID NO: 359 - DNA, 20 bp, Vigna_unguiculata
gggaaaaactttgtgaagaa

SEQ ID NO: 360 - DNA, 20 bp, Vigna_unguiculata
agggaaaaactttgtgaaga

SEQ ID NO: 361 - DNA, 20 bp, Vigna_unguiculata
ttggaatgaaactaagaaaa

SEQ ID NO: 362 - DNA, 20 bp, Vigna_unguiculata
attggaatgaaactaagaaa

SEQ ID NO: 363 - DNA, 20 bp, Vigna_unguiculata
ccatgaccctagacatgaat

SEQ ID NO: 364 - DNA, 20 bp, Vigna_unguiculata
tttcattccaattcatgtct

SEQ ID NO: 365 - DNA, 20 bp, Vigna_unguiculata
ttcattccaattcatgtcta

SEQ ID NO: 366 - DNA, 20 bp, Vigna_unguiculata
ccaattcatgtctagggtca

SEQ ID NO: 367 - DNA, 20 bp, Vigna_unguiculata
atcactcttcccacagaaag

SEQ ID NO: 368 - DNA, 20 bp, Vigna_unguiculata
ggtcatggaaccactttctg

SEQ ID NO: 369 - DNA, 20 bp, Vigna_unguiculata
gtcatggaaccactttctgt

SEQ ID NO: 370 - DNA, 20 bp, Vigna_unguiculata
ctttctgtgggaagagtgat

SEQ ID NO: 371 - DNA, 20 bp, Vigna_unguiculata
gggaagagtgataggagaag

SEQ ID NO: 372 - DNA, 20 bp, Vigna_unguiculata
tcacattcattctcacactt

SEQ ID NO: 373 - DNA, 20 bp, Vigna_unguiculata
gtcacattcattctcacact

SEQ ID NO: 374 - DNA, 20 bp, Vigna_unguiculata
gaatgtgacatattccacca

SEQ ID NO: 375 - DNA, 20 bp, Vigna_unguiculata
accattagcaacttccttgg

SEQ ID NO: 376 - DNA, 20 bp, Vigna_unguiculata
atgaccattagcaacttcct

SEQ ID NO: 377 - DNA, 20 bp, Vigna_unguiculata
tccaccaaggaagttgctaa

SEQ ID NO: 378 - DNA, 20 bp, Vigna_unguiculata
ggtttggccataacagtaga

SEQ ID NO: 379 - DNA, 20 bp, Vigna_unguiculata
gttaatgccttctactgtta

SEQ ID NO: 380 - DNA, 20 bp, Vigna_unguiculata
accaatctccacgcgtggtt

SEQ ID NO: 381 - DNA, 20 bp, Vigna_unguiculata
tgttatggccaaaccacgcg

SEQ ID NO: 382 - DNA, 20 bp, Vigna_unguiculata
tcaccaccaatctccacgcg

SEQ ID NO: 383 - DNA, 20 bp, Vigna_unguiculata
gccaaaccacgcgtggagat

SEQ ID NO: 384 - DNA, 20 bp, Vigna_unguiculata
aaaccacgcgtggagattgg

SEQ ID NO: 385 - DNA, 20 bp, Vigna_unguiculata
catgagaactgcttatacct

SEQ ID NO: 386 - DNA, 20 bp, Vigna_unguiculata
aatgagtgatgattttacca

SEQ ID NO: 387 - DNA, 20 bp, Vigna_unguiculata
tatgatcaaacacatagatg

SEQ ID NO: 388 - DNA, 20 bp, Vigna_unguiculata
atatgatcaaacacatagat

SEQ ID NO: 389 - DNA, 20 bp, Vigna_unguiculata
catatgatcaaacacataga

SEQ ID NO: 390 - DNA, 20 bp, Vigna_unguiculata
gagttaactaccactaatag

SEQ ID NO: 391 - DNA, 20 bp, Vigna_unguiculata
tgatcatatgcctctattag

SEQ ID NO: 392 - DNA, 20 bp, Vigna_unguiculata
ctattagtggtagttaactc

SEQ ID NO: 393 - DNA, 20 bp, Vigna_unguiculata
tattagtggtagttaactca

SEQ ID NO: 394 - DNA, 20 bp, Vigna_unguiculata
cagggtatttatgtgtcaaa

SEQ ID NO: 395 - DNA, 20 bp, Vigna_unguiculata
cactaggacttggagcatct

SEQ ID NO: 396 - DNA, 20 bp, Vigna_unguiculata
tcactaggacttggagcatc

SEQ ID NO: 397 - DNA, 20 bp, Vigna_unguiculata
agatatggatcactaggact

SEQ ID NO: 398 - DNA, 20 bp, Vigna_unguiculata
tcccttagatatggatcact

SEQ ID NO: 399 - DNA, 20 bp, Vigna_unguiculata
gtcctagtgatccatatcta

SEQ ID NO: 400 - DNA, 20 bp, Vigna_unguiculata
tgaagatgttcccttagata

SEQ ID NO: 401 - DNA, 20 bp, Vigna_unguiculata
tcctagtgatccatatctaa

SEQ ID NO: 402 - DNA, 20 bp, Vigna_unguiculata
atctaagggaacatcttcac

SEQ ID NO: 403 - DNA, 20 bp, Vigna_unguiculata
tacagaagaaaaaaaaaaca

SEQ ID NO: 404 - DNA, 20 bp, Vigna_unguiculata
aattgtcaccaagttaataa

SEQ ID NO: 405 - DNA, 20 bp, Vigna_unguiculata
tatgaattccattattaact

SEQ ID NO: 406 - DNA, 20 bp, Vigna_unguiculata
taacttggtgacaatttatc

SEQ ID NO: 407 - DNA, 20 bp, Vigna_unguiculata
ttggtgacaatttatcagga

SEQ ID NO: 408 - DNA, 20 bp, Vigna_unguiculata
aggatggttacagatatccc

SEQ ID NO: 409 - DNA, 20 bp, Vigna_unguiculata
aagagacatctgtggtgcca

SEQ ID NO: 410 - DNA, 20 bp, Vigna_unguiculata
aaagagacatctgtggtgcc

SEQ ID NO: 411 - DNA, 20 bp, Vigna_unguiculata
ctaaccaaaagagacatctg

SEQ ID NO: 412 - DNA, 20 bp, Vigna_unguiculata
ggcaccacagatgtctcttt

SEQ ID NO: 413 - DNA, 20 bp, Vigna_unguiculata
ataagaatcaacacagtgtg

SEQ ID NO: 414 - DNA, 20 bp, Vigna_unguiculata
aataagaatcaacacagtgt

SEQ ID NO: 415 - DNA, 20 bp, Vigna_unguiculata
taataagaatcaacacagtg

SEQ ID NO: 416 - DNA, 20 bp, Vigna_unguiculata
tatcagtatttcttaatcta

SEQ ID NO: 417 - DNA, 20 bp, Vigna_unguiculata
atcagtatttcttaatctaa

SEQ ID NO: 418 - DNA, 20 bp, Vigna_unguiculata
ataaaaagatgcgtgtaaaa

SEQ ID NO: 419 - DNA, 20 bp, Vigna_unguiculata
ggacattttttgttaacaaa

SEQ ID NO: 420 - DNA, 20 bp, Vigna_unguiculata
gacattttttgttaacaaaa

SEQ ID NO: 421 - DNA, 20 bp, Vigna_unguiculata
acaaaagggaactgttaaac

SEQ ID NO: 422 - DNA, 20 bp, Vigna_unguiculata
ggaactgttaaactggtaac

SEQ ID NO: 423 - DNA, 20 bp, Vigna_unguiculata
ggtaacaggaaaagagatta

SEQ ID NO: 424 - DNA, 20 bp, Vigna_unguiculata
gtaacaggaaaagagattat

SEQ ID NO: 425 - DNA, 20 bp, Vigna_unguiculata
taacaggaaaagagattatg

SEQ ID NO: 426 - DNA, 20 bp, Vigna_unguiculata
tggattcctattactggttt

SEQ ID NO: 427 - DNA, 20 bp, Vigna_unguiculata
gagagtccaaaaccagtaat

SEQ ID NO: 428 - DNA, 20 bp, Vigna_unguiculata
tatctgtggattcctattac

SEQ ID NO: 429 - DNA, 20 bp, Vigna_unguiculata
acaagatgaacacatatctg

SEQ ID NO: 430 - DNA, 20 bp, Vigna_unguiculata
ttcatcttgttcaagcagag

SEQ ID NO: 431 - DNA, 20 bp, Vigna_unguiculata
ttgaaacggtctcttgaaga

SEQ ID NO: 432 - DNA, 20 bp, Vigna_unguiculata
agaatctccttgtgttgaaa

SEQ ID NO: 433 - DNA, 20 bp, Vigna_unguiculata
caagagaccgtttcaacaca

SEQ ID NO: 434 - DNA, 20 bp, Vigna_unguiculata
tttcaacacaaggagattct

SEQ ID NO: 435 - DNA, 20 bp, Vigna_unguiculata
aggagattctcggaagagaa

SEQ ID NO: 436 - DNA, 20 bp, Vigna_unguiculata
ttctcggaagagaatggcct

SEQ ID NO: 437 - DNA, 20 bp, Vigna_unguiculata
ctgcagcaactggtagacca

SEQ ID NO: 438 - DNA, 20 bp, Vigna_unguiculata
ttgaagtaaactgcagcaac

SEQ ID NO: 439 - DNA, 20 bp, Vigna_unguiculata
ctcaaagagaaactgctgca

SEQ ID NO: 440 - DNA, 20 bp, Vigna_unguiculata
gagaaactgctgcaaggaga

SEQ ID NO: 441 - DNA, 20 bp, Vigna_unguiculata
tcttcttcttcttctggagc

SEQ ID NO: 442 - DNA, 20 bp, Vigna_unguiculata
tcttcttcttcttcttcttc

SEQ ID NO: 443 - DNA, 20 bp, Vigna_unguiculata
aagaagaagaaaaaaagaaa

SEQ ID NO: 444 - DNA, 20 bp, Vigna_unguiculata
agaagaagaaaaaaagaaaa

SEQ ID NO: 445 - DNA, 20 bp, Vigna_unguiculata
gtatctgagatttgtttctc

SEQ ID NO: 446 - DNA, 20 bp, Vigna_unguiculata
tttgtttctctggttttctt

SEQ ID NO: 447 - DNA, 20 bp, Vigna_unguiculata
ttgtttctctggttttctta

SEQ ID NO: 448 - DNA, 20 bp, Vigna_unguiculata
tctctggttttcttagggtt

SEQ ID NO: 449 - DNA, 20 bp, Vigna_unguiculata
ggttttcttagggtttggca

SEQ ID NO: 450 - DNA, 20 bp, Vigna_unguiculata
tttggcatggttgaataata

SEQ ID NO: 451 - DNA, 20 bp, Vigna_unguiculata
catggttgaataataaggtt

SEQ ID NO: 452 - DNA, 20 bp, Vigna_unguiculata
gaaaaagaaaaactcaagag

SEQ ID NO: 453 - DNA, 20 bp, Vigna_unguiculata
tttctttttcaattttcttt

SEQ ID NO: 454 - DNA, 20 bp, Vigna_unguiculata
ctttttcaattttcttttgg

SEQ ID NO: 455 - DNA, 20 bp, Vigna_unguiculata
ttcaattttcttttggaggt

SEQ ID NO: 456 - DNA, 20 bp, Vigna_unguiculata
aattttcttttggaggttgg

SEQ ID NO: 457 - DNA, 20 bp, Vigna_unguiculata
ggaggttggtggtgtttaaa

SEQ ID NO: 458 - DNA, 20 bp, Vigna_unguiculata
tttgtacttatagcttttct

SEQ ID NO: 459 - DNA, 20 bp, Vigna_unguiculata
ttttctaggttatgagaagt

SEQ ID NO: 460 - DNA, 20 bp, Vigna_unguiculata
tttctaggttatgagaagtt

SEQ ID NO: 461 - DNA, 20 bp, Vigna_unguiculata
ttctaggttatgagaagttg

SEQ ID NO: 462 - DNA, 20 bp, Vigna_unguiculata
agaaacataacactatagtt

SEQ ID NO: 463 - DNA, 20 bp, Vigna_unguiculata
aagaatctatgatttgttac

SEQ ID NO: 464 - DNA, 2721 bp, Vigna unguiculata
accattgtgggtgtagtacaatgtgtgtgaaccaaaggcatattgtgtttagcagtgagttagagttagccatgcctactactagcagaaatcctcttgtggttgggcgtgttataggggaggtaatagaacactttgaaagttccattccttttagggtctcctatggcaatagagaggtcaacaatggttgtgagcttaaaccttcccaagttgtgaacaaacccagagtgagtgttggtggagatgatctcaggaacttctatactctggtaattcattgcattactttcttcttatagggtttggttacacacaacctaaaagtgtttaccaaaacctctttactaaaagtaccaaaccctttttcattaaggttttcagaaacacttccagatatttatatatatcacttaaaatctgttcaatttttttctgttaaaggtgttagtagatcctgatgctcccagccctagtaaccctaatttcagggagtaccttcattggtgagtaaacaagacatatcatgtttttctagcatgacattagtaacatatcattgtactaacattctttttctcgatggattttgtgtaggttggtgactgatattcccgcaactaccggggctagtttcggtgtgtaatagttaacttgaaaaaactaaaatataaaacctctattaacaattaattagacaattttaaaaagaataagaaaaaattgtggttaaaaatatgcaagttctgttttactgactcatcaaatgtaatgtctaatgtgaatttaacccactctaacgtattcctaaaatattcatatgcaaaactataatgtgcttaatttgataaatttatggattcctgagttttaagttttgaattacgtgggattttccagatttaacttataattcctagaaagttaaattgatattaagaattaggattgtctttattagtctaacttattatctctaaccaaaaaatataatctgtcagtataaatattatgaatgcattaagattatttatattaatagtttatatattgataattcatattaactatctgtgctaatggaattgtcagttagcatgatttatcatgcgtttagactttgtgtgttagtcaaagattatagtaaaatcgactgacccaatgccatagtagtataaaataactttttttatactgaaatgagttattttcattttaactcatctggtacttaatgtataattattacttctaatcatgcctaaaagttagcgtacttacaaatacaagaatcgtctggattgtctagattcttgtaagccttttattttttttcttttgaaccttttttctgtgttaaagtctctttatactttttcaatatatatatatatatatatatatatatatataactaaagctttagtttagaagataacagtccaagcaaacatatataaaacgaatagaaaatgtaagtgcatgtaaaattagttacttatgtgatatctttaaaatctagttcacgctagttctatcataggtttaattcatttatttgatatgtttttaattatcaaaaggtataaaaaataatccaacaattctacaattcatgtcagtgacgttatcaattattatttcatttcagcaagaatacaaaaataaaaatagtttgagaatattttgtgatttcgttgttttatgtttcttgcagctacatcactgtgtttgttgatacaaaattttaacaaaaatagttataacatgaatgtataaaagccaataaactcaacattttgtatctgctatagcattgcgatcttctagtttgtcgacaagtatgcataactataattagattttgggacctatatatcacatcatgcggaacattgtgtcgtttagaatttaaagggcttgtcgtgcaggaaagaaaaaggagtataaaagtattggaatcatttatgtggaccatgtttctttatatatacttgaagtacaaaaatggtgtgtgtgatgcacaatatcatcacatggaagaaattgaaattgattccacgaggcatcaaattctaaattgtgtatgatgagacaaatgacaccagctaccaaaaggataaaaaggaaatatcatgggaaatatctaaggcccaaaaatactgaattatgataatctaatatcaatatcaatatcaatgaactggtataagagatgtgtatgagtggttcaattataacatgctaactggggtttttgcatgatttgatcgtaggtaacgaggttgtaagttatgaaagtccacgaccaacgatggggattcatcgtttggtgtttgtgttgttccgtcaacagtatcgacagagggtgtatgctcctggatggcgacagaatttcaacaccagagaatttgctgaactttacaatcttggtttgccagttgctgcagtgttcttcaactgtcagagggaaactggttctggtggtaggacattttgaaagttactttgtaccaaaaccgcacatgttttagttaatgaaagcacctaaaataaagaaacctacgtaatatatatgtaacaacaataataacacacatcgagtaataggaatatgtaaccaaaaatacatgtactactttatgtttttatatatatataaaaatgaaatttgactagttgatcaaatataatgaaggcactaagaataacagtgaggca

SEQ ID NO: 465 - protein, 193 aa, Vigna unguiculata
MCVNQRHIVFSSELELAMPTTSRNPLVVGRVIGEVIEHFESSIPFRVSYGNREVNNGCELKPSQVVNKPRVSVGGDDLRNFYTLVLVDPDAPSPSNPNFREYLHWLVTDIPATTGASFGNEVVSYESPRPTMGIHRLVFVLFRQQYRQRVYAPGWRQNFNTREFAELYNLGLPVAAVFFNCQRETGSGGRTFV

SEQ ID NO: 466 - DNA, 20 bp, Vigna_unguiculata
acattgtactacacccacaa

SEQ ID NO: 467 - DNA, 20 bp, Vigna_unguiculata
gtacaatgtgtgtgaaccaa

SEQ ID NO: 468 - DNA, 20 bp, Vigna_unguiculata
tgctaaacacaatatgcctt

SEQ ID NO: 469 - DNA, 20 bp, Vigna_unguiculata
atttctgctagtagtaggca

SEQ ID NO: 470 - DNA, 20 bp, Vigna_unguiculata
agaggatttctgctagtagt

SEQ ID NO: 471 - DNA, 20 bp, Vigna_unguiculata
tactagcagaaatcctcttg

SEQ ID NO: 472 - DNA, 20 bp, Vigna_unguiculata
ataacacgcccaaccacaag

SEQ ID NO: 473 - DNA, 20 bp, Vigna_unguiculata
agcagaaatcctcttgtggt

SEQ ID NO: 474 - DNA, 20 bp, Vigna_unguiculata
gcagaaatcctcttgtggtt

SEQ ID NO: 475 - DNA, 20 bp, Vigna_unguiculata
cttgtggttgggcgtgttat

SEQ ID NO: 476 - DNA, 20 bp, Vigna_unguiculata
ttgtggttgggcgtgttata

SEQ ID NO: 477 - DNA, 20 bp, Vigna_unguiculata
tgtggttgggcgtgttatag

SEQ ID NO: 478 - DNA, 20 bp, Vigna_unguiculata
ggttgggcgtgttatagggg

SEQ ID NO: 479 - DNA, 20 bp, Vigna_unguiculata
ataggagaccctaaaaggaa

SEQ ID NO: 480 - DNA, 20 bp, Vigna_unguiculata
ttgaaagttccattcctttt

SEQ ID NO: 481 - DNA, 20 bp, Vigna_unguiculata
tgaaagttccattcctttta

SEQ ID NO: 482 - DNA, 20 bp, Vigna_unguiculata
ttgccataggagaccctaaa

SEQ ID NO: 483 - DNA, 20 bp, Vigna_unguiculata
attccttttagggtctccta

SEQ ID NO: 484 - DNA, 20 bp, Vigna_unguiculata
gttgacctctctattgccat

SEQ ID NO: 485 - DNA, 20 bp, Vigna_unguiculata
ggtctcctatggcaatagag

SEQ ID NO: 486 - DNA, 20 bp, Vigna_unguiculata
ggcaatagagaggtcaacaa

SEQ ID NO: 487 - DNA, 20 bp, Vigna_unguiculata
ggtttgttcacaacttggga

SEQ ID NO: 488 - DNA, 20 bp, Vigna_unguiculata
tctgggtttgttcacaactt

SEQ ID NO: 489 - DNA, 20 bp, Vigna_unguiculata
ctctgggtttgttcacaact

SEQ ID NO: 490 - DNA, 20 bp, Vigna_unguiculata
tctccaccaacactcactct

SEQ ID NO: 491 - DNA, 20 bp, Vigna_unguiculata
atctccaccaacactcactc

SEQ ID NO: 492 - DNA, 20 bp, Vigna_unguiculata
aacaaacccagagtgagtgt

SEQ ID NO: 493 - DNA, 20 bp, Vigna_unguiculata
aaacccagagtgagtgttgg

SEQ ID NO: 494 - DNA, 20 bp, Vigna_unguiculata
gtgttggtggagatgatctc

SEQ ID NO: 495 - DNA, 20 bp, Vigna_unguiculata
tctcaggaacttctatactc

SEQ ID NO: 496 - DNA, 20 bp, Vigna_unguiculata
ttgcattactttcttcttat

SEQ ID NO: 497 - DNA, 20 bp, Vigna_unguiculata
tgcattactttcttcttata

SEQ ID NO: 498 - DNA, 20 bp, Vigna_unguiculata
tactttcttcttatagggtt

SEQ ID NO: 499 - DNA, 20 bp, Vigna_unguiculata
aggttttggtaaacactttt

SEQ ID NO: 500 - DNA, 20 bp, Vigna_unguiculata
tacttttagtaaagaggttt

SEQ ID NO: 501 - DNA, 20 bp, Vigna_unguiculata
gtttggtacttttagtaaag

SEQ ID NO: 502 - DNA, 20 bp, Vigna_unguiculata
aaccttaatgaaaaagggtt

SEQ ID NO: 503 - DNA, 20 bp, Vigna_unguiculata
ctgaaaaccttaatgaaaaa

SEQ ID NO: 504 - DNA, 20 bp, Vigna_unguiculata
tctgaaaaccttaatgaaaa

SEQ ID NO: 505 - DNA, 20 bp, Vigna_unguiculata
taccaaaccctttttcatta

SEQ ID NO: 506 - DNA, 20 bp, Vigna_unguiculata
aagtgatatatataaatatc

SEQ ID NO: 507 - DNA, 20 bp, Vigna_unguiculata
gttcaatttttttctgttaa

SEQ ID NO: 508 - DNA, 20 bp, Vigna_unguiculata
ttactagggctgggagcatc

SEQ ID NO: 509 - DNA, 20 bp, Vigna_unguiculata
aaattagggttactagggct

SEQ ID NO: 510 - DNA, 20 bp, Vigna_unguiculata
gaaattagggttactagggc

SEQ ID NO: 511 - DNA, 20 bp, Vigna_unguiculata
ccctgaaattagggttacta

SEQ ID NO: 512 - DNA, 20 bp, Vigna_unguiculata
tccctgaaattagggttact

SEQ ID NO: 513 - DNA, 20 bp, Vigna_unguiculata
gaaggtactccctgaaatta

SEQ ID NO: 514 - DNA, 20 bp, Vigna_unguiculata
gccctagtaaccctaatttc

SEQ ID NO: 515 - DNA, 20 bp, Vigna_unguiculata
tgaaggtactccctgaaatt

SEQ ID NO: 516 - DNA, 20 bp, Vigna_unguiculata
ccctagtaaccctaatttca

SEQ ID NO: 517 - DNA, 20 bp, Vigna_unguiculata
atttcagggagtaccttcat

SEQ ID NO: 518 - DNA, 20 bp, Vigna_unguiculata
tcttgtttactcaccaatga

SEQ ID NO: 519 - DNA, 20 bp, Vigna_unguiculata
actaacattctttttctcga

SEQ ID NO: 520 - DNA, 20 bp, Vigna_unguiculata
tttctcgatggattttgtgt

SEQ ID NO: 521 - DNA, 20 bp, Vigna_unguiculata
tcgatggattttgtgtaggt

SEQ ID NO: 522 - DNA, 20 bp, Vigna_unguiculata
aaactagccccggtagttgc

SEQ ID NO: 523 - DNA, 20 bp, Vigna_unguiculata
gaaactagccccggtagttg

SEQ ID NO: 524 - DNA, 20 bp, Vigna_unguiculata
actgatattcccgcaactac

SEQ ID NO: 525 - DNA, 20 bp, Vigna_unguiculata
ctgatattcccgcaactacc

SEQ ID NO: 526 - DNA, 20 bp, Vigna_unguiculata
tgatattcccgcaactaccg

SEQ ID NO: 527 - DNA, 20 bp, Vigna_unguiculata
ttacacaccgaaactagccc

SEQ ID NO: 528 - DNA, 20 bp, Vigna_unguiculata
gcaactaccggggctagttt

SEQ ID NO: 529 - DNA, 20 bp, Vigna_unguiculata
gtctaattaattgttaatag

SEQ ID NO: 530 - DNA, 20 bp, Vigna_unguiculata
aaaagaataagaaaaaattg

SEQ ID NO: 531 - DNA, 20 bp, Vigna_unguiculata
ttttaggaatacgttagagt

SEQ ID NO: 532 - DNA, 20 bp, Vigna_unguiculata
attttaggaatacgttagag

SEQ ID NO: 533 - DNA, 20 bp, Vigna_unguiculata
gttttgcatatgaatatttt

SEQ ID NO: 534 - DNA, 20 bp, Vigna_unguiculata
tgcttaatttgataaattta

SEQ ID NO: 535 - DNA, 20 bp, Vigna_unguiculata
taattcaaaacttaaaactc

SEQ ID NO: 536 - DNA, 20 bp, Vigna_unguiculata
gttttaagttttgaattacg

SEQ ID NO: 537 - DNA, 20 bp, Vigna_unguiculata
ttttaagttttgaattacgt

SEQ ID NO: 538 - DNA, 20 bp, Vigna_unguiculata
taggaattataagttaaatc

SEQ ID NO: 539 - DNA, 20 bp, Vigna_unguiculata
taatatcaatttaactttct

SEQ ID NO: 540 - DNA, 20 bp, Vigna_unguiculata
ttaaattgatattaagaatt

SEQ ID NO: 541 - DNA, 20 bp, Vigna_unguiculata
tactgacagattatattttt

SEQ ID NO: 542 - DNA, 20 bp, Vigna_unguiculata
atattaactatctgtgctaa

SEQ ID NO: 543 - DNA, 20 bp, Vigna_unguiculata
ttttatactactatggcatt

SEQ ID NO: 544 - DNA, 20 bp, Vigna_unguiculata
attttatactactatggcat

SEQ ID NO: 545 - DNA, 20 bp, Vigna_unguiculata
aaagttattttatactacta

SEQ ID NO: 546 - DNA, 20 bp, Vigna_unguiculata
tattttcattttaactcatc

SEQ ID NO: 547 - DNA, 20 bp, Vigna_unguiculata
ttgtaagtacgctaactttt

SEQ ID NO: 548 - DNA, 20 bp, Vigna_unguiculata
ttacaaatacaagaatcgtc

SEQ ID NO: 549 - DNA, 20 bp, Vigna_unguiculata
ttcaaaagaaaaaaaataaa

SEQ ID NO: 550 - DNA, 20 bp, Vigna_unguiculata
gagactttaacacagaaaaa

SEQ ID NO: 551 - DNA, 20 bp, Vigna_unguiculata
tcgttttatatatgtttgct

SEQ ID NO: 552 - DNA, 20 bp, Vigna_unguiculata
gttcacgctagttctatcat

SEQ ID NO: 553 - DNA, 20 bp, Vigna_unguiculata
atatgtttttaattatcaaa

SEQ ID NO: 554 - DNA, 20 bp, Vigna_unguiculata
gacatgaattgtagaattgt

SEQ ID NO: 555 - DNA, 20 bp, Vigna_unguiculata
atacaaaatgttgagtttat

SEQ ID NO: 556 - DNA, 20 bp, Vigna_unguiculata
gcataactataattagattt

SEQ ID NO: 557 - DNA, 20 bp, Vigna_unguiculata
cataactataattagatttt

SEQ ID NO: 558 - DNA, 20 bp, Vigna_unguiculata
ttccgcatgatgtgatatat

SEQ ID NO: 559 - DNA, 20 bp, Vigna_unguiculata
gacctatatatcacatcatg

SEQ ID NO: 560 - DNA, 20 bp, Vigna_unguiculata
ttgtgtcgtttagaatttaa

SEQ ID NO: 561 - DNA, 20 bp, Vigna_unguiculata
tgtgtcgtttagaatttaaa

SEQ ID NO: 562 - DNA, 20 bp, Vigna_unguiculata
atttaaagggcttgtcgtgc

SEQ ID NO: 563 - DNA, 20 bp, Vigna_unguiculata
ttgtcgtgcaggaaagaaaa

SEQ ID NO: 564 - DNA, 20 bp, Vigna_unguiculata
aaaaaggagtataaaagtat

SEQ ID NO: 565 - DNA, 20 bp, Vigna_unguiculata
aagtattggaatcatttatg

SEQ ID NO: 566 - DNA, 20 bp, Vigna_unguiculata
caagtatatataaagaaaca

SEQ ID NO: 567 - DNA, 20 bp, Vigna_unguiculata
atatacttgaagtacaaaaa

SEQ ID NO: 568 - DNA, 20 bp, Vigna_unguiculata
tgatgcacaatatcatcaca

SEQ ID NO: 569 - DNA, 20 bp, Vigna_unguiculata
aaattgaaattgattccacg

SEQ ID NO: 570 - DNA, 20 bp, Vigna_unguiculata
atttagaatttgatgcctcg

SEQ ID NO: 571 - DNA, 20 bp, Vigna_unguiculata
ctttttatccttttggtagc

SEQ ID NO: 572 - DNA, 20 bp, Vigna_unguiculata
aaatgacaccagctaccaaa

SEQ ID NO: 573 - DNA, 20 bp, Vigna_unguiculata
atatttcctttttatccttt

SEQ ID NO: 574 - DNA, 20 bp, Vigna_unguiculata
cagctaccaaaaggataaaa

SEQ ID NO: 575 - DNA, 20 bp, Vigna_unguiculata
aggataaaaaggaaatatca

SEQ ID NO: 576 - DNA, 20 bp, Vigna_unguiculata
ggataaaaaggaaatatcat

SEQ ID NO: 577 - DNA, 20 bp, Vigna_unguiculata
aatatcatgggaaatatcta

SEQ ID NO: 578 - DNA, 20 bp, Vigna_unguiculata
tatcataattcagtattttt

SEQ ID NO: 579 - DNA, 20 bp, Vigna_unguiculata
ttatcataattcagtatttt

SEQ ID NO: 580 - DNA, 20 bp, Vigna_unguiculata
caatatcaatatcaatgaac

SEQ ID NO: 581 - DNA, 20 bp, Vigna_unguiculata
gtataagagatgtgtatgag

SEQ ID NO: 582 - DNA, 20 bp, Vigna_unguiculata
ttcaattataacatgctaac

SEQ ID NO: 583 - DNA, 20 bp, Vigna_unguiculata
tcaattataacatgctaact

SEQ ID NO: 584 - DNA, 20 bp, Vigna_unguiculata
caattataacatgctaactg

SEQ ID NO: 585 - DNA, 20 bp, Vigna_unguiculata
tttttgcatgatttgatcgt

SEQ ID NO: 586 - DNA, 20 bp, Vigna_unguiculata
tgatttgatcgtaggtaacg

SEQ ID NO: 587 - DNA, 20 bp, Vigna_unguiculata
tgaatccccatcgttggtcg

SEQ ID NO: 588 - DNA, 20 bp, Vigna_unguiculata
tgaaagtccacgaccaacga

SEQ ID NO: 589 - DNA, 20 bp, Vigna_unguiculata
gaaagtccacgaccaacgat

SEQ ID NO: 590 - DNA, 20 bp, Vigna_unguiculata
aaagtccacgaccaacgatg

SEQ ID NO: 591 - DNA, 20 bp, Vigna_unguiculata
aaacgatgaatccccatcgt

SEQ ID NO: 592 - DNA, 20 bp, Vigna_unguiculata
aacgatggggattcatcgtt

SEQ ID NO: 593 - DNA, 20 bp, Vigna_unguiculata
ccctctgtcgatactgttga

SEQ ID NO: 594 - DNA, 20 bp, Vigna_unguiculata
tccgtcaacagtatcgacag

SEQ ID NO: 595 - DNA, 20 bp, Vigna_unguiculata
ccgtcaacagtatcgacaga

SEQ ID NO: 596 - DNA, 20 bp, Vigna_unguiculata
cgacagagggtgtatgctcc

SEQ ID NO: 597 - DNA, 20 bp, Vigna_unguiculata
agagggtgtatgctcctgga

SEQ ID NO: 598 - DNA, 20 bp, Vigna_unguiculata
ttgaaattctgtcgccatcc

SEQ ID NO: 599 - DNA, 20 bp, Vigna_unguiculata
gtaaagttcagcaaattctc

SEQ ID NO: 600 - DNA, 20 bp, Vigna_unguiculata
tttgctgaactttacaatct

SEQ ID NO: 601 - DNA, 20 bp, Vigna_unguiculata
ttgaagaacactgcagcaac

SEQ ID NO: 602 - DNA, 20 bp, Vigna_unguiculata
cagtgttcttcaactgtcag

SEQ ID NO: 603 - DNA, 20 bp, Vigna_unguiculata
agtgttcttcaactgtcaga

SEQ ID NO: 604 - DNA, 20 bp, Vigna_unguiculata
ttcaactgtcagagggaaac

SEQ ID NO: 605 - DNA, 20 bp, Vigna_unguiculata
tgtcagagggaaactggttc

SEQ ID NO: 606 - DNA, 20 bp, Vigna_unguiculata
cagagggaaactggttctgg

SEQ ID NO: 607 - DNA, 20 bp, Vigna_unguiculata
gggaaactggttctggtggt

SEQ ID NO: 608 - DNA, 20 bp, Vigna_unguiculata
aactaaaacatgtgcggttt

SEQ ID NO: 609 - DNA, 20 bp, Vigna_unguiculata
ttcattaactaaaacatgtg

SEQ ID NO: 610 - DNA, 20 bp, Vigna_unguiculata
tacgtaggtttctttatttt

SEQ ID NO: 611 - DNA, 20 bp, Vigna_unguiculata
ttgttacatatatattacgt

SEQ ID NO: 612 - DNA, 20 bp, Vigna_unguiculata
aataacacacatcgagtaat

SEQ ID NO: 613 - DNA, 20 bp, Vigna_unguiculata
taaagtagtacatgtatttt

SEQ ID NO: 614 - DNA, 20 bp, Vigna_unguiculata
tagttgatcaaatataatga

SEQ ID NO: 615 - DNA, 20 bp, Vigna_unguiculata
aggcactaagaataacagtg

SEQ ID NO: 616 - DNA, 4550 bp, Vigna unguiculata
agaaaagcaaataagattgaaataatgcgtggaacaacgaaccctcttgttgttggacgtgtaataggagatgtggtggaaccatttgcatgttcgattcctctgagagttgtgtacaaccataacaaagaagtgatcaacagtggtgaactcaaaccctcccaaataatcatccctccaagagttgaggttggtggagatgatctcaggaccctttacactttggtacaaccttttacttttcttaaacctaaaatcaaacttctcatcttcttcattaagctaaatcatgctcattcatgcatgtatactaatcttgcaggtcatggtggaccctgatgcacccagcccaagtaacccaactatgagggaatatcttcactggtaattattaacttctcaatacaattcttatctcaatcacttgcttattttttaagctatataaattagggtttaagatttaggatttagatattagatatgaaaaacttatctgattaataaatagatataaatagatagatggtgttggaatgtaaagagatattgaattttctgcataattgtatcctctttgtcttggtttttgaatccattgaacaccacaaacatcgtcaccagcactatgtgagtgcttgtgttttcaggtttgttatataccgaggagcttcttttatgcagctcaaactttcttcctaaacttatgaaaaatatatgattctcatttttctgattcttttattttctgacatggtttagtgtgagttattgattaatatattattattttttttctttaaaaaaatcaatgatccacactaagtcacatcataaagtaaatgatgagagtcaaaaaataggaatcacaatgtaattttcctaaatatatatgtataaaagtcaaagaaaaactaactggacaataatattttgacaactaaattttaataattttttttacaacctgaaatgatattttttaatttgttttagaatacataaaaatgagaaaatagaaaaataaaaagtcacttaaaatatgtcactaatcataaaagaagattgtcgaaatttagttgttaaaaaatcattaaattatttttaacatttttcattgtacaatttgaaaatacatttccaattacatgatttaaaaataaaaataaaaataaaaataaatgtataattaaaaaacctaaaaacattttctaactttttggaatatattttatagttgaaatttgttttttcaacccatggatcatagggtgagagagagaaaaaaaatagaatggataaagagaggggtgcgtcatgtcatggcccaatgggccttcgatgggtattcgtttcggtcgtcataatttttttcttgtttattttataatgcaaccaatttaactacataacttcttaaaatatcgacaaaatgattgaacaaacgtaaaataactgatataagttacagatataacttgccaatttacctttttagtttaattttgatcatttgtaatatggtgtaatttatgttatgagtgaaaaatataacaactattattttttttattcaacacactttttatttattttacactattaaaatctttttgtatagggaagttatttttctaaaagaggagtacttacaagttttaagaaaagaggactaacaagttaacaacacattttctaacataactttttcttgatacacacttcattgatcagaacttgttgaaaactaaatggataggattcagagttagtaaccgataatcaatttataaaaatattaattatcatactttatattttattaatgagaaagacttacatcaacccacacgtagactttttcttttgtaatatgaaagctatatatatatatatatatatatatatatatatatatatatatatatatatatatatatatatttgtgatttttaataaacttcaataaataataaaatattgtaataacagtttacttcaacaaaaaaaaaaaaaaacctatttttctactttcactccaatataatagttttaaacttttacatccataacattttcttttctttcaataaaacgtatgttgaattactgtgttaatatgatagaaaaaaaaaatcattgaatcaaaattaaaaatgataaataattaaaagaaaaatgtaattatgaaaacttattagtttttgtaattatgttttttttagttatatataacttttaatctcgtttcgtcaattcttattatttgtttttttatttttattttataatgactaatgattattacaatgattactttaactataatcattaactagaactagaatagtttattttaaaagtaagattgaatttatgtcataaaagattatctttacaatcaaaattacttaataattgtgtgtaagactgtttgtctggatcgaaaaatccgatgattcaattatactagtgtacatataagcgttgtcacgcctgtatgtacgcattttttaaatatgcgtgatattatattcataggtgatgataatgtattgtttttaagttaatatatcagaaaagtttagagaataatggttgataaacagagaaaaaaagaaaaagcaaagatagatgatttaagaaaaaacttgtaaaaataatggtcaattttaaaaaatgtaataaataattatattttaaataataaaattgatattttaaaatgtgaacacaaatagaatcttttttatatatttatagattatgttaacatttaattttttttatcgagtaaaatccaattcagtccaaactaattcattttcatacgatttcatgtaataagtgtagtatgagtgagaaacccaacttagaatcatttttatacatgtactgtggacaaattatgttatattattagtttttgtcgagaattaatttgaacaaagttttcattgatgtattgatttaattgaagcttaatcaatcttttgtaaagattatattatcttttacgtagattgatacttaataattacacctattaataaaaaaaatcaaaattttgaccaattgatagaacccaatctaatttactcaacaatgggctgagttggattcagattagtgtaaaaaatgcagaaaatgtcaacccaacctgatattgggtaatgaatttattcagatctatcaagttcagcccgctaacatctctgatatgtctatattttaaaaaataatttacattatatatatatatatatatatatatatatatatatatatatatatatatatatatatatatatatatatatatatatatattattgatctatgtctacggatcaagttaacattaaaagaattattattcacctttttttttctttctaactattgtaattaataagtataatttttaaatggtaaaagaaaataagatcaaaattaactcttttgaacttacaacatcactgagccaataatcttttttagtttttaaggattatgttaagaagctaccctttaagtatattttaaaatacataataaaaatttgctaacatacttctaaaatacgtaagatataacttgctaaagttcctttgtttaaaaaaatgtatgtatgctatcaattctttgtgtttgttagtatacatcaattttttaaaataattagtaagttataattacgattatgttagaattagtctaaaacgctatttctttttcttcgatggatctttaggatggtaatcaacattccagcaacaacagggacaaactttggtacgtacctgaactcttttatttttatttttattactactttcttctattgtttttttattattaaccatcatatatatgttgggttatatattcttttacatatatggttataagagattgtaatttcatatggatatatatgacattagtcttacacatataaaatatttgatatcacttttacaccaaaatcttaaaacaatagtgttatgggtctttattcttatatagtgtttaactttgtctattatatccaatgttagactttgactcacacttgagctatttccaacaatatatattaatttgacaggagaagagattgtgagttatgaaagtccaagaccaacatcagggattcatcgtttagtatttgtgttgtttaaacaacctggtagacagtccatacatgctcctgggtggcgtcaaaatttcatcaccagagattttgccgaatattacaatcttggaccaccagttgctgctatctacttcaattgccaacgtcaatctggttctgggggaaggagactcatcttataatcatcatcatagagatttatatgtactatacaagtctaagtttcatactaaaataacataagatctttgatggagagtataagtgtttagaacttaggttatcagaatgagattttcataaatagaacgtgaagtatgtatttggttggtagctttatgtatcaagcgatgtcacctttattagaagttcgacattgaatataaataa

SEQ ID NO: 617 - protein, 177 aa, Vigna unguiculata
MRGTTNPLVVGRVIGDVVEPFACSIPLRVVYNHNKEVINSGELKPSQIIIPPRVEVGGDDLRTLYTLVMVDPDAPSPSNPTMREYLHWMVINIPATTGTNFGEEIVSYESPRPTSGIHRLVFVLFKQPGRQSIHAPGWRQNFITRDFAEYYNLGPPVAAIYFNCQRQSGSGGRRLIL

SEQ ID NO: 618 - DNA, 20 bp, Vigna_unguiculata
aataagattgaaataatgcg

SEQ ID NO: 619 - DNA, 20 bp, Vigna_unguiculata
ttacacgtccaacaacaaga

SEQ ID NO: 620 - DNA, 20 bp, Vigna_unguiculata
attacacgtccaacaacaag

SEQ ID NO: 621 - DNA, 20 bp, Vigna_unguiculata
acaacgaaccctcttgttgt

SEQ ID NO: 622 - DNA, 20 bp, Vigna_unguiculata
cttgttgttggacgtgtaat

SEQ ID NO: 623 - DNA, 20 bp, Vigna_unguiculata
tggacgtgtaataggagatg

SEQ ID NO: 624 - DNA, 20 bp, Vigna_unguiculata
acgtgtaataggagatgtgg

SEQ ID NO: 625 - DNA, 20 bp, Vigna_unguiculata
agaggaatcgaacatgcaaa

SEQ ID NO: 626 - DNA, 20 bp, Vigna_unguiculata
tggttgtacacaactctcag

SEQ ID NO: 627 - DNA, 20 bp, Vigna_unguiculata
tgttgatcacttctttgtta

SEQ ID NO: 628 - DNA, 20 bp, Vigna_unguiculata
aacaaagaagtgatcaacag

SEQ ID NO: 629 - DNA, 20 bp, Vigna_unguiculata
ggagggatgattatttggga

SEQ ID NO: 630 - DNA, 20 bp, Vigna_unguiculata
tggagggatgattatttggg

SEQ ID NO: 631 - DNA, 20 bp, Vigna_unguiculata
tcttggagggatgattattt

SEQ ID NO: 632 - DNA, 20 bp, Vigna_unguiculata
ctcttggagggatgattatt

SEQ ID NO: 633 - DNA, 20 bp, Vigna_unguiculata
caccaacctcaactcttgga

SEQ ID NO: 634 - DNA, 20 bp, Vigna_unguiculata
ccaccaacctcaactcttgg

SEQ ID NO: 635 - DNA, 20 bp, Vigna_unguiculata
tctccaccaacctcaactct

SEQ ID NO: 636 - DNA, 20 bp, Vigna_unguiculata
aatcatccctccaagagttg

SEQ ID NO: 637 - DNA, 20 bp, Vigna_unguiculata
atccctccaagagttgaggt

SEQ ID NO: 638 - DNA, 20 bp, Vigna_unguiculata
cctccaagagttgaggttgg

SEQ ID NO: 639 - DNA, 20 bp, Vigna_unguiculata
aggttggtggagatgatctc

SEQ ID NO: 640 - DNA, 20 bp, Vigna_unguiculata
aggttgtaccaaagtgtaaa

SEQ ID NO: 641 - DNA, 20 bp, Vigna_unguiculata
aaggttgtaccaaagtgtaa

SEQ ID NO: 642 - DNA, 20 bp, Vigna_unguiculata
tctcaggaccctttacactt

SEQ ID NO: 643 - DNA, 20 bp, Vigna_unguiculata
ttaggtttaagaaaagtaaa

SEQ ID NO: 644 - DNA, 20 bp, Vigna_unguiculata
aagatgagaagtttgatttt

SEQ ID NO: 645 - DNA, 20 bp, Vigna_unguiculata
tgcatgtatactaatcttgc

SEQ ID NO: 646 - DNA, 20 bp, Vigna_unguiculata
tatactaatcttgcaggtca

SEQ ID NO: 647 - DNA, 20 bp, Vigna_unguiculata
actaatcttgcaggtcatgg

SEQ ID NO: 648 - DNA, 20 bp, Vigna_unguiculata
tacttgggctgggtgcatca

SEQ ID NO: 649 - DNA, 20 bp, Vigna_unguiculata
ttacttgggctgggtgcatc

SEQ ID NO: 650 - DNA, 20 bp, Vigna_unguiculata
atagttgggttacttgggct

SEQ ID NO: 651 - DNA, 20 bp, Vigna_unguiculata
catagttgggttacttgggc

SEQ ID NO: 652 - DNA, 20 bp, Vigna_unguiculata
ccctcatagttgggttactt

SEQ ID NO: 653 - DNA, 20 bp, Vigna_unguiculata
tccctcatagttgggttact

SEQ ID NO: 654 - DNA, 20 bp, Vigna_unguiculata
gaagatattccctcatagtt

SEQ ID NO: 655 - DNA, 20 bp, Vigna_unguiculata
gcccaagtaacccaactatg

SEQ ID NO: 656 - DNA, 20 bp, Vigna_unguiculata
tgaagatattccctcatagt

SEQ ID NO: 657 - DNA, 20 bp, Vigna_unguiculata
cccaagtaacccaactatga

SEQ ID NO: 658 - DNA, 20 bp, Vigna_unguiculata
ctatgagggaatatcttcac

SEQ ID NO: 659 - DNA, 20 bp, Vigna_unguiculata
ttttttaagctatataaatt

SEQ ID NO: 660 - DNA, 20 bp, Vigna_unguiculata
tttttaagctatataaatta

SEQ ID NO: 661 - DNA, 20 bp, Vigna_unguiculata
taaattagggtttaagattt

SEQ ID NO: 662 - DNA, 20 bp, Vigna_unguiculata
aatagatataaatagataga

SEQ ID NO: 663 - DNA, 20 bp, Vigna_unguiculata
tataaatagatagatggtgt

SEQ ID NO: 664 - DNA, 20 bp, Vigna_unguiculata
attcaaaaaccaagacaaag

SEQ ID NO: 665 - DNA, 20 bp, Vigna_unguiculata
taattgtatcctctttgtct

SEQ ID NO: 666 - DNA, 20 bp, Vigna_unguiculata
acgatgtttgtggtgttcaa

SEQ ID NO: 667 - DNA, 20 bp, Vigna_unguiculata
agtgctggtgacgatgtttg

SEQ ID NO: 668 - DNA, 20 bp, Vigna_unguiculata
cacaagcactcacatagtgc

SEQ ID NO: 669 - DNA, 20 bp, Vigna_unguiculata
atgtgagtgcttgtgttttc

SEQ ID NO: 670 - DNA, 20 bp, Vigna_unguiculata
ttcaggtttgttatataccg

SEQ ID NO: 671 - DNA, 20 bp, Vigna_unguiculata
gctgcataaaagaagctcct

SEQ ID NO: 672 - DNA, 20 bp, Vigna_unguiculata
catatatttttcataagttt

SEQ ID NO: 673 - DNA, 20 bp, Vigna_unguiculata
gattcttttattttctgaca

SEQ ID NO: 674 - DNA, 20 bp, Vigna_unguiculata
ctttatgatgtgacttagtg

SEQ ID NO: 675 - DNA, 20 bp, Vigna_unguiculata
aatgatgagagtcaaaaaat

SEQ ID NO: 676 - DNA, 20 bp, Vigna_unguiculata
acttttatacatatatattt

SEQ ID NO: 677 - DNA, 20 bp, Vigna_unguiculata
aaagtcaaagaaaaactaac

SEQ ID NO: 678 - DNA, 20 bp, Vigna_unguiculata
aaattaaaaaatatcatttc

SEQ ID NO: 679 - DNA, 20 bp, Vigna_unguiculata
ttatttttaaatcatgtaat

SEQ ID NO: 680 - DNA, 20 bp, Vigna_unguiculata
aaaagttagaaaatgttttt

SEQ ID NO: 681 - DNA, 20 bp, Vigna_unguiculata
aaaaacattttctaactttt

SEQ ID NO: 682 - DNA, 20 bp, Vigna_unguiculata
aaatttgttttttcaaccca

SEQ ID NO: 683 - DNA, 20 bp, Vigna_unguiculata
tctctcaccctatgatccat

SEQ ID NO: 684 - DNA, 20 bp, Vigna_unguiculata
ctctctcaccctatgatcca

SEQ ID NO: 685 - DNA, 20 bp, Vigna_unguiculata
tttttcaacccatggatcat

SEQ ID NO: 686 - DNA, 20 bp, Vigna_unguiculata
ttttcaacccatggatcata

SEQ ID NO: 687 - DNA, 20 bp, Vigna_unguiculata
gagagagaaaaaaaatagaa

SEQ ID NO: 688 - DNA, 20 bp, Vigna_unguiculata
aaaatagaatggataaagag

SEQ ID NO: 689 - DNA, 20 bp, Vigna_unguiculata
aaatagaatggataaagaga

SEQ ID NO: 690 - DNA, 20 bp, Vigna_unguiculata
aatagaatggataaagagag

SEQ ID NO: 691 - DNA, 20 bp, Vigna_unguiculata
gagaggggtgcgtcatgtca

SEQ ID NO: 692 - DNA, 20 bp, Vigna_unguiculata
tgcgtcatgtcatggcccaa

SEQ ID NO: 693 - DNA, 20 bp, Vigna_unguiculata
gcgtcatgtcatggcccaat

SEQ ID NO: 694 - DNA, 20 bp, Vigna_unguiculata
atacccatcgaaggcccatt

SEQ ID NO: 695 - DNA, 20 bp, Vigna_unguiculata
aatacccatcgaaggcccat

SEQ ID NO: 696 - DNA, 20 bp, Vigna_unguiculata
atggcccaatgggccttcga

SEQ ID NO: 697 - DNA, 20 bp, Vigna_unguiculata
tggcccaatgggccttcgat

SEQ ID NO: 698 - DNA, 20 bp, Vigna_unguiculata
ccgaaacgaatacccatcga

SEQ ID NO: 699 - DNA, 20 bp, Vigna_unguiculata
ccttcgatgggtattcgttt

SEQ ID NO: 700 - DNA, 20 bp, Vigna_unguiculata
aagaagttatgtagttaaat

SEQ ID NO: 701 - DNA, 20 bp, Vigna_unguiculata
attaaactaaaaaggtaaat

SEQ ID NO: 702 - DNA, 20 bp, Vigna_unguiculata
tgatcaaaattaaactaaaa

SEQ ID NO: 703 - DNA, 20 bp, Vigna_unguiculata
aattttgatcatttgtaata

SEQ ID NO: 704 - DNA, 20 bp, Vigna_unguiculata
ctattaaaatctttttgtat

SEQ ID NO: 705 - DNA, 20 bp, Vigna_unguiculata
tattaaaatctttttgtata

SEQ ID NO: 706 - DNA, 20 bp, Vigna_unguiculata
ggaagttatttttctaaaag

SEQ ID NO: 707 - DNA, 20 bp, Vigna_unguiculata
cttacaagttttaagaaaag

SEQ ID NO: 708 - DNA, 20 bp, Vigna_unguiculata
agaacttgttgaaaactaaa

SEQ ID NO: 709 - DNA, 20 bp, Vigna_unguiculata
ttgttgaaaactaaatggat

SEQ ID NO: 710 - DNA, 20 bp, Vigna_unguiculata
tatttttataaattgattat

SEQ ID NO: 711 - DNA, 20 bp, Vigna_unguiculata
aaaagaaaaagtctacgtgt

SEQ ID NO: 712 - DNA, 20 bp, Vigna_unguiculata
caaaagaaaaagtctacgtg

SEQ ID NO: 713 - DNA, 20 bp, Vigna_unguiculata
tggagtgaaagtagaaaaat

SEQ ID NO: 714 - DNA, 20 bp, Vigna_unguiculata
aaagtttaaaactattatat

SEQ ID NO: 715 - DNA, 20 bp, Vigna_unguiculata
tgaaagaaaagaaaatgtta

SEQ ID NO: 716 - DNA, 20 bp, Vigna_unguiculata
tgtgtgtaagactgtttgtc

SEQ ID NO: 717 - DNA, 20 bp, Vigna_unguiculata
cactagtataattgaatcat

SEQ ID NO: 718 - DNA, 20 bp, Vigna_unguiculata
tttaaaaaatgcgtacatac

SEQ ID NO: 719 - DNA, 20 bp, Vigna_unguiculata
atgcgtgatattatattcat

SEQ ID NO: 720 - DNA, 20 bp, Vigna_unguiculata
cagaaaagtttagagaataa

SEQ ID NO: 721 - DNA, 20 bp, Vigna_unguiculata
gaaaaaacttgtaaaaataa

SEQ ID NO: 722 - DNA, 20 bp, Vigna_unguiculata
tgaattagtttggactgaat

SEQ ID NO: 723 - DNA, 20 bp, Vigna_unguiculata
cgtatgaaaatgaattagtt

SEQ ID NO: 724 - DNA, 20 bp, Vigna_unguiculata
tataaaaatgattctaagtt

SEQ ID NO: 725 - DNA, 20 bp, Vigna_unguiculata
gtataaaaatgattctaagt

SEQ ID NO: 726 - DNA, 20 bp, Vigna_unguiculata
tcatttttatacatgtactg

SEQ ID NO: 727 - DNA, 20 bp, Vigna_unguiculata
ttttgattttttttattaat

SEQ ID NO: 728 - DNA, 20 bp, Vigna_unguiculata
ttagattgggttctatcaat

SEQ ID NO: 729 - DNA, 20 bp, Vigna_unguiculata
attgttgagtaaattagatt

SEQ ID NO: 730 - DNA, 20 bp, Vigna_unguiculata
cattgttgagtaaattagat

SEQ ID NO: 731 - DNA, 20 bp, Vigna_unguiculata
caatctaatttactcaacaa

SEQ ID NO: 732 - DNA, 20 bp, Vigna_unguiculata
aatctaatttactcaacaat

SEQ ID NO: 733 - DNA, 20 bp, Vigna_unguiculata
tactcaacaatgggctgagt

SEQ ID NO: 734 - DNA, 20 bp, Vigna_unguiculata
tcattacccaatatcaggtt

SEQ ID NO: 735 - DNA, 20 bp, Vigna_unguiculata
ttcattacccaatatcaggt

SEQ ID NO: 736 - DNA, 20 bp, Vigna_unguiculata
atgtcaacccaacctgatat

SEQ ID NO: 737 - DNA, 20 bp, Vigna_unguiculata
tgtcaacccaacctgatatt

SEQ ID NO: 738 - DNA, 20 bp, Vigna_unguiculata
taaattcattacccaatatc

SEQ ID NO: 739 - DNA, 20 bp, Vigna_unguiculata
gacatatcagagatgttagc

SEQ ID NO: 740 - DNA, 20 bp, Vigna_unguiculata
agacatatcagagatgttag

SEQ ID NO: 741 - DNA, 20 bp, Vigna_unguiculata
atattattgatctatgtcta

SEQ ID NO: 742 - DNA, 20 bp, Vigna_unguiculata
atagttagaaagaaaaaaaa

SEQ ID NO: 743 - DNA, 20 bp, Vigna_unguiculata
taataagtataatttttaaa

SEQ ID NO: 744 - DNA, 20 bp, Vigna_unguiculata
taaaaactaaaaaagattat

SEQ ID NO: 745 - DNA, 20 bp, Vigna_unguiculata
ataatcttttttagttttta

SEQ ID NO: 746 - DNA, 20 bp, Vigna_unguiculata
tattttaaaatatacttaaa

SEQ ID NO: 747 - DNA, 20 bp, Vigna_unguiculata
gtattttaaaatatacttaa

SEQ ID NO: 748 - DNA, 20 bp, Vigna_unguiculata
acatacatttttttaaacaa

SEQ ID NO: 749 - DNA, 20 bp, Vigna_unguiculata
cgctatttctttttcttcga

SEQ ID NO: 750 - DNA, 20 bp, Vigna_unguiculata
tttttcttcgatggatcttt

SEQ ID NO: 751 - DNA, 20 bp, Vigna_unguiculata
tcttcgatggatctttagga

SEQ ID NO: 752 - DNA, 20 bp, Vigna_unguiculata
aagtttgtccctgttgttgc

SEQ ID NO: 753 - DNA, 20 bp, Vigna_unguiculata
atcaacattccagcaacaac

SEQ ID NO: 754 - DNA, 20 bp, Vigna_unguiculata
tcaacattccagcaacaaca

SEQ ID NO: 755 - DNA, 20 bp, Vigna_unguiculata
gcaacaacagggacaaactt

SEQ ID NO: 756 - DNA, 20 bp, Vigna_unguiculata
aaataaaaataaaagagttc

SEQ ID NO: 757 - DNA, 20 bp, Vigna_unguiculata
ataacccaacatatatatga

SEQ ID NO: 758 - DNA, 20 bp, Vigna_unguiculata
attaaccatcatatatatgt

SEQ ID NO: 759 - DNA, 20 bp, Vigna_unguiculata
ttaaccatcatatatatgtt

SEQ ID NO: 760 - DNA, 20 bp, Vigna_unguiculata
tatatattcttttacatata

SEQ ID NO: 761 - DNA, 20 bp, Vigna_unguiculata
taagagattgtaatttcata

SEQ ID NO: 762 - DNA, 20 bp, Vigna_unguiculata
acactattgttttaagattt

SEQ ID NO: 763 - DNA, 20 bp, Vigna_unguiculata
atcttaaaacaatagtgtta

SEQ ID NO: 764 - DNA, 20 bp, Vigna_unguiculata
tcttaaaacaatagtgttat

SEQ ID NO: 765 - DNA, 20 bp, Vigna_unguiculata
tgtgagtcaaagtctaacat

SEQ ID NO: 766 - DNA, 20 bp, Vigna_unguiculata
gtcaaattaatatatattgt

SEQ ID NO: 767 - DNA, 20 bp, Vigna_unguiculata
acaatatatattaatttgac

SEQ ID NO: 768 - DNA, 20 bp, Vigna_unguiculata
tgaatccctgatgttggtct

SEQ ID NO: 769 - DNA, 20 bp, Vigna_unguiculata
gaaagtccaagaccaacatc

SEQ ID NO: 770 - DNA, 20 bp, Vigna_unguiculata
aaagtccaagaccaacatca

SEQ ID NO: 771 - DNA, 20 bp, Vigna_unguiculata
aaacgatgaatccctgatgt

SEQ ID NO: 772 - DNA, 20 bp, Vigna_unguiculata
tttgtgttgtttaaacaacc

SEQ ID NO: 773 - DNA, 20 bp, Vigna_unguiculata
gcatgtatggactgtctacc

SEQ ID NO: 774 - DNA, 20 bp, Vigna_unguiculata
acgccacccaggagcatgta

SEQ ID NO: 775 - DNA, 20 bp, Vigna_unguiculata
agacagtccatacatgctcc

SEQ ID NO: 776 - DNA, 20 bp, Vigna_unguiculata
gacagtccatacatgctcct

SEQ ID NO: 777 - DNA, 20 bp, Vigna_unguiculata
agtccatacatgctcctggg

SEQ ID NO: 778 - DNA, 20 bp, Vigna_unguiculata
atgaaattttgacgccaccc

SEQ ID NO: 779 - DNA, 20 bp, Vigna_unguiculata
gtaatattcggcaaaatctc

SEQ ID NO: 780 - DNA, 20 bp, Vigna_unguiculata
tggtccaagattgtaatatt

SEQ ID NO: 781 - DNA, 20 bp, Vigna_unguiculata
tttgccgaatattacaatct

SEQ ID NO: 782 - DNA, 20 bp, Vigna_unguiculata
aagtagatagcagcaactgg

SEQ ID NO: 783 - DNA, 20 bp, Vigna_unguiculata
ttgaagtagatagcagcaac

SEQ ID NO: 784 - DNA, 20 bp, Vigna_unguiculata
ccccagaaccagattgacgt

SEQ ID NO: 785 - DNA, 20 bp, Vigna_unguiculata
ttcaattgccaacgtcaatc

SEQ ID NO: 786 - DNA, 20 bp, Vigna_unguiculata
tgccaacgtcaatctggttc

SEQ ID NO: 787 - DNA, 20 bp, Vigna_unguiculata
gccaacgtcaatctggttct

SEQ ID NO: 788 - DNA, 20 bp, Vigna_unguiculata
ccaacgtcaatctggttctg

SEQ ID NO: 789 - DNA, 20 bp, Vigna_unguiculata
caacgtcaatctggttctgg

SEQ ID NO: 790 - DNA, 20 bp, Vigna_unguiculata
gtcaatctggttctggggga

SEQ ID NO: 791 - DNA, 20 bp, Vigna_unguiculata
aaataacataagatctttga

SEQ ID NO: 792 - DNA, 20 bp, Vigna_unguiculata
agtataagtgtttagaactt

SEQ ID NO: 793 - DNA, 20 bp, Vigna_unguiculata
tagaacgtgaagtatgtatt

SEQ ID NO: 794 - DNA, 20 bp, Vigna_unguiculata
acgtgaagtatgtatttggt

SEQ ID NO: 795 - DNA, 20 bp, Vigna_unguiculata
caatgtcgaacttctaataa

SEQ ID NO: 796 - DNA, 3720 bp, Vigna unguiculata
cctcgtagcttgtctcataacaaagcaaagtggtattgttgttttgaggaaaggagtgtcctttttcctgattgatgatgcctggaggaagtagggaccctctggttgtagggcgtgtgattggggatgtgttggacccctttgaatgttccattcccatgagggtcacctacaataacagagatatcaccaatggatgtgaattcaaaccttcacaagttttcaaccaaccaagaataactatcggtggagatgatctcagaaacttctacaccttggtaattcattcttcttcttcttcttcttttgtttcttcctcctcatcttcaactagttgtgttcaccactcactgcaatatataatttatacaatcattaatatttatttatatgaaataaaatgtcataaaatgatatttgtaaatattaaaatggatgttaatatatgtaaattaagtaattaaaattatttttcgttcatatattttgaaaggatgaactttcacatattttaatttctttcatttacagatcgcagttgatcccgatgcacctagtccaagtgaccctaatttgagggaatacctccattggtgagccaaaaattgttcgaatacaccatgaatctttgtttatatgaactgaattatacatttaagattttttcaagcttaatgctgcaatatttaaccacctctctctgtctctctctcaaacgtacttcaggttggtcactgatattcctgcaacaacgggacctagtttcggtatgtatgatatttttcacttaaacttgatgattcttttatgtatgtgtatattaataacaaaatttatgttttcttcagtgtatttgtgatttatggtttctttaagttactactaacaggaactcaaattttcaaactattagttatatttcattcaactctgaattcctaaactcgtttgacctagttgtggaataaatatgtcggtcatatgataagaagtctctaccaataataattattttacagagtatactgatgggaaataatttggatattctatacaagaatccatcagcttttatctcactcaaaagtttttctttttgtcgttaggagtagtacgtatcctttctcaaaatatgaatcatgtctcagttcatcagaatatatggtccaaaatagtacatataaacatgttcctatgtttttaatacaataaacattattcttgacatttatattttaatttccctagttaatatttattcgtattgttatagggttcagggtatttttctttaattaaatcttcagtattataattataaataattgtaattggtttcatcttcctagtttacatttgcctttggattgaatttgatctaaaggaatcttgaattcatttaacataagtgataagtgagttcgttacttatggatataggaatactaaatgtgagtgtggaacatatagtaaatgtgaaaaaaaaaagtgtttattttgaagcacggtttgtgttgcaacttttatatattatcaactaattagaacttattcctgatatgattttaagattattatttaaaaattaataagttattcaagcataaaatttgctgtcggatcatgatataaaattattcacactattattaatagaactctaacaacttaagatatgacttgaaacaatgtaattagaaaacactagtggatgaaaatcatccctaattattggttttgatgcttaaaatttaatttttttcaaaaggaattctaatgattactttgtgaaattatgacatgcatgcatatgttacatcaaccatattctaaaatattatgttatgtgagataggtagataggcatatataatatatttaaataaattcaaattaagaggattaaagttaaaaaaaaaaaatagagatgatgcattaagtcatttttacaactttgggtgataataagagcatgttttgggtgataataacaagattcagaatatacagtagttggagaatccaaattagccaatcaaactacgaatcataaaagcattgaagccttgtttgttttctctcttccatcagaaccacagagccatcgaattcttaaagtgcataatcatgtgagaaaagatagaagattctattcaagaaacgacacgaaaagcaactgttctgactgaaggagaatattcatattgaatagattaccttttgttggccatctttgcagatcacatgacaagataacaccaaaaccgtcatagctatagggtctatagatcagaaaagaacatcatacaaaaagctgcataacctgttcatgcaactggtttaacaaaatctgacaaggtgaagaggtaccataattaagaatcatatatgtttaaattcaactccaaacatatcttcaccaaaccgcagttcgagtcaattagggttaccgtgagagactaaactgtccttcttagtttcattcatagtattcacaattcaaactcaagacctgtgattgagttagaacaaactctacacaatttgattcatatgggtttggtgattccttaacaacatatataattcattaaaataaacatctttaaaaatttcgaagcttttcacctccaccaaaccccatcaaccttctcaccaggatctgcaatcttcaaatccttctcatgaaatcagaaacaatcccttgaaaaacttttgaatctaccagatttagtgaattgctggaagaaaaagaaaataaataaataaatctacttgtagcacctaccagcttatcaaaatcagagcttaccggtaggagtgatgaatgtttcttccctccgctctaagatatttccacagaattgaaatgacgcgttttatttaatttcaatttttatcgcttcaattagaaatcgacacgtaactctcttctgataataattgttattattatttatcattattttaagattttctagtattttctaattggaagatagcgaaagggacataacattagtggctttgcttttctgaaaaaaggtagacgaattttcatccactcgtaggttctttgaagtctttttataagcagtactagtaggaccttatatgtgttattaatatctgaactgaaatatactatttttattgttgataatgagcctgaaatttacactcgtaatacaaattatttgcacaggtcatgaggttgtaacatacgagagtccacgacccatgatggggattcatcgtatcgtgtttgtgttgtttcggcaactgggtcgggaaactgtgtatgcaccagggtggcgccagaatttcaacaccagagaatttgctgaactttacaaccttggattgcctgttgcggctgtctatttcaacattcagagggaatctggttctggtggaaggaggttatattagaaaaccttatagtatggaacaaaataaagtactaattaaacttccttgagggtttcggaaatattcgcgatgatcacctagtaaaatgagtgcctctttaacttaaactcaacatatataccaaataaatcactagctcggttgggtagcaccaagaaatctatatgtatccatgaatggaacccaagtatccttcggggttcattttttttttttttttcatttatgtattttgtatttaaaatattttatattataaaagtgtgttagaaatacttatgttcgatt

SEQ ID NO: 797 - protein, 177 aa, Vigna unguiculata
MMPGGSRDPLVVGRVIGDVLDPFECSIPMRVTYNNRDITNGCEFKPSQVFNQPRITIGGDDLRNFYTLIAVDPDAPSPSDPNLREYLHWLVTDIPATTGPSFGHEVVTYESPRPMMGIHRIVFVLFRQLGRETVYAPGWRQNFNTREFAELYNLGLPVAAVYFNIQRESGSGGRRLY

SEQ ID NO: 798 - DNA, 20 bp, Vigna_unguiculata
ttgttatgagacaagctacg

SEQ ID NO: 799 - DNA, 20 bp, Vigna_unguiculata
tgtctcataacaaagcaaag

SEQ ID NO: 800 - DNA, 20 bp, Vigna_unguiculata
aaagtggtattgttgttttg

SEQ ID NO: 801 - DNA, 20 bp, Vigna_unguiculata
ggtattgttgttttgaggaa

SEQ ID NO: 802 - DNA, 20 bp, Vigna_unguiculata
ggcatcatcaatcaggaaaa

SEQ ID NO: 803 - DNA, 20 bp, Vigna_unguiculata
tcctccaggcatcatcaatc

SEQ ID NO: 804 - DNA, 20 bp, Vigna_unguiculata
ttttcctgattgatgatgcc

SEQ ID NO: 805 - DNA, 20 bp, Vigna_unguiculata
tcctgattgatgatgcctgg

SEQ ID NO: 806 - DNA, 20 bp, Vigna_unguiculata
agagggtccctacttcctcc

SEQ ID NO: 807 - DNA, 20 bp, Vigna_unguiculata
tgatgatgcctggaggaagt

SEQ ID NO: 808 - DNA, 20 bp, Vigna_unguiculata
gatgatgcctggaggaagta

SEQ ID NO: 809 - DNA, 20 bp, Vigna_unguiculata
tggaggaagtagggaccctc

SEQ ID NO: 810 - DNA, 20 bp, Vigna_unguiculata
tcacacgccctacaaccaga

SEQ ID NO: 811 - DNA, 20 bp, Vigna_unguiculata
atcacacgccctacaaccag

SEQ ID NO: 812 - DNA, 20 bp, Vigna_unguiculata
agtagggaccctctggttgt

SEQ ID NO: 813 - DNA, 20 bp, Vigna_unguiculata
gtagggaccctctggttgta

SEQ ID NO: 814 - DNA, 20 bp, Vigna_unguiculata
ctggttgtagggcgtgtgat

SEQ ID NO: 815 - DNA, 20 bp, Vigna_unguiculata
tggttgtagggcgtgtgatt

SEQ ID NO: 816 - DNA, 20 bp, Vigna_unguiculata
ggttgtagggcgtgtgattg

SEQ ID NO: 817 - DNA, 20 bp, Vigna_unguiculata
gcgtgtgattggggatgtgt

SEQ ID NO: 818 - DNA, 20 bp, Vigna_unguiculata
tgggaatggaacattcaaag

SEQ ID NO: 819 - DNA, 20 bp, Vigna_unguiculata
atgggaatggaacattcaaa

SEQ ID NO: 820 - DNA, 20 bp, Vigna_unguiculata
catgggaatggaacattcaa

SEQ ID NO: 821 - DNA, 20 bp, Vigna_unguiculata
gtaggtgaccctcatgggaa

SEQ ID NO: 822 - DNA, 20 bp, Vigna_unguiculata
ttgaatgttccattcccatg

SEQ ID NO: 823 - DNA, 20 bp, Vigna_unguiculata
tgaatgttccattcccatga

SEQ ID NO: 824 - DNA, 20 bp, Vigna_unguiculata
ttattgtaggtgaccctcat

SEQ ID NO: 825 - DNA, 20 bp, Vigna_unguiculata
gttattgtaggtgaccctca

SEQ ID NO: 826 - DNA, 20 bp, Vigna_unguiculata
ggtgatatctctgttattgt

SEQ ID NO: 827 - DNA, 20 bp, Vigna_unguiculata
aataacagagatatcaccaa

SEQ ID NO: 828 - DNA, 20 bp, Vigna_unguiculata
aggtttgaattcacatccat

SEQ ID NO: 829 - DNA, 20 bp, Vigna_unguiculata
ggttggttgaaaacttgtga

SEQ ID NO: 830 - DNA, 20 bp, Vigna_unguiculata
caccgatagttattcttggt

SEQ ID NO: 831 - DNA, 20 bp, Vigna_unguiculata
tctccaccgatagttattct

SEQ ID NO: 832 - DNA, 20 bp, Vigna_unguiculata
aaccaaccaagaataactat

SEQ ID NO: 833 - DNA, 20 bp, Vigna_unguiculata
caaccaagaataactatcgg

SEQ ID NO: 834 - DNA, 20 bp, Vigna_unguiculata
tctcagaaacttctacacct

SEQ ID NO: 835 - DNA, 20 bp, Vigna_unguiculata
agaagaagaatgaattacca

SEQ ID NO: 836 - DNA, 20 bp, Vigna_unguiculata
acaactagttgaagatgagg

SEQ ID NO: 837 - DNA, 20 bp, Vigna_unguiculata
aacacaactagttgaagatg

SEQ ID NO: 838 - DNA, 20 bp, Vigna_unguiculata
aaattatatattgcagtgag

SEQ ID NO: 839 - DNA, 20 bp, Vigna_unguiculata
gatatttgtaaatattaaaa

SEQ ID NO: 840 - DNA, 20 bp, Vigna_unguiculata
tttcgttcatatattttgaa

SEQ ID NO: 841 - DNA, 20 bp, Vigna_unguiculata
tcacttggactaggtgcatc

SEQ ID NO: 842 - DNA, 20 bp, Vigna_unguiculata
gtcacttggactaggtgcat

SEQ ID NO: 843 - DNA, 20 bp, Vigna_unguiculata
aaattagggtcacttggact

SEQ ID NO: 844 - DNA, 20 bp, Vigna_unguiculata
tccctcaaattagggtcact

SEQ ID NO: 845 - DNA, 20 bp, Vigna_unguiculata
ggaggtattccctcaaatta

SEQ ID NO: 846 - DNA, 20 bp, Vigna_unguiculata
gtccaagtgaccctaatttg

SEQ ID NO: 847 - DNA, 20 bp, Vigna_unguiculata
tggaggtattccctcaaatt

SEQ ID NO: 848 - DNA, 20 bp, Vigna_unguiculata
tccaagtgaccctaatttga

SEQ ID NO: 849 - DNA, 20 bp, Vigna_unguiculata
atttgagggaatacctccat

SEQ ID NO: 850 - DNA, 20 bp, Vigna_unguiculata
aatttttggctcaccaatgg

SEQ ID NO: 851 - DNA, 20 bp, Vigna_unguiculata
aacaatttttggctcaccaa

SEQ ID NO: 852 - DNA, 20 bp, Vigna_unguiculata
tggtgtattcgaacaatttt

SEQ ID NO: 853 - DNA, 20 bp, Vigna_unguiculata
ttcatataaacaaagattca

SEQ ID NO: 854 - DNA, 20 bp, Vigna_unguiculata
gagagagagacagagagagg

SEQ ID NO: 855 - DNA, 20 bp, Vigna_unguiculata
tttgagagagagacagagag

SEQ ID NO: 856 - DNA, 20 bp, Vigna_unguiculata
ctctctctcaaacgtacttc

SEQ ID NO: 857 - DNA, 20 bp, Vigna_unguiculata
ctctcaaacgtacttcaggt

SEQ ID NO: 858 - DNA, 20 bp, Vigna_unguiculata
aaactaggtcccgttgttgc

SEQ ID NO: 859 - DNA, 20 bp, Vigna_unguiculata
cactgatattcctgcaacaa

SEQ ID NO: 860 - DNA, 20 bp, Vigna_unguiculata
actgatattcctgcaacaac

SEQ ID NO: 861 - DNA, 20 bp, Vigna_unguiculata
gcaacaacgggacctagttt

SEQ ID NO: 862 - DNA, 20 bp, Vigna_unguiculata
atatcatacataccgaaact

SEQ ID NO: 863 - DNA, 20 bp, Vigna_unguiculata
ttcagtgtatttgtgattta

SEQ ID NO: 864 - DNA, 20 bp, Vigna_unguiculata
ttctttaagttactactaac

SEQ ID NO: 865 - DNA, 20 bp, Vigna_unguiculata
caactaggtcaaacgagttt

SEQ ID NO: 866 - DNA, 20 bp, Vigna_unguiculata
aaactcgtttgacctagttg

SEQ ID NO: 867 - DNA, 20 bp, Vigna_unguiculata
gacatatttattccacaact

SEQ ID NO: 868 - DNA, 20 bp, Vigna_unguiculata
tagttgtggaataaatatgt

SEQ ID NO: 869 - DNA, 20 bp, Vigna_unguiculata
ctctgtaaaataattattat

SEQ ID NO: 870 - DNA, 20 bp, Vigna_unguiculata
tattttacagagtatactga

SEQ ID NO: 871 - DNA, 20 bp, Vigna_unguiculata
attttacagagtatactgat

SEQ ID NO: 872 - DNA, 20 bp, Vigna_unguiculata
tatactgatgggaaataatt

SEQ ID NO: 873 - DNA, 20 bp, Vigna_unguiculata
ttgagtgagataaaagctga

SEQ ID NO: 874 - DNA, 20 bp, Vigna_unguiculata
aagtttttctttttgtcgtt

SEQ ID NO: 875 - DNA, 20 bp, Vigna_unguiculata
catgattcatattttgagaa

SEQ ID NO: 876 - DNA, 20 bp, Vigna_unguiculata
tctcagttcatcagaatata

SEQ ID NO: 877 - DNA, 20 bp, Vigna_unguiculata
catgtttatatgtactattt

SEQ ID NO: 878 - DNA, 20 bp, Vigna_unguiculata
gtttattgtattaaaaacat

SEQ ID NO: 879 - DNA, 20 bp, Vigna_unguiculata
atacgaataaatattaacta

SEQ ID NO: 880 - DNA, 20 bp, Vigna_unguiculata
aatacgaataaatattaact

SEQ ID NO: 881 - DNA, 20 bp, Vigna_unguiculata
atatttattcgtattgttat

SEQ ID NO: 882 - DNA, 20 bp, Vigna_unguiculata
tatttattcgtattgttata

SEQ ID NO: 883 - DNA, 20 bp, Vigna_unguiculata
ttcgtattgttatagggttc

SEQ ID NO: 884 - DNA, 20 bp, Vigna_unguiculata
tcgtattgttatagggttca

SEQ ID NO: 885 - DNA, 20 bp, Vigna_unguiculata
taattataaataattgtaat

SEQ ID NO: 886 - DNA, 20 bp, Vigna_unguiculata
tccaaaggcaaatgtaaact

SEQ ID NO: 887 - DNA, 20 bp, Vigna_unguiculata
tcctagtttacatttgcctt

SEQ ID NO: 888 - DNA, 20 bp, Vigna_unguiculata
ttagatcaaattcaatccaa

SEQ ID NO: 889 - DNA, 20 bp, Vigna_unguiculata
ttggattgaatttgatctaa

SEQ ID NO: 890 - DNA, 20 bp, Vigna_unguiculata
ataagtgagttcgttactta

SEQ ID NO: 891 - DNA, 20 bp, Vigna_unguiculata
agttcgttacttatggatat

SEQ ID NO: 892 - DNA, 20 bp, Vigna_unguiculata
aggaatactaaatgtgagtg

SEQ ID NO: 893 - DNA, 20 bp, Vigna_unguiculata
aaagtgtttattttgaagca

SEQ ID NO: 894 - DNA, 20 bp, Vigna_unguiculata
aataatcttaaaatcatatc

SEQ ID NO: 895 - DNA, 20 bp, Vigna_unguiculata
tcaagcataaaatttgctgt

SEQ ID NO: 896 - DNA, 20 bp, Vigna_unguiculata
atgtaattagaaaacactag

SEQ ID NO: 897 - DNA, 20 bp, Vigna_unguiculata
agcatcaaaaccaataatta

SEQ ID NO: 898 - DNA, 20 bp, Vigna_unguiculata
tgaaaatcatccctaattat

SEQ ID NO: 899 - DNA, 20 bp, Vigna_unguiculata
aagcatcaaaaccaataatt

SEQ ID NO: 900 - DNA, 20 bp, Vigna_unguiculata
aaaatttaatttttttcaaa

SEQ ID NO: 901 - DNA, 20 bp, Vigna_unguiculata
taacataatattttagaata

SEQ ID NO: 902 - DNA, 20 bp, Vigna_unguiculata
aatattatgttatgtgagat

SEQ ID NO: 903 - DNA, 20 bp, Vigna_unguiculata
gttatgtgagataggtagat

SEQ ID NO: 904 - DNA, 20 bp, Vigna_unguiculata
ttaaataaattcaaattaag

SEQ ID NO: 905 - DNA, 20 bp, Vigna_unguiculata
ttaagtcatttttacaactt

SEQ ID NO: 906 - DNA, 20 bp, Vigna_unguiculata
taagtcatttttacaacttt

SEQ ID NO: 907 - DNA, 20 bp, Vigna_unguiculata
gtgataataagagcatgttt

SEQ ID NO: 908 - DNA, 20 bp, Vigna_unguiculata
tgataataagagcatgtttt

SEQ ID NO: 909 - DNA, 20 bp, Vigna_unguiculata
gattcagaatatacagtagt

SEQ ID NO: 910 - DNA, 20 bp, Vigna_unguiculata
cgtagtttgattggctaatt

SEQ ID NO: 911 - DNA, 20 bp, Vigna_unguiculata
tttatgattcgtagtttgat

SEQ ID NO: 912 - DNA, 20 bp, Vigna_unguiculata
tggaagagagaaaacaaaca

SEQ ID NO: 913 - DNA, 20 bp, Vigna_unguiculata
cgatggctctgtggttctga

SEQ ID NO: 914 - DNA, 20 bp, Vigna_unguiculata
ttaagaattcgatggctctg

SEQ ID NO: 915 - DNA, 20 bp, Vigna_unguiculata
tatgcactttaagaattcga

SEQ ID NO: 916 - DNA, 20 bp, Vigna_unguiculata
aaagcaactgttctgactga

SEQ ID NO: 917 - DNA, 20 bp, Vigna_unguiculata
ttgaatagattaccttttgt

SEQ ID NO: 918 - DNA, 20 bp, Vigna_unguiculata
ctgcaaagatggccaacaaa

SEQ ID NO: 919 - DNA, 20 bp, Vigna_unguiculata
tgtcatgtgatctgcaaaga

SEQ ID NO: 920 - DNA, 20 bp, Vigna_unguiculata
ccctatagctatgacggttt

SEQ ID NO: 921 - DNA, 20 bp, Vigna_unguiculata
tatagaccctatagctatga

SEQ ID NO: 922 - DNA, 20 bp, Vigna_unguiculata
accaaaaccgtcatagctat

SEQ ID NO: 923 - DNA, 20 bp, Vigna_unguiculata
ccaaaaccgtcatagctata

SEQ ID NO: 924 - DNA, 20 bp, Vigna_unguiculata
gttaaaccagttgcatgaac

SEQ ID NO: 925 - DNA, 20 bp, Vigna_unguiculata
gcataacctgttcatgcaac

SEQ ID NO: 926 - DNA, 20 bp, Vigna_unguiculata
tggtttaacaaaatctgaca

SEQ ID NO: 927 - DNA, 20 bp, Vigna_unguiculata
caaaatctgacaaggtgaag

SEQ ID NO: 928 - DNA, 20 bp, Vigna_unguiculata
acatatatgattcttaatta

SEQ ID NO: 929 - DNA, 20 bp, Vigna_unguiculata
cggtttggtgaagatatgtt

SEQ ID NO: 930 - DNA, 20 bp, Vigna_unguiculata
aattgactcgaactgcggtt

SEQ ID NO: 931 - DNA, 20 bp, Vigna_unguiculata
accctaattgactcgaactg

SEQ ID NO: 932 - DNA, 20 bp, Vigna_unguiculata
aaccgcagttcgagtcaatt

SEQ ID NO: 933 - DNA, 20 bp, Vigna_unguiculata
accgcagttcgagtcaatta

SEQ ID NO: 934 - DNA, 20 bp, Vigna_unguiculata
aaggacagtttagtctctca

SEQ ID NO: 935 - DNA, 20 bp, Vigna_unguiculata
tactatgaatgaaactaaga

SEQ ID NO: 936 - DNA, 20 bp, Vigna_unguiculata
gtttgttctaactcaatcac

SEQ ID NO: 937 - DNA, 20 bp, Vigna_unguiculata
tctacacaatttgattcata

SEQ ID NO: 938 - DNA, 20 bp, Vigna_unguiculata
ctacacaatttgattcatat

SEQ ID NO: 939 - DNA, 20 bp, Vigna_unguiculata
caatttgattcatatgggtt

SEQ ID NO: 940 - DNA, 20 bp, Vigna_unguiculata
atgaattatatatgttgtta

SEQ ID NO: 941 - DNA, 20 bp, Vigna_unguiculata
aggttgatggggtttggtgg

SEQ ID NO: 942 - DNA, 20 bp, Vigna_unguiculata
agaaggttgatggggtttgg

SEQ ID NO: 943 - DNA, 20 bp, Vigna_unguiculata
gtgagaaggttgatggggtt

SEQ ID NO: 944 - DNA, 20 bp, Vigna_unguiculata
tcctggtgagaaggttgatg

SEQ ID NO: 945 - DNA, 20 bp, Vigna_unguiculata
atcctggtgagaaggttgat

SEQ ID NO: 946 - DNA, 20 bp, Vigna_unguiculata
gatcctggtgagaaggttga

SEQ ID NO: 947 - DNA, 20 bp, Vigna_unguiculata
gattgcagatcctggtgaga

SEQ ID NO: 948 - DNA, 20 bp, Vigna_unguiculata
accccatcaaccttctcacc

SEQ ID NO: 949 - DNA, 20 bp, Vigna_unguiculata
gatttgaagattgcagatcc

SEQ ID NO: 950 - DNA, 20 bp, Vigna_unguiculata
ttgtttctgatttcatgaga

SEQ ID NO: 951 - DNA, 20 bp, Vigna_unguiculata
tagattcaaaagtttttcaa

SEQ ID NO: 952 - DNA, 20 bp, Vigna_unguiculata
gtagattcaaaagtttttca

SEQ ID NO: 953 - DNA, 20 bp, Vigna_unguiculata
ttccagcaattcactaaatc

SEQ ID NO: 954 - DNA, 20 bp, Vigna_unguiculata
taccagatttagtgaattgc

SEQ ID NO: 955 - DNA, 20 bp, Vigna_unguiculata
tctgattttgataagctggt

SEQ ID NO: 956 - DNA, 20 bp, Vigna_unguiculata
aagctctgattttgataagc

SEQ ID NO: 957 - DNA, 20 bp, Vigna_unguiculata
ttatcaaaatcagagcttac

SEQ ID NO: 958 - DNA, 20 bp, Vigna_unguiculata
caaaatcagagcttaccggt

SEQ ID NO: 959 - DNA, 20 bp, Vigna_unguiculata
gaaacattcatcactcctac

SEQ ID NO: 960 - DNA, 20 bp, Vigna_unguiculata
tggaaatatcttagagcgga

SEQ ID NO: 961 - DNA, 20 bp, Vigna_unguiculata
gtggaaatatcttagagcgg

SEQ ID NO: 962 - DNA, 20 bp, Vigna_unguiculata
tctgtggaaatatcttagag

SEQ ID NO: 963 - DNA, 20 bp, Vigna_unguiculata
aacgcgtcatttcaattctg

SEQ ID NO: 964 - DNA, 20 bp, Vigna_unguiculata
attttctagtattttctaat

SEQ ID NO: 965 - DNA, 20 bp, Vigna_unguiculata
tctaattggaagatagcgaa

SEQ ID NO: 966 - DNA, 20 bp, Vigna_unguiculata
ctaattggaagatagcgaaa

SEQ ID NO: 967 - DNA, 20 bp, Vigna_unguiculata
cgaaagggacataacattag

SEQ ID NO: 968 - DNA, 20 bp, Vigna_unguiculata
ggctttgcttttctgaaaaa

SEQ ID NO: 969 - DNA, 20 bp, Vigna_unguiculata
acgaattttcatccactcgt

SEQ ID NO: 970 - DNA, 20 bp, Vigna_unguiculata
gacttcaaagaacctacgag

SEQ ID NO: 971 - DNA, 20 bp, Vigna_unguiculata
tttttataagcagtactagt

SEQ ID NO: 972 - DNA, 20 bp, Vigna_unguiculata
agatattaataacacatata

SEQ ID NO: 973 - DNA, 20 bp, Vigna_unguiculata
gtattacgagtgtaaatttc

SEQ ID NO: 974 - DNA, 20 bp, Vigna_unguiculata
gtaatacaaattatttgcac

SEQ ID NO: 975 - DNA, 20 bp, Vigna_unguiculata
aattatttgcacaggtcatg

SEQ ID NO: 976 - DNA, 20 bp, Vigna_unguiculata
tgaatccccatcatgggtcg

SEQ ID NO: 977 - DNA, 20 bp, Vigna_unguiculata
cgagagtccacgacccatga

SEQ ID NO: 978 - DNA, 20 bp, Vigna_unguiculata
gagagtccacgacccatgat

SEQ ID NO: 979 - DNA, 20 bp, Vigna_unguiculata
agagtccacgacccatgatg

SEQ ID NO: 980 - DNA, 20 bp, Vigna_unguiculata
atacgatgaatccccatcat

SEQ ID NO: 981 - DNA, 20 bp, Vigna_unguiculata
gatacgatgaatccccatca

SEQ ID NO: 982 - DNA, 20 bp, Vigna_unguiculata
gtatcgtgtttgtgttgttt

SEQ ID NO: 983 - DNA, 20 bp, Vigna_unguiculata
gtttgtgttgtttcggcaac

SEQ ID NO: 984 - DNA, 20 bp, Vigna_unguiculata
tttgtgttgtttcggcaact

SEQ ID NO: 985 - DNA, 20 bp, Vigna_unguiculata
tgttgtttcggcaactgggt

SEQ ID NO: 986 - DNA, 20 bp, Vigna_unguiculata
gttgtttcggcaactgggtc

SEQ ID NO: 987 - DNA, 20 bp, Vigna_unguiculata
cgggaaactgtgtatgcacc

SEQ ID NO: 988 - DNA, 20 bp, Vigna_unguiculata
gggaaactgtgtatgcacca

SEQ ID NO: 989 - DNA, 20 bp, Vigna_unguiculata
aaactgtgtatgcaccaggg

SEQ ID NO: 990 - DNA, 20 bp, Vigna_unguiculata
ttgaaattctggcgccaccc

SEQ ID NO: 991 - DNA, 20 bp, Vigna_unguiculata
attctctggtgttgaaattc

SEQ ID NO: 992 - DNA, 20 bp, Vigna_unguiculata
gtaaagttcagcaaattctc

SEQ ID NO: 993 - DNA, 20 bp, Vigna_unguiculata
tttgctgaactttacaacct

SEQ ID NO: 994 - DNA, 20 bp, Vigna_unguiculata
cagccgcaacaggcaatcca

SEQ ID NO: 995 - DNA, 20 bp, Vigna_unguiculata
caaccttggattgcctgttg

SEQ ID NO: 996 - DNA, 20 bp, Vigna_unguiculata
ttgaaatagacagccgcaac

SEQ ID NO: 997 - DNA, 20 bp, Vigna_unguiculata
ctgtctatttcaacattcag

SEQ ID NO: 998 - DNA, 20 bp, Vigna_unguiculata
tgtctatttcaacattcaga

SEQ ID NO: 999 - DNA, 20 bp, Vigna_unguiculata
ttcaacattcagagggaatc

SEQ ID NO: 1000 - DNA, 20 bp, Vigna_unguiculata
attcagagggaatctggttc

SEQ ID NO: 1001 - DNA, 20 bp, Vigna_unguiculata
cagagggaatctggttctgg

SEQ ID NO: 1002 - DNA, 20 bp, Vigna_unguiculata
gggaatctggttctggtgga

SEQ ID NO: 1003 - DNA, 20 bp, Vigna_unguiculata
aatctggttctggtggaagg

SEQ ID NO: 1004 - DNA, 20 bp, Vigna_unguiculata
ttattttgttccatactata

SEQ ID NO: 1005 - DNA, 20 bp, Vigna_unguiculata
tattagaaaaccttatagta

SEQ ID NO: 1006 - DNA, 20 bp, Vigna_unguiculata
gtactaattaaacttccttg

SEQ ID NO: 1007 - DNA, 20 bp, Vigna_unguiculata
tactaattaaacttccttga

SEQ ID NO: 1008 - DNA, 20 bp, Vigna_unguiculata
gaatatttccgaaaccctca

SEQ ID NO: 1009 - DNA, 20 bp, Vigna_unguiculata
ttaaacttccttgagggttt

SEQ ID NO: 1010 - DNA, 20 bp, Vigna_unguiculata
aaagaggcactcattttact

SEQ ID NO: 1011 - DNA, 20 bp, Vigna_unguiculata
atgttgagtttaagttaaag

SEQ ID NO: 1012 - DNA, 20 bp, Vigna_unguiculata
aaccgagctagtgatttatt

SEQ ID NO: 1013 - DNA, 20 bp, Vigna_unguiculata
taccaaataaatcactagct

SEQ ID NO: 1014 - DNA, 20 bp, Vigna_unguiculata
aaataaatcactagctcggt

SEQ ID NO: 1015 - DNA, 20 bp, Vigna_unguiculata
aataaatcactagctcggtt

SEQ ID NO: 1016 - DNA, 20 bp, Vigna_unguiculata
atggatacatatagatttct

SEQ ID NO: 1017 - DNA, 20 bp, Vigna_unguiculata
aatctatatgtatccatgaa

SEQ ID NO: 1018 - DNA, 20 bp, Vigna_unguiculata
ggatacttgggttccattca

SEQ ID NO: 1019 - DNA, 20 bp, Vigna_unguiculata
atgaaccccgaaggatactt

SEQ ID NO: 1020 - DNA, 20 bp, Vigna_unguiculata
aatgaaccccgaaggatact

SEQ ID NO: 1021 - DNA, 20 bp, Vigna_unguiculata
aatggaacccaagtatcctt

SEQ ID NO: 1022 - DNA, 20 bp, Vigna_unguiculata
atggaacccaagtatccttc

SEQ ID NO: 1023 - DNA, 20 bp, Vigna_unguiculata
tggaacccaagtatccttcg

SEQ ID NO: 1024 - DNA, 20 bp, Vigna_unguiculata
aaaaaaaaaatgaaccccga

SEQ ID NO: 1025 - DNA, 1239 bp, Arachis hypogaea
atcacattcacacatgcatacatagattggagcaattatataactttgtattgaagaagtaactttaccttacattgaataagtaactaataagaaaaataattgatgatgatggcttcatcaaatgcattttcatcatcagatccacttactataggaagggtgatcggagatgtagtcgatgatttcacagaaactgtgaaaatgacagtcacttacaacaacaagcatgtctacaatggctatgagttcttcccttcttcagtttccactaaacctagggttcacattcatggaggcgacttgagatcctctttcactctggttcattaattattaataattttattacttaatatgttatatactagttatttcatgattatcatacctttactttcttttgtatttgtttgtttcagattatgacagatccagatgttcctggtcctagtgacccatacttaagagaacacttacactggtactttatatacatgcatgcactttgatttgatatgattaatttgttcaactacttaattaaacgatggatcttattagttgttatagtattaggaattattagttgtatatgtttgttttggggcacataattcacaagaattaattttaggtaacaaattgtatacttttatttttaagacatttaagttagttaattagttaatgtgatttgcataaaataattaaatatttaatttcttattataggatggtgactgatatcccaggcacaacggatgcaacatttggtaagatatagttacttccttaattagctttcacagtattataatgaatgtatgtttttcatgtaattaaaaaacacatacaggaaaggaggtgataaagtatgagatgccaaagccaaacataggaatacatagatttgttttggtgctttacaagcagaagaggaggcagacggtgaacaagattcccagttcaagggacctcttcaacacaaggaaattcgcagttgaaaatgatcttggcactcctgtggctgctgtcttcttcaatgctcaaagagaaaccgcttgtagaaagcgctgatcagctgatctttaaaacaaaataatcttttatcttaagtgtgtgcaactcatgtagtagtggcactattatggtgttttacttatatataatatggtatgggatccgccaagaataagtttaagactgttaaggttatgggtatccatctcctaaata

SEQ ID NO: 1026 - protein, 180 aa, Arachis hypogaea
MMMASSNAFSSSDPLTIGRVIGDVVDDFTETVKMTVTYNNKHVYNGYEFFPSSVSTKPRVHIHGGDLRSSFTLIMTDPDVPGPSDPYLREHLHWMVTDIPGTTDATFGKEVIKYEMPKPNIGIHRFVLVLYKQKRRQTVNKIPSSRDLFNTRKFAVENDLGTPVAAVFFNAQRETACRKR

SEQ ID NO: 1027 - DNA, 20 bp, Arachis_hypogaea
tcacacatgcatacatagat

SEQ ID NO: 1028 - DNA, 20 bp, Arachis_hypogaea
ttagttacttattcaatgta

SEQ ID NO: 1029 - DNA, 20 bp, Arachis_hypogaea
agaaaaataattgatgatga

SEQ ID NO: 1030 - DNA, 20 bp, Arachis_hypogaea
atcacccttcctatagtaag

SEQ ID NO: 1031 - DNA, 20 bp, Arachis_hypogaea
tcatcagatccacttactat

SEQ ID NO: 1032 - DNA, 20 bp, Arachis_hypogaea
cagatccacttactatagga

SEQ ID NO: 1033 - DNA, 20 bp, Arachis_hypogaea
agatccacttactataggaa

SEQ ID NO: 1034 - DNA, 20 bp, Arachis_hypogaea
cttactataggaagggtgat

SEQ ID NO: 1035 - DNA, 20 bp, Arachis_hypogaea
aacaacaagcatgtctacaa

SEQ ID NO: 1036 - DNA, 20 bp, Arachis_hypogaea
gtttagtggaaactgaagaa

SEQ ID NO: 1037 - DNA, 20 bp, Arachis_hypogaea
ggtttagtggaaactgaaga

SEQ ID NO: 1038 - DNA, 20 bp, Arachis_hypogaea
aatgtgaaccctaggtttag

SEQ ID NO: 1039 - DNA, 20 bp, Arachis_hypogaea
cttcagtttccactaaacct

SEQ ID NO: 1040 - DNA, 20 bp, Arachis_hypogaea
ttcagtttccactaaaccta

SEQ ID NO: 1041 - DNA, 20 bp, Arachis_hypogaea
cctccatgaatgtgaaccct

SEQ ID NO: 1042 - DNA, 20 bp, Arachis_hypogaea
aaacctagggttcacattca

SEQ ID NO: 1043 - DNA, 20 bp, Arachis_hypogaea
cctagggttcacattcatgg

SEQ ID NO: 1044 - DNA, 20 bp, Arachis_hypogaea
ttaatgaaccagagtgaaag

SEQ ID NO: 1045 - DNA, 20 bp, Arachis_hypogaea
cttgagatcctctttcactc

SEQ ID NO: 1046 - DNA, 20 bp, Arachis_hypogaea
acaaatacaaaagaaagtaa

SEQ ID NO: 1047 - DNA, 20 bp, Arachis_hypogaea
tcactaggaccaggaacatc

SEQ ID NO: 1048 - DNA, 20 bp, Arachis_hypogaea
atgacagatccagatgttcc

SEQ ID NO: 1049 - DNA, 20 bp, Arachis_hypogaea
aagtatgggtcactaggacc

SEQ ID NO: 1050 - DNA, 20 bp, Arachis_hypogaea
tctcttaagtatgggtcact

SEQ ID NO: 1051 - DNA, 20 bp, Arachis_hypogaea
gtaagtgttctcttaagtat

SEQ ID NO: 1052 - DNA, 20 bp, Arachis_hypogaea
tgtaagtgttctcttaagta

SEQ ID NO: 1053 - DNA, 20 bp, Arachis_hypogaea
acttaagagaacacttacac

SEQ ID NO: 1054 - DNA, 20 bp, Arachis_hypogaea
tcaactacttaattaaacga

SEQ ID NO: 1055 - DNA, 20 bp, Arachis_hypogaea
ttattagttgttatagtatt

SEQ ID NO: 1056 - DNA, 20 bp, Arachis_hypogaea
ttagttgtatatgtttgttt

SEQ ID NO: 1057 - DNA, 20 bp, Arachis_hypogaea
tagttgtatatgtttgtttt

SEQ ID NO: 1058 - DNA, 20 bp, Arachis_hypogaea
agttgtatatgtttgttttg

SEQ ID NO: 1059 - DNA, 20 bp, Arachis_hypogaea
aattcacaagaattaatttt

SEQ ID NO: 1060 - DNA, 20 bp, Arachis_hypogaea
aatatttaatttcttattat

SEQ ID NO: 1061 - DNA, 20 bp, Arachis_hypogaea
tttaatttcttattatagga

SEQ ID NO: 1062 - DNA, 20 bp, Arachis_hypogaea
aggatggtgactgatatccc

SEQ ID NO: 1063 - DNA, 20 bp, Arachis_hypogaea
atgttgcatccgttgtgcct

SEQ ID NO: 1064 - DNA, 20 bp, Arachis_hypogaea
aatgttgcatccgttgtgcc

SEQ ID NO: 1065 - DNA, 20 bp, Arachis_hypogaea
gactgatatcccaggcacaa

SEQ ID NO: 1066 - DNA, 20 bp, Arachis_hypogaea
ggcacaacggatgcaacatt

SEQ ID NO: 1067 - DNA, 20 bp, Arachis_hypogaea
aatactgtgaaagctaatta

SEQ ID NO: 1068 - DNA, 20 bp, Arachis_hypogaea
tgtaattaaaaaacacatac

SEQ ID NO: 1069 - DNA, 20 bp, Arachis_hypogaea
ttaaaaaacacatacaggaa

SEQ ID NO: 1070 - DNA, 20 bp, Arachis_hypogaea
aaaaacacatacaggaaagg

SEQ ID NO: 1071 - DNA, 20 bp, Arachis_hypogaea
tgtattcctatgtttggctt

SEQ ID NO: 1072 - DNA, 20 bp, Arachis_hypogaea
gagatgccaaagccaaacat

SEQ ID NO: 1073 - DNA, 20 bp, Arachis_hypogaea
aatctatgtattcctatgtt

SEQ ID NO: 1074 - DNA, 20 bp, Arachis_hypogaea
aggaatacatagatttgttt

SEQ ID NO: 1075 - DNA, 20 bp, Arachis_hypogaea
tggtgctttacaagcagaag

SEQ ID NO: 1076 - DNA, 20 bp, Arachis_hypogaea
tgctttacaagcagaagagg

SEQ ID NO: 1077 - DNA, 20 bp, Arachis_hypogaea
caagcagaagaggaggcaga

SEQ ID NO: 1078 - DNA, 20 bp, Arachis_hypogaea
tgaacaagattcccagttca

SEQ ID NO: 1079 - DNA, 20 bp, Arachis_hypogaea
ttgaagaggtcccttgaact

SEQ ID NO: 1080 - DNA, 20 bp, Arachis_hypogaea
gaacaagattcccagttcaa

SEQ ID NO: 1081 - DNA, 20 bp, Arachis_hypogaea
gttgaagaggtcccttgaac

SEQ ID NO: 1082 - DNA, 20 bp, Arachis_hypogaea
cgaatttccttgtgttgaag

SEQ ID NO: 1083 - DNA, 20 bp, Arachis_hypogaea
caagggacctcttcaacaca

SEQ ID NO: 1084 - DNA, 20 bp, Arachis_hypogaea
ttcgcagttgaaaatgatct

SEQ ID NO: 1085 - DNA, 20 bp, Arachis_hypogaea
aaatgatcttggcactcctg

SEQ ID NO: 1086 - DNA, 20 bp, Arachis_hypogaea
ttgaagaagacagcagccac

SEQ ID NO: 1087 - DNA, 20 bp, Arachis_hypogaea
tgatcagcgctttctacaag

SEQ ID NO: 1088 - DNA, 20 bp, Arachis_hypogaea
gtgtgcaactcatgtagtag

SEQ ID NO: 1089 - DNA, 20 bp, Arachis_hypogaea
atgtagtagtggcactatta

SEQ ID NO: 1090 - DNA, 20 bp, Arachis_hypogaea
tgttttacttatatataata

SEQ ID NO: 1091 - DNA, 20 bp, Arachis_hypogaea
tacttatatataatatggta

SEQ ID NO: 1092 - DNA, 20 bp, Arachis_hypogaea
acttatatataatatggtat

SEQ ID NO: 1093 - DNA, 20 bp, Arachis_hypogaea
agtcttaaacttattcttgg

SEQ ID NO: 1094 - DNA, 20 bp, Arachis_hypogaea
aacagtcttaaacttattct

SEQ ID NO: 1095 - DNA, 20 bp, Arachis_hypogaea
gaataagtttaagactgtta

SEQ ID NO: 1096 - DNA, 20 bp, Arachis_hypogaea
gtttaagactgttaaggtta

SEQ ID NO: 1097 - DNA, 20 bp, Arachis_hypogaea
tttaagactgttaaggttat

SEQ ID NO: 1098 - DNA, 1353 bp, Arachis hypogaea
tgcatacatagattggagcaattatatatctttgtattgaagaggtaactttaccttatattgaataagtaactaataagaaaaataattgatgatgatggcttcatcaaatgcattttcatcatcagatccacttactatagggagggtgattggagatgtagtcgatgatttcacagaaaccgtgaaaatgacagtcacttacaacaacaagcatgtctacaatggctttgagttcttcccttcttcaatttccgctaagcctagggttcacattcatggaggcgacttgagatcctctttcactctggttcattaatgattgatgatttcattacttaatatgttatatactctttatttcatgattatcatacctttactttttttttgtaattgtatttgtttgttccagattatgacagatccagatgttcctggtcctagtgacccatacttaagagaacacttacactggtaccttatatacatgcatgcactttaatttgatacgattaatttgttcaactacttaattaaacaatggatcttatttagttgttatagtattaggaattattaagtgatgtatagttgtatatgtttgttttaggggcacagaataacaaacaaattgtatacttttatttttaagacattcaagttagttaatgtgatttgcataaaataattagatattaattacttattataggatggtgactgatatcccagggacaacggatgcaacatttggtaagatattagttagtttactaattagttttcatagtattataatgaatgcatcctaagttcatgtaattaaaaacatatacaggaaaggaggtgataaagtatgagatgccaaagccaaacataggaatacatagatttgttttggtgctttacaagcagaagaggaggcaaacagtgagcaaggttcccaattcaagggacctcttcaacacaaggaaattcgtagttgaaaatgatcttggaactcctgtggctgctgtcttcttcaatgctcaaagagaaaccgcttgtaggaagcgctgatcagctgatctttaagacaaaaaaataatcttttatcttaaatgtgtgcaactcatgtagtagtggcactgttatggtgctttacttatatctaatatggtatgggatctaccaagaataagtttaagactgctaaggttatgggtatccattccctaaatagctggcctactattaactaactattctagtaagtaagttttatcagtttgcattgtagtttgctaattaagctttgtttaactttcattttctaattagttaagtttgagaccttaacttttctgtcta

SEQ ID NO: 1099 - protein, 179 aa, Arachis hypogaea
MMASSNAFSSSDPLTIGRVIGDVVDDFTETVKMTVTYNNKHVYNGYEFFPSSVSTKPRVHIHGGDLRSSFTLIMTDPDVPGPSDPYLREHLHWMVTDIPGTTDATFGKEVIKYEMPKPNIGIHRFVLVLYKQKRRQTVNKIPSSRDLFNTRKFAVENDLGTPVAAVFFNAQRETACRKR

SEQ ID NO: 1100 - DNA, 20 bp, Arachis_hypogaea
tatatatctttgtattgaag

SEQ ID NO: 1101 - DNA, 20 bp, Arachis_hypogaea
ttagttacttattcaatata

SEQ ID NO: 1102 - DNA, 20 bp, Arachis_hypogaea
agaaaaataattgatgatga

SEQ ID NO: 1103 - DNA, 20 bp, Arachis_hypogaea
atcaccctccctatagtaag

SEQ ID NO: 1104 - DNA, 20 bp, Arachis_hypogaea
tcatcagatccacttactat

SEQ ID NO: 1105 - DNA, 20 bp, Arachis_hypogaea
catcagatccacttactata

SEQ ID NO: 1106 - DNA, 20 bp, Arachis_hypogaea
cagatccacttactataggg

SEQ ID NO: 1107 - DNA, 20 bp, Arachis_hypogaea
agatccacttactataggga

SEQ ID NO: 1108 - DNA, 20 bp, Arachis_hypogaea
cttactatagggagggtgat

SEQ ID NO: 1109 - DNA, 20 bp, Arachis_hypogaea
gtaagtgactgtcattttca

SEQ ID NO: 1110 - DNA, 20 bp, Arachis_hypogaea
aacaacaagcatgtctacaa

SEQ ID NO: 1111 - DNA, 20 bp, Arachis_hypogaea
gcttagcggaaattgaagaa

SEQ ID NO: 1112 - DNA, 20 bp, Arachis_hypogaea
ggcttagcggaaattgaaga

SEQ ID NO: 1113 - DNA, 20 bp, Arachis_hypogaea
aatgtgaaccctaggcttag

SEQ ID NO: 1114 - DNA, 20 bp, Arachis_hypogaea
cttcaatttccgctaagcct

SEQ ID NO: 1115 - DNA, 20 bp, Arachis_hypogaea
ttcaatttccgctaagccta

SEQ ID NO: 1116 - DNA, 20 bp, Arachis_hypogaea
cctccatgaatgtgaaccct

SEQ ID NO: 1117 - DNA, 20 bp, Arachis_hypogaea
aagcctagggttcacattca

SEQ ID NO: 1118 - DNA, 20 bp, Arachis_hypogaea
cctagggttcacattcatgg

SEQ ID NO: 1119 - DNA, 20 bp, Arachis_hypogaea
ttaatgaaccagagtgaaag

SEQ ID NO: 1120 - DNA, 20 bp, Arachis_hypogaea
cttgagatcctctttcactc

SEQ ID NO: 1121 - DNA, 20 bp, Arachis_hypogaea
caattacaaaaaaaaagtaa

SEQ ID NO: 1122 - DNA, 20 bp, Arachis_hypogaea
catctggatctgtcataatc

SEQ ID NO: 1123 - DNA, 20 bp, Arachis_hypogaea
tcactaggaccaggaacatc

SEQ ID NO: 1124 - DNA, 20 bp, Arachis_hypogaea
atgacagatccagatgttcc

SEQ ID NO: 1125 - DNA, 20 bp, Arachis_hypogaea
aagtatgggtcactaggacc

SEQ ID NO: 1126 - DNA, 20 bp, Arachis_hypogaea
tctcttaagtatgggtcact

SEQ ID NO: 1127 - DNA, 20 bp, Arachis_hypogaea
gtaagtgttctcttaagtat

SEQ ID NO: 1128 - DNA, 20 bp, Arachis_hypogaea
tgtaagtgttctcttaagta

SEQ ID NO: 1129 - DNA, 20 bp, Arachis_hypogaea
acttaagagaacacttacac

SEQ ID NO: 1130 - DNA, 20 bp, Arachis_hypogaea
aaagtgcatgcatgtatata

SEQ ID NO: 1131 - DNA, 20 bp, Arachis_hypogaea
tcaactacttaattaaacaa

SEQ ID NO: 1132 - DNA, 20 bp, Arachis_hypogaea
tatttagttgttatagtatt

SEQ ID NO: 1133 - DNA, 20 bp, Arachis_hypogaea
tagttgtatatgtttgtttt

SEQ ID NO: 1134 - DNA, 20 bp, Arachis_hypogaea
agttgtatatgtttgtttta

SEQ ID NO: 1135 - DNA, 20 bp, Arachis_hypogaea
gttgtatatgtttgttttag

SEQ ID NO: 1136 - DNA, 20 bp, Arachis_hypogaea
agatattaattacttattat

SEQ ID NO: 1137 - DNA, 20 bp, Arachis_hypogaea
attaattacttattatagga

SEQ ID NO: 1138 - DNA, 20 bp, Arachis_hypogaea
aggatggtgactgatatccc

SEQ ID NO: 1139 - DNA, 20 bp, Arachis_hypogaea
ggatggtgactgatatccca

SEQ ID NO: 1140 - DNA, 20 bp, Arachis_hypogaea
atgttgcatccgttgtccct

SEQ ID NO: 1141 - DNA, 20 bp, Arachis_hypogaea
aatgttgcatccgttgtccc

SEQ ID NO: 1142 - DNA, 20 bp, Arachis_hypogaea
gactgatatcccagggacaa

SEQ ID NO: 1143 - DNA, 20 bp, Arachis_hypogaea
gggacaacggatgcaacatt

SEQ ID NO: 1144 - DNA, 20 bp, Arachis_hypogaea
gtttttaattacatgaactt

SEQ ID NO: 1145 - DNA, 20 bp, Arachis_hypogaea
atgtaattaaaaacatatac

SEQ ID NO: 1146 - DNA, 20 bp, Arachis_hypogaea
attaaaaacatatacaggaa

SEQ ID NO: 1147 - DNA, 20 bp, Arachis_hypogaea
aaaaacatatacaggaaagg

SEQ ID NO: 1148 - DNA, 20 bp, Arachis_hypogaea
tgtattcctatgtttggctt

SEQ ID NO: 1149 - DNA, 20 bp, Arachis_hypogaea
gagatgccaaagccaaacat

SEQ ID NO: 1150 - DNA, 20 bp, Arachis_hypogaea
aatctatgtattcctatgtt

SEQ ID NO: 1151 - DNA, 20 bp, Arachis_hypogaea
aggaatacatagatttgttt

SEQ ID NO: 1152 - DNA, 20 bp, Arachis_hypogaea
tggtgctttacaagcagaag

SEQ ID NO: 1153 - DNA, 20 bp, Arachis_hypogaea
tgctttacaagcagaagagg

SEQ ID NO: 1154 - DNA, 20 bp, Arachis_hypogaea
gaggaggcaaacagtgagca

SEQ ID NO: 1155 - DNA, 20 bp, Arachis_hypogaea
tgagcaaggttcccaattca

SEQ ID NO: 1156 - DNA, 20 bp, Arachis_hypogaea
ttgaagaggtcccttgaatt

SEQ ID NO: 1157 - DNA, 20 bp, Arachis_hypogaea
gagcaaggttcccaattcaa

SEQ ID NO: 1158 - DNA, 20 bp, Arachis_hypogaea
gttgaagaggtcccttgaat

SEQ ID NO: 1159 - DNA, 20 bp, Arachis_hypogaea
cgaatttccttgtgttgaag

SEQ ID NO: 1160 - DNA, 20 bp, Arachis_hypogaea
caagggacctcttcaacaca

SEQ ID NO: 1161 - DNA, 20 bp, Arachis_hypogaea
ttcgtagttgaaaatgatct

SEQ ID NO: 1162 - DNA, 20 bp, Arachis_hypogaea
aaatgatcttggaactcctg

SEQ ID NO: 1163 - DNA, 20 bp, Arachis_hypogaea
ttgaagaagacagcagccac

SEQ ID NO: 1164 - DNA, 20 bp, Arachis_hypogaea
ctcaaagagaaaccgcttgt

SEQ ID NO: 1165 - DNA, 20 bp, Arachis_hypogaea
tgatcagcgcttcctacaag

SEQ ID NO: 1166 - DNA, 20 bp, Arachis_hypogaea
gtgtgcaactcatgtagtag

SEQ ID NO: 1167 - DNA, 20 bp, Arachis_hypogaea
atgtagtagtggcactgtta

SEQ ID NO: 1168 - DNA, 20 bp, Arachis_hypogaea
tgctttacttatatctaata

SEQ ID NO: 1169 - DNA, 20 bp, Arachis_hypogaea
tacttatatctaatatggta

SEQ ID NO: 1170 - DNA, 20 bp, Arachis_hypogaea
acttatatctaatatggtat

SEQ ID NO: 1171 - DNA, 20 bp, Arachis_hypogaea
agcagtcttaaacttattct

SEQ ID NO: 1172 - DNA, 20 bp, Arachis_hypogaea
gaataagtttaagactgcta

SEQ ID NO: 1173 - DNA, 20 bp, Arachis_hypogaea
gtttaagactgctaaggtta

SEQ ID NO: 1174 - DNA, 20 bp, Arachis_hypogaea
tttaagactgctaaggttat

SEQ ID NO: 1175 - DNA, 20 bp, Arachis_hypogaea
taggccagctatttagggaa

SEQ ID NO: 1176 - DNA, 20 bp, Arachis_hypogaea
aatagtaggccagctattta

SEQ ID NO: 1177 - DNA, 20 bp, Arachis_hypogaea
taatagtaggccagctattt

SEQ ID NO: 1178 - DNA, 20 bp, Arachis_hypogaea
gtatccattccctaaatagc

SEQ ID NO: 1179 - DNA, 20 bp, Arachis_hypogaea
tagaatagttagttaatagt

SEQ ID NO: 1180 - DNA, 1525 bp, Arachis hypogaea
aagctaagctagtaattgtatttgtatctctcactctttattaatttcctctatcttcttcttatttgcttgttaataagaattatattattctcctaatggcaagaataatgtcatcagagcctttaattgtagggagagtggtaggagatgttcttgatccattcaatgcaagcataaggatgagtgttagttatgggaacaggcaagtgtacaatggccatgagttcttcccctccacagtcaccttcaagcccaaggttgagattggtggaactgaattgaggcccttctttacactggttggtacctctatatatatatatactcctctcatctcttcttcttttgttcctttttctctttataggtttaaaaatgggaaaatctttggcagtttggctgttgttattatctttatataataatagtaatagtagctggctctattataatagctggtggctacttctacgagaatgtcttcgtgtaaagacaatatatgaattattagatgatgcagttaaacagttcataatatcatctacgttttcatagaagtagccaacactaatacggtcagtagtattatgatacatatatatacgtagaatgtaaatgtctctcttttgggttgcaggtcatgactgatccggatgttcctggccctagtgatccttatctcagagagcacttgcattggtaaaacttaattaaatttttactacatgaaaattaattagggtaacaactaaaaccaaaataaaacagctgtttattacaaacacataatgcatcatttggtcatatgaaagagacacacatattatcacgattcataggcatgcattaatttcacatagctagaggtgtatctagatctctaagagtcaagcatattaaaaaaattgaaatatatatctgcaggatagttacagacattccaggcacaacagatgccacatttggtaggtctctcattcctcttttgatatctatggacactacaagactgagaaataataataatgatgttgatgtatgtttaattatttgttttagggaaagagttggtgagctacgagatgccaaagcctaacattgggatccataggtatgtttttgtgctcttcaagcaaaaacgaaggcagtgtgttagcaccattccttcttgcagggatcacttcaacacccgcactttcgccgccgataacgacctcggcctccccgtcgctgccgtctacttcaatgctcagagggaaaccgctgccaggaggcgttaattaattaattaagaagtagctagagatccaagtgtgtctgaatgaaaaataatttacttgtgttgcagaaaaaagggaatcatttcttttttgactatcatgtatgtatgtatgtatgtatgtatgtatgtatgtagaagggttactttttcattatcacatatatgctgtgtgttttaatttccatggtatatacgccatgtcgtttcatctaatcataataattactactttaggatttg

SEQ ID NO: 1181 - protein, 175 aa, Arachis hypogaea
MARIMSSEPLIVGRVVGDVLDPFNASIRMSVSYGNRQVYNGHEFFPSTVTFKPKVEIGGTELRPFFTLVMTDPDVPGPSDPYLREHLHWIVTDIPGTTDATFGKELVSYEMPKPNIGIHRYVFVLFKQKRRQCVSTIPSCRDHFNTRTFAADNDLGLPVAAVYFNAQRETAARRR

SEQ ID NO: 1182 - DNA, 20 bp, Arachis_hypogaea
agcaaataagaagaagatag

SEQ ID NO: 1183 - DNA, 20 bp, Arachis_hypogaea
gaattatattattctcctaa

SEQ ID NO: 1184 - DNA, 20 bp, Arachis_hypogaea
atgacattattcttgccatt

SEQ ID NO: 1185 - DNA, 20 bp, Arachis_hypogaea
accactctccctacaattaa

SEQ ID NO: 1186 - DNA, 20 bp, Arachis_hypogaea
tcatcagagcctttaattgt

SEQ ID NO: 1187 - DNA, 20 bp, Arachis_hypogaea
catcagagcctttaattgta

SEQ ID NO: 1188 - DNA, 20 bp, Arachis_hypogaea
gcctttaattgtagggagag

SEQ ID NO: 1189 - DNA, 20 bp, Arachis_hypogaea
ttaattgtagggagagtggt

SEQ ID NO: 1190 - DNA, 20 bp, Arachis_hypogaea
atccttatgcttgcattgaa

SEQ ID NO: 1191 - DNA, 20 bp, Arachis_hypogaea
atccattcaatgcaagcata

SEQ ID NO: 1192 - DNA, 20 bp, Arachis_hypogaea
ataaggatgagtgttagtta

SEQ ID NO: 1193 - DNA, 20 bp, Arachis_hypogaea
taaggatgagtgttagttat

SEQ ID NO: 1194 - DNA, 20 bp, Arachis_hypogaea
tgagtgttagttatgggaac

SEQ ID NO: 1195 - DNA, 20 bp, Arachis_hypogaea
gggaacaggcaagtgtacaa

SEQ ID NO: 1196 - DNA, 20 bp, Arachis_hypogaea
ctgtggaggggaagaactca

SEQ ID NO: 1197 - DNA, 20 bp, Arachis_hypogaea
gcttgaaggtgactgtggag

SEQ ID NO: 1198 - DNA, 20 bp, Arachis_hypogaea
ggcttgaaggtgactgtgga

SEQ ID NO: 1199 - DNA, 20 bp, Arachis_hypogaea
gggcttgaaggtgactgtgg

SEQ ID NO: 1200 - DNA, 20 bp, Arachis_hypogaea
cttgggcttgaaggtgactg

SEQ ID NO: 1201 - DNA, 20 bp, Arachis_hypogaea
aatctcaaccttgggcttga

SEQ ID NO: 1202 - DNA, 20 bp, Arachis_hypogaea
cacagtcaccttcaagccca

SEQ ID NO: 1203 - DNA, 20 bp, Arachis_hypogaea
gttccaccaatctcaacctt

SEQ ID NO: 1204 - DNA, 20 bp, Arachis_hypogaea
agttccaccaatctcaacct

SEQ ID NO: 1205 - DNA, 20 bp, Arachis_hypogaea
ttcaagcccaaggttgagat

SEQ ID NO: 1206 - DNA, 20 bp, Arachis_hypogaea
aagcccaaggttgagattgg

SEQ ID NO: 1207 - DNA, 20 bp, Arachis_hypogaea
agattggtggaactgaattg

SEQ ID NO: 1208 - DNA, 20 bp, Arachis_hypogaea
gtaccaaccagtgtaaagaa

SEQ ID NO: 1209 - DNA, 20 bp, Arachis_hypogaea
ggtaccaaccagtgtaaaga

SEQ ID NO: 1210 - DNA, 20 bp, Arachis_hypogaea
attgaggcccttctttacac

SEQ ID NO: 1211 - DNA, 20 bp, Arachis_hypogaea
aggcccttctttacactggt

SEQ ID NO: 1212 - DNA, 20 bp, Arachis_hypogaea
aggagtatatatatatatag

SEQ ID NO: 1213 - DNA, 20 bp, Arachis_hypogaea
acaaaagaagaagagatgag

SEQ ID NO: 1214 - DNA, 20 bp, Arachis_hypogaea
ttaaacctataaagagaaaa

SEQ ID NO: 1215 - DNA, 20 bp, Arachis_hypogaea
ttgttcctttttctctttat

SEQ ID NO: 1216 - DNA, 20 bp, Arachis_hypogaea
tctctttataggtttaaaaa

SEQ ID NO: 1217 - DNA, 20 bp, Arachis_hypogaea
ctctttataggtttaaaaat

SEQ ID NO: 1218 - DNA, 20 bp, Arachis_hypogaea
tttaaaaatgggaaaatctt

SEQ ID NO: 1219 - DNA, 20 bp, Arachis_hypogaea
tgggaaaatctttggcagtt

SEQ ID NO: 1220 - DNA, 20 bp, Arachis_hypogaea
ataataatagtaatagtagc

SEQ ID NO: 1221 - DNA, 20 bp, Arachis_hypogaea
gctggctctattataatagc

SEQ ID NO: 1222 - DNA, 20 bp, Arachis_hypogaea
ggctctattataatagctgg

SEQ ID NO: 1223 - DNA, 20 bp, Arachis_hypogaea
actactgaccgtattagtgt

SEQ ID NO: 1224 - DNA, 20 bp, Arachis_hypogaea
agaagtagccaacactaata

SEQ ID NO: 1225 - DNA, 20 bp, Arachis_hypogaea
gaatgtaaatgtctctcttt

SEQ ID NO: 1226 - DNA, 20 bp, Arachis_hypogaea
aatgtaaatgtctctctttt

SEQ ID NO: 1227 - DNA, 20 bp, Arachis_hypogaea
atgtctctcttttgggttgc

SEQ ID NO: 1228 - DNA, 20 bp, Arachis_hypogaea
gttgcaggtcatgactgatc

SEQ ID NO: 1229 - DNA, 20 bp, Arachis_hypogaea
tcactagggccaggaacatc

SEQ ID NO: 1230 - DNA, 20 bp, Arachis_hypogaea
atgactgatccggatgttcc

SEQ ID NO: 1231 - DNA, 20 bp, Arachis_hypogaea
agataaggatcactagggcc

SEQ ID NO: 1232 - DNA, 20 bp, Arachis_hypogaea
ctctgagataaggatcacta

SEQ ID NO: 1233 - DNA, 20 bp, Arachis_hypogaea
tctctgagataaggatcact

SEQ ID NO: 1234 - DNA, 20 bp, Arachis_hypogaea
tgcaagtgctctctgagata

SEQ ID NO: 1235 - DNA, 20 bp, Arachis_hypogaea
atctcagagagcacttgcat

SEQ ID NO: 1236 - DNA, 20 bp, Arachis_hypogaea
ttactacatgaaaattaatt

SEQ ID NO: 1237 - DNA, 20 bp, Arachis_hypogaea
tactacatgaaaattaatta

SEQ ID NO: 1238 - DNA, 20 bp, Arachis_hypogaea
aataaacagctgttttattt

SEQ ID NO: 1239 - DNA, 20 bp, Arachis_hypogaea
caaacacataatgcatcatt

SEQ ID NO: 1240 - DNA, 20 bp, Arachis_hypogaea
cacatattatcacgattcat

SEQ ID NO: 1241 - DNA, 20 bp, Arachis_hypogaea
cattaatttcacatagctag

SEQ ID NO: 1242 - DNA, 20 bp, Arachis_hypogaea
aaattgaaatatatatctgc

SEQ ID NO: 1243 - DNA, 20 bp, Arachis_hypogaea
aggatagttacagacattcc

SEQ ID NO: 1244 - DNA, 20 bp, Arachis_hypogaea
aatgtggcatctgttgtgcc

SEQ ID NO: 1245 - DNA, 20 bp, Arachis_hypogaea
ggcacaacagatgccacatt

SEQ ID NO: 1246 - DNA, 20 bp, Arachis_hypogaea
aatgagagacctaccaaatg

SEQ ID NO: 1247 - DNA, 20 bp, Arachis_hypogaea
caacagatgccacatttggt

SEQ ID NO: 1248 - DNA, 20 bp, Arachis_hypogaea
gtgtccatagatatcaaaag

SEQ ID NO: 1249 - DNA, 20 bp, Arachis_hypogaea
cattcctcttttgatatcta

SEQ ID NO: 1250 - DNA, 20 bp, Arachis_hypogaea
tatgtttaattatttgtttt

SEQ ID NO: 1251 - DNA, 20 bp, Arachis_hypogaea
atgtttaattatttgtttta

SEQ ID NO: 1252 - DNA, 20 bp, Arachis_hypogaea
atttgttttagggaaagagt

SEQ ID NO: 1253 - DNA, 20 bp, Arachis_hypogaea
tggatcccaatgttaggctt

SEQ ID NO: 1254 - DNA, 20 bp, Arachis_hypogaea
gagatgccaaagcctaacat

SEQ ID NO: 1255 - DNA, 20 bp, Arachis_hypogaea
agatgccaaagcctaacatt

SEQ ID NO: 1256 - DNA, 20 bp, Arachis_hypogaea
tacctatggatcccaatgtt

SEQ ID NO: 1257 - DNA, 20 bp, Arachis_hypogaea
agcctaacattgggatccat

SEQ ID NO: 1258 - DNA, 20 bp, Arachis_hypogaea
agagcacaaaaacataccta

SEQ ID NO: 1259 - DNA, 20 bp, Arachis_hypogaea
tgctcttcaagcaaaaacga

SEQ ID NO: 1260 - DNA, 20 bp, Arachis_hypogaea
gtgatccctgcaagaaggaa

SEQ ID NO: 1261 - DNA, 20 bp, Arachis_hypogaea
ttagcaccattccttcttgc

SEQ ID NO: 1262 - DNA, 20 bp, Arachis_hypogaea
ttgaagtgatccctgcaaga

SEQ ID NO: 1263 - DNA, 20 bp, Arachis_hypogaea
tagcaccattccttcttgca

SEQ ID NO: 1264 - DNA, 20 bp, Arachis_hypogaea
gttatcggcggcgaaagtgc

SEQ ID NO: 1265 - DNA, 20 bp, Arachis_hypogaea
cgttatcggcggcgaaagtg

SEQ ID NO: 1266 - DNA, 20 bp, Arachis_hypogaea
gaggccgaggtcgttatcgg

SEQ ID NO: 1267 - DNA, 20 bp, Arachis_hypogaea
ggggaggccgaggtcgttat

SEQ ID NO: 1268 - DNA, 20 bp, Arachis_hypogaea
ttcgccgccgataacgacct

SEQ ID NO: 1269 - DNA, 20 bp, Arachis_hypogaea
cggcagcgacggggaggccg

SEQ ID NO: 1270 - DNA, 20 bp, Arachis_hypogaea
agtagacggcagcgacgggg

SEQ ID NO: 1271 - DNA, 20 bp, Arachis_hypogaea
tgaagtagacggcagcgacg

SEQ ID NO: 1272 - DNA, 20 bp, Arachis_hypogaea
ttgaagtagacggcagcgac

SEQ ID NO: 1273 - DNA, 20 bp, Arachis_hypogaea
attgaagtagacggcagcga

SEQ ID NO: 1274 - DNA, 20 bp, Arachis_hypogaea
cctctgagcattgaagtaga

SEQ ID NO: 1275 - DNA, 20 bp, Arachis_hypogaea
ccgtctacttcaatgctcag

SEQ ID NO: 1276 - DNA, 20 bp, Arachis_hypogaea
cgtctacttcaatgctcaga

SEQ ID NO: 1277 - DNA, 20 bp, Arachis_hypogaea
ctcagagggaaaccgctgcc

SEQ ID NO: 1278 - DNA, 20 bp, Arachis_hypogaea
taattaacgcctcctggcag

SEQ ID NO: 1279 - DNA, 20 bp, Arachis_hypogaea
agagggaaaccgctgccagg

SEQ ID NO: 1280 - DNA, 20 bp, Arachis_hypogaea
attaattaattaacgcctcc

SEQ ID NO: 1281 - DNA, 20 bp, Arachis_hypogaea
atttttcattcagacacact

SEQ ID NO: 1282 - DNA, 20 bp, Arachis_hypogaea
ttacttgtgttgcagaaaaa

SEQ ID NO: 1283 - DNA, 20 bp, Arachis_hypogaea
tacttgtgttgcagaaaaaa

SEQ ID NO: 1284 - DNA, 20 bp, Arachis_hypogaea
tatgtatgtatgtatgtaga

SEQ ID NO: 1285 - DNA, 20 bp, Arachis_hypogaea
atgtatgtatgtatgtagaa

SEQ ID NO: 1286 - DNA, 20 bp, Arachis_hypogaea
gctgtgtgttttaatttcca

SEQ ID NO: 1287 - DNA, 20 bp, Arachis_hypogaea
acgacatggcgtatatacca

SEQ ID NO: 1288 - DNA, 20 bp, Arachis_hypogaea
tatgattagatgaaacgaca

SEQ ID NO: 1289 - DNA, 20 bp, Arachis_hypogaea
atcataataattactacttt

SEQ ID NO: 1290 - DNA, 1751 bp, Arachis hypogaea
gaggagtggtgggagcatatgatttgtttcagccctcacatagacccaattattagaataaacaaatgatatcttagtgtgttactacacatgtattcacaaaaatgaatattaaaaacacatagctggaagattctcttgatagagacattataaatatggaactcaagtgtacttactttcctcaagctaagctagtaattgtatttgtatctctcactctttattaatttcctctatcttcttcttatttgcttgttaataagaattatattattctcctaatggcaagaataatgtcatcagagcctttaattgtagggagagtggtaggagatgttcttgatccattcaatgcaagcataaggatgagtgttagttatgggaacaggcaagtgtacaatggccatgagttcttcccctccacagtcaccttcaagcccaaggttgagataggtggaactgaattgaggcccttctttacactggttggtacctctctctctctctctctctatatatatatatatatatatatatactcctctcatctcttcttcttttgttcctttttctctttataggtttaaaaatgggaaaacctttggcagtttggctgttgttatctttatataataatagtaatagtagctcgctctattataatagctggtggttacttctacgagaatgtcttcgtgtaatgacaatatataaattattagatgattcagttaaacaagtcaaattatttaaacggttcataatatcatctttacacgaattcgttttcgtagaagtagccaacactaacacgatcagtattatgatacatatatataaatacatagaatgtaaatgtctctcttttgggttgcaggtcatgactgatccggatgttcctggccctagtgatccttatctcagagagcacttgcattggtaaaacttaattaaatttttactacatgaaaattaatcaggttaacaactaaaaccaaaataaaacagctgtttattacaaacacataatgcatcatttggtcatatgaaagagacacacacatattatcacgactcataggcatgcattaatttcacacagctagaggtgtatctagatctctaagagtcaagcaaattaaaaaaaatgaaatatttatctgcaggatagtgacagacattccaggcacaacagatgccacatttggtaggtctctcattcctcttttgatatctatggatactacaagactgagaaataataataatgttgttgatgtatgtttaattatttgttttagggaaagagttggtgagctacgagatgccaaagcctaacattgggatccataggtatgtttttgtgctcttcaagcaaaaacgaaggcagtgtgttaccaccattccttcttgcagggatcacttcaacacccgcactttcgccgccgacaacgacctcggcctccctgtcgctgccgtctacttcaatgctcagagggaaaccgctgccaggaggcgttaattaattaattaagaagtagctagagatcccagtgtgggtgaatgaaaaataatttacttgtattgcagaaaaaagggaatcatttcttttttgactatcatgtatgtatgtatgtatgtagaagggttactttttcgttatcacatatatgctgtgtgttttaatttccatggtatatacgccatgtcgtttcatctaatcataataattactactttaggatttg

SEQ ID NO: 1291 - protein, 175 aa, Arachis hypogaea
MARIMSSEPLIVGRVVGDVLDPFNASIRMSVSYGNRQVYNGHEFFPSTVTFKPKVEIGGTELRPFFTLVMTDPDVPGPSDPYLREHLHWIVTDIPGTTDATFGKELVSYEMPKPNIGIHRYVFVLFKQKRRQCVTTIPSCRDHFNTRTFAADNDLGLPVAAVYFNAQRETAARRR

SEQ ID NO: 1292 - DNA, 20 bp, Arachis_hypogaea
taataattgggtctatgtga

SEQ ID NO: 1293 - DNA, 20 bp, Arachis_hypogaea
ctaataattgggtctatgtg

SEQ ID NO: 1294 - DNA, 20 bp, Arachis_hypogaea
atttgtttattctaataatt

SEQ ID NO: 1295 - DNA, 20 bp, Arachis_hypogaea
catttgtttattctaataat

SEQ ID NO: 1296 - DNA, 20 bp, Arachis_hypogaea
gaatattaaaaacacatagc

SEQ ID NO: 1297 - DNA, 20 bp, Arachis_hypogaea
tgatagagacattataaata

SEQ ID NO: 1298 - DNA, 20 bp, Arachis_hypogaea
acaattactagcttagcttg

SEQ ID NO: 1299 - DNA, 20 bp, Arachis_hypogaea
agcaaataagaagaagatag

SEQ ID NO: 1300 - DNA, 20 bp, Arachis_hypogaea
gaattatattattctcctaa

SEQ ID NO: 1301 - DNA, 20 bp, Arachis_hypogaea
atgacattattcttgccatt

SEQ ID NO: 1302 - DNA, 20 bp, Arachis_hypogaea
accactctccctacaattaa

SEQ ID NO: 1303 - DNA, 20 bp, Arachis_hypogaea
tcatcagagcctttaattgt

SEQ ID NO: 1304 - DNA, 20 bp, Arachis_hypogaea
catcagagcctttaattgta

SEQ ID NO: 1305 - DNA, 20 bp, Arachis_hypogaea
gcctttaattgtagggagag

SEQ ID NO: 1306 - DNA, 20 bp, Arachis_hypogaea
ttaattgtagggagagtggt

SEQ ID NO: 1307 - DNA, 20 bp, Arachis_hypogaea
atccttatgcttgcattgaa

SEQ ID NO: 1308 - DNA, 20 bp, Arachis_hypogaea
atccattcaatgcaagcata

SEQ ID NO: 1309 - DNA, 20 bp, Arachis_hypogaea
ataaggatgagtgttagtta

SEQ ID NO: 1310 - DNA, 20 bp, Arachis_hypogaea
taaggatgagtgttagttat

SEQ ID NO: 1311 - DNA, 20 bp, Arachis_hypogaea
tgagtgttagttatgggaac

SEQ ID NO: 1312 - DNA, 20 bp, Arachis_hypogaea
gggaacaggcaagtgtacaa

SEQ ID NO: 1313 - DNA, 20 bp, Arachis_hypogaea
ctgtggaggggaagaactca

SEQ ID NO: 1314 - DNA, 20 bp, Arachis_hypogaea
gcttgaaggtgactgtggag

SEQ ID NO: 1315 - DNA, 20 bp, Arachis_hypogaea
ggcttgaaggtgactgtgga

SEQ ID NO: 1316 - DNA, 20 bp, Arachis_hypogaea
gggcttgaaggtgactgtgg

SEQ ID NO: 1317 - DNA, 20 bp, Arachis_hypogaea
cttgggcttgaaggtgactg

SEQ ID NO: 1318 - DNA, 20 bp, Arachis_hypogaea
tatctcaaccttgggcttga

SEQ ID NO: 1319 - DNA, 20 bp, Arachis_hypogaea
cacagtcaccttcaagccca

SEQ ID NO: 1320 - DNA, 20 bp, Arachis_hypogaea
gttccacctatctcaacctt

SEQ ID NO: 1321 - DNA, 20 bp, Arachis_hypogaea
agttccacctatctcaacct

SEQ ID NO: 1322 - DNA, 20 bp, Arachis_hypogaea
ttcaagcccaaggttgagat

SEQ ID NO: 1323 - DNA, 20 bp, Arachis_hypogaea
aagcccaaggttgagatagg

SEQ ID NO: 1324 - DNA, 20 bp, Arachis_hypogaea
agataggtggaactgaattg

SEQ ID NO: 1325 - DNA, 20 bp, Arachis_hypogaea
gtaccaaccagtgtaaagaa

SEQ ID NO: 1326 - DNA, 20 bp, Arachis_hypogaea
ggtaccaaccagtgtaaaga

SEQ ID NO: 1327 - DNA, 20 bp, Arachis_hypogaea
attgaggcccttctttacac

SEQ ID NO: 1328 - DNA, 20 bp, Arachis_hypogaea
aggcccttctttacactggt

SEQ ID NO: 1329 - DNA, 20 bp, Arachis_hypogaea
atagagagagagagagagag

SEQ ID NO: 1330 - DNA, 20 bp, Arachis_hypogaea
acaaaagaagaagagatgag

SEQ ID NO: 1331 - DNA, 20 bp, Arachis_hypogaea
ttaaacctataaagagaaaa

SEQ ID NO: 1332 - DNA, 20 bp, Arachis_hypogaea
ttgttcctttttctctttat

SEQ ID NO: 1333 - DNA, 20 bp, Arachis_hypogaea
tctctttataggtttaaaaa

SEQ ID NO: 1334 - DNA, 20 bp, Arachis_hypogaea
ctctttataggtttaaaaat

SEQ ID NO: 1335 - DNA, 20 bp, Arachis_hypogaea
tttaaaaatgggaaaacctt

SEQ ID NO: 1336 - DNA, 20 bp, Arachis_hypogaea
aacaacagccaaactgccaa

SEQ ID NO: 1337 - DNA, 20 bp, Arachis_hypogaea
tgggaaaacctttggcagtt

SEQ ID NO: 1338 - DNA, 20 bp, Arachis_hypogaea
gctcgctctattataatagc

SEQ ID NO: 1339 - DNA, 20 bp, Arachis_hypogaea
cgctctattataatagctgg

SEQ ID NO: 1340 - DNA, 20 bp, Arachis_hypogaea
aacaagtcaaattatttaaa

SEQ ID NO: 1341 - DNA, 20 bp, Arachis_hypogaea
aatactgatcgtgttagtgt

SEQ ID NO: 1342 - DNA, 20 bp, Arachis_hypogaea
gaatgtaaatgtctctcttt

SEQ ID NO: 1343 - DNA, 20 bp, Arachis_hypogaea
aatgtaaatgtctctctttt

SEQ ID NO: 1344 - DNA, 20 bp, Arachis_hypogaea
atgtctctcttttgggttgc

SEQ ID NO: 1345 - DNA, 20 bp, Arachis_hypogaea
gttgcaggtcatgactgatc

SEQ ID NO: 1346 - DNA, 20 bp, Arachis_hypogaea
tcactagggccaggaacatc

SEQ ID NO: 1347 - DNA, 20 bp, Arachis_hypogaea
atgactgatccggatgttcc

SEQ ID NO: 1348 - DNA, 20 bp, Arachis_hypogaea
agataaggatcactagggcc

SEQ ID NO: 1349 - DNA, 20 bp, Arachis_hypogaea
ctctgagataaggatcacta

SEQ ID NO: 1350 - DNA, 20 bp, Arachis_hypogaea
tctctgagataaggatcact

SEQ ID NO: 1351 - DNA, 20 bp, Arachis_hypogaea
tgcaagtgctctctgagata

SEQ ID NO: 1352 - DNA, 20 bp, Arachis_hypogaea
atctcagagagcacttgcat

SEQ ID NO: 1353 - DNA, 20 bp, Arachis_hypogaea
ttactacatgaaaattaatc

SEQ ID NO: 1354 - DNA, 20 bp, Arachis_hypogaea
aataaacagctgttttattt

SEQ ID NO: 1355 - DNA, 20 bp, Arachis_hypogaea
caaacacataatgcatcatt

SEQ ID NO: 1356 - DNA, 20 bp, Arachis_hypogaea
cacatattatcacgactcat

SEQ ID NO: 1357 - DNA, 20 bp, Arachis_hypogaea
cattaatttcacacagctag

SEQ ID NO: 1358 - DNA, 20 bp, Arachis_hypogaea
aaaatgaaatatttatctgc

SEQ ID NO: 1359 - DNA, 20 bp, Arachis_hypogaea
aggatagtgacagacattcc

SEQ ID NO: 1360 - DNA, 20 bp, Arachis_hypogaea
aatgtggcatctgttgtgcc

SEQ ID NO: 1361 - DNA, 20 bp, Arachis_hypogaea
ggcacaacagatgccacatt

SEQ ID NO: 1362 - DNA, 20 bp, Arachis_hypogaea
aatgagagacctaccaaatg

SEQ ID NO: 1363 - DNA, 20 bp, Arachis_hypogaea
caacagatgccacatttggt

SEQ ID NO: 1364 - DNA, 20 bp, Arachis_hypogaea
gtatccatagatatcaaaag

SEQ ID NO: 1365 - DNA, 20 bp, Arachis_hypogaea
cattcctcttttgatatcta

SEQ ID NO: 1366 - DNA, 20 bp, Arachis_hypogaea
tatgtttaattatttgtttt

SEQ ID NO: 1367 - DNA, 20 bp, Arachis_hypogaea
atgtttaattatttgtttta

SEQ ID NO: 1368 - DNA, 20 bp, Arachis_hypogaea
atttgttttagggaaagagt

SEQ ID NO: 1369 - DNA, 20 bp, Arachis_hypogaea
tggatcccaatgttaggctt

SEQ ID NO: 1370 - DNA, 20 bp, Arachis_hypogaea
gagatgccaaagcctaacat

SEQ ID NO: 1371 - DNA, 20 bp, Arachis_hypogaea
agatgccaaagcctaacatt

SEQ ID NO: 1372 - DNA, 20 bp, Arachis_hypogaea
tacctatggatcccaatgtt

SEQ ID NO: 1373 - DNA, 20 bp, Arachis_hypogaea
agcctaacattgggatccat

SEQ ID NO: 1374 - DNA, 20 bp, Arachis_hypogaea
agagcacaaaaacataccta

SEQ ID NO: 1375 - DNA, 20 bp, Arachis_hypogaea
tgctcttcaagcaaaaacga

SEQ ID NO: 1376 - DNA, 20 bp, Arachis_hypogaea
atccctgcaagaaggaatgg

SEQ ID NO: 1377 - DNA, 20 bp, Arachis_hypogaea
gtgatccctgcaagaaggaa

SEQ ID NO: 1378 - DNA, 20 bp, Arachis_hypogaea
ttaccaccattccttcttgc

SEQ ID NO: 1379 - DNA, 20 bp, Arachis_hypogaea
ttgaagtgatccctgcaaga

SEQ ID NO: 1380 - DNA, 20 bp, Arachis_hypogaea
taccaccattccttcttgca

SEQ ID NO: 1381 - DNA, 20 bp, Arachis_hypogaea
gttgtcggcggcgaaagtgc

SEQ ID NO: 1382 - DNA, 20 bp, Arachis_hypogaea
cgttgtcggcggcgaaagtg

SEQ ID NO: 1383 - DNA, 20 bp, Arachis_hypogaea
gaggccgaggtcgttgtcgg

SEQ ID NO: 1384 - DNA, 20 bp, Arachis_hypogaea
agggaggccgaggtcgttgt

SEQ ID NO: 1385 - DNA, 20 bp, Arachis_hypogaea
ttcgccgccgacaacgacct

SEQ ID NO: 1386 - DNA, 20 bp, Arachis_hypogaea
cggcagcgacagggaggccg

SEQ ID NO: 1387 - DNA, 20 bp, Arachis_hypogaea
agtagacggcagcgacaggg

SEQ ID NO: 1388 - DNA, 20 bp, Arachis_hypogaea
tgaagtagacggcagcgaca

SEQ ID NO: 1389 - DNA, 20 bp, Arachis_hypogaea
ttgaagtagacggcagcgac

SEQ ID NO: 1390 - DNA, 20 bp, Arachis_hypogaea
cctctgagcattgaagtaga

SEQ ID NO: 1391 - DNA, 20 bp, Arachis_hypogaea
ccgtctacttcaatgctcag

SEQ ID NO: 1392 - DNA, 20 bp, Arachis_hypogaea
cgtctacttcaatgctcaga

SEQ ID NO: 1393 - DNA, 20 bp, Arachis_hypogaea
ctcagagggaaaccgctgcc

SEQ ID NO: 1394 - DNA, 20 bp, Arachis_hypogaea
taattaacgcctcctggcag

SEQ ID NO: 1395 - DNA, 20 bp, Arachis_hypogaea
agagggaaaccgctgccagg

SEQ ID NO: 1396 - DNA, 20 bp, Arachis_hypogaea
attaattaattaacgcctcc

SEQ ID NO: 1397 - DNA, 20 bp, Arachis_hypogaea
agtagctagagatcccagtg

SEQ ID NO: 1398 - DNA, 20 bp, Arachis_hypogaea
gtagctagagatcccagtgt

SEQ ID NO: 1399 - DNA, 20 bp, Arachis_hypogaea
atttttcattcacccacact

SEQ ID NO: 1400 - DNA, 20 bp, Arachis_hypogaea
tatttttcattcacccacac

SEQ ID NO: 1401 - DNA, 20 bp, Arachis_hypogaea
ttacttgtattgcagaaaaa

SEQ ID NO: 1402 - DNA, 20 bp, Arachis_hypogaea
tacttgtattgcagaaaaaa

SEQ ID NO: 1403 - DNA, 20 bp, Arachis_hypogaea
tatgtatgtatgtatgtaga

SEQ ID NO: 1404 - DNA, 20 bp, Arachis_hypogaea
atgtatgtatgtatgtagaa

SEQ ID NO: 1405 - DNA, 20 bp, Arachis_hypogaea
gctgtgtgttttaatttcca

SEQ ID NO: 1406 - DNA, 20 bp, Arachis_hypogaea
acgacatggcgtatatacca

SEQ ID NO: 1407 - DNA, 20 bp, Arachis_hypogaea
tatgattagatgaaacgaca

SEQ ID NO: 1408 - DNA, 20 bp, Arachis_hypogaea
atcataataattactacttt

SEQ ID NO: 1409 - DNA, 2439 bp, Arachis hypogaea
aacaacaaacaaaattcacttcatattcttcttcttctctttcattctctttaacaatggcaaagatgtcatcttcagatcctttagttcttggaagagtggttggagatgtcatccactctttcaatccaagtgttcaaatgtctgtcacttacaacaccaaacaagtcttcaatggccatgagttcttcccttctgctcttaccactaggcctaaggttgccattgatggtggtgacatgaggactttttacacactggtaataatttcattattcatacatatatatatatattatttgttataatagaagagaggcaaaaaataattaataaataatattatggtagatattaaaaaaaaaataagggtgatcattgttgatgattgtgagtttgcttttttttttcctatttaattttacactagatcaattttacttacaagatttcactactatttaatttgttgatcctttttctctcccaaaaaagggggaaaaacaaaaccctaggggttagttcttacaaaatttggagctatttgagaaaaattaatgtatcctcttgtttcttttgtagatcatgacagatcctgatgttcctggcccaagtgatccttatctgagagaacatttgcactggtaacttgatatatatgattgaatttgaacctaggttttttattttttttttattttatttttttctagtaaaattaggtttaattatataactttcatgtaatacaaaagaaaaatgtctgtttcatcatatatactaaaagttgaccttcaaaaacaaattatgagaagagaaatagtgacaataatcttatgattaatacatgaactttggtaaagttatactaaacctttttttggctctcaaatctaactagtgtatttagaacttattagccttttcacttttgaatacaaattttcttaatttaattaaagcattagctatagataaatccccaatatgtttaatttatccactaaaaatagtgatatcacatgaactatcttataattaagaagaagcctttttcccctctcttacacacacactctcttttttttcctcctaatattcacattttaaacaaatactaatataattcttgtacaattattgtaaatacatatacttggtatattacctctaacatatatatctatttatagcaattatacaaaaaattatacacaataattaattatttatataacatttttgcacattttaatacacaaaaaataattaagtgtagatagagaggcttctgatctaacagtctactcttaattggttgcttttgcaggatggttacagacattcctggcacaacagatgccacatttggtaggtttcattgattcatagatctaattaaaatacaccatatatgtgttattctgtgttattcatttctatatataattcaaattgacctaccttattattattaagtgaaaatgttttatacattatatagactacccaactatcaaatgactatactaattttttttttatgtatctttcaaaaataaaatagttacaccaaatatttgcatgaactgaagtctagaatttagttcatattgtactatattatgaatacaattactaaaattatctactaataaaatgacaaagattcacatgtagttatttttatgtcaattatagttaatagttaaaaatcgttaaataataatttagttaaattaatggttcttaaatattaacatcatgtgagtttttaccggataaaatatatatatttatatataaatacatagtgactgattttaataattaatatatgtctgataagtaataaaaaagataaatgaataatagtaacaattttgtttgatgtaaaatggaagggaaagagatagtgagctatgaaagccctaagccacagatagggatacacaggtatgtgtttgtgttgttgaagcaaaagagaaggcagagtgtaaccccaccaagttcaagggatcacttcaacactatcaacttctctgctcactctgacctttctcttcctgttgctgctgtctacttcaatgctcagagagaaaccgccgctagaagacgctaattcatatatatgtagctgcatgcacacctatatatatatatatttattattagtaaataaagcaagaagcatgcgagcaaggttaggttatggctatatatcatcatcatcatcatgaagaatgaaaagggtatggcacgtggcttcttctcataggtggtgttaatcttggatatatagtttaggtgtcacttattattatatcatgtatgtggctaattaatgtattgtgttagttgtatgtgttgtttgtgagttatgtaatataatgttatgaacttgagaattaagaataaataaatatctttttattacta

SEQ ID NO: 1410 - protein, 174 aa, Arachis hypogaea
MAKMSSSDPLVLGRVVGDVIHSFNPSVQMSVTYNTKQVFNGHEFFPSALTTRPKVAIDGGDMRTFYTLIMTDPDVPGPSDPYLREHLHWMVTDIPGTTDATFGKEIVSYESPKPQIGIHRYVFVLLKQKRRQSVTPPSSRDHFNTINFSAHSDLSLPVAAVYFNAQRETAARRR

SEQ ID NO: 1411 - DNA, 20 bp, Arachis_hypogaea
tctttcattctctttaacaa

SEQ ID NO: 1412 - DNA, 20 bp, Arachis_hypogaea
accactcttccaagaactaa

SEQ ID NO: 1413 - DNA, 20 bp, Arachis_hypogaea
tcttcagatcctttagttct

SEQ ID NO: 1414 - DNA, 20 bp, Arachis_hypogaea
tcctttagttcttggaagag

SEQ ID NO: 1415 - DNA, 20 bp, Arachis_hypogaea
ttagttcttggaagagtggt

SEQ ID NO: 1416 - DNA, 20 bp, Arachis_hypogaea
gaacacttggattgaaagag

SEQ ID NO: 1417 - DNA, 20 bp, Arachis_hypogaea
gtgacagacatttgaacact

SEQ ID NO: 1418 - DNA, 20 bp, Arachis_hypogaea
atggccattgaagacttgtt

SEQ ID NO: 1419 - DNA, 20 bp, Arachis_hypogaea
aacaccaaacaagtcttcaa

SEQ ID NO: 1420 - DNA, 20 bp, Arachis_hypogaea
gagcagaagggaagaactca

SEQ ID NO: 1421 - DNA, 20 bp, Arachis_hypogaea
gcctagtggtaagagcagaa

SEQ ID NO: 1422 - DNA, 20 bp, Arachis_hypogaea
ggcctagtggtaagagcaga

SEQ ID NO: 1423 - DNA, 20 bp, Arachis_hypogaea
tcccttctgctcttaccact

SEQ ID NO: 1424 - DNA, 20 bp, Arachis_hypogaea
aatggcaaccttaggcctag

SEQ ID NO: 1425 - DNA, 20 bp, Arachis_hypogaea
tgctcttaccactaggccta

SEQ ID NO: 1426 - DNA, 20 bp, Arachis_hypogaea
ccaccatcaatggcaacctt

SEQ ID NO: 1427 - DNA, 20 bp, Arachis_hypogaea
aggcctaaggttgccattga

SEQ ID NO: 1428 - DNA, 20 bp, Arachis_hypogaea
cctcatgtcaccaccatcaa

SEQ ID NO: 1429 - DNA, 20 bp, Arachis_hypogaea
cctaaggttgccattgatgg

SEQ ID NO: 1430 - DNA, 20 bp, Arachis_hypogaea
ccattgatggtggtgacatg

SEQ ID NO: 1431 - DNA, 20 bp, Arachis_hypogaea
catgaggactttttacacac

SEQ ID NO: 1432 - DNA, 20 bp, Arachis_hypogaea
tatttgttataatagaagag

SEQ ID NO: 1433 - DNA, 20 bp, Arachis_hypogaea
ataattaataaataatatta

SEQ ID NO: 1434 - DNA, 20 bp, Arachis_hypogaea
tagatattaaaaaaaaaata

SEQ ID NO: 1435 - DNA, 20 bp, Arachis_hypogaea
agatattaaaaaaaaaataa

SEQ ID NO: 1436 - DNA, 20 bp, Arachis_hypogaea
gatctagtgtaaaattaaat

SEQ ID NO: 1437 - DNA, 20 bp, Arachis_hypogaea
cccttttttgggagagaaaa

SEQ ID NO: 1438 - DNA, 20 bp, Arachis_hypogaea
tcctttttctctcccaaaaa

SEQ ID NO: 1439 - DNA, 20 bp, Arachis_hypogaea
cctttttctctcccaaaaaa

SEQ ID NO: 1440 - DNA, 20 bp, Arachis_hypogaea
tttgtttttccccctttttt

SEQ ID NO: 1441 - DNA, 20 bp, Arachis_hypogaea
ctttttctctcccaaaaaag

SEQ ID NO: 1442 - DNA, 20 bp, Arachis_hypogaea
ttttgtttttcccccttttt

SEQ ID NO: 1443 - DNA, 20 bp, Arachis_hypogaea
tttttctctcccaaaaaagg

SEQ ID NO: 1444 - DNA, 20 bp, Arachis_hypogaea
agggggaaaaacaaaaccct

SEQ ID NO: 1445 - DNA, 20 bp, Arachis_hypogaea
gggggaaaaacaaaacccta

SEQ ID NO: 1446 - DNA, 20 bp, Arachis_hypogaea
ggggaaaaacaaaaccctag

SEQ ID NO: 1447 - DNA, 20 bp, Arachis_hypogaea
tttgtaagaactaaccccta

SEQ ID NO: 1448 - DNA, 20 bp, Arachis_hypogaea
ttttgtaagaactaacccct

SEQ ID NO: 1449 - DNA, 20 bp, Arachis_hypogaea
gggttagttcttacaaaatt

SEQ ID NO: 1450 - DNA, 20 bp, Arachis_hypogaea
tgatctacaaaagaaacaag

SEQ ID NO: 1451 - DNA, 20 bp, Arachis_hypogaea
tcacttgggccaggaacatc

SEQ ID NO: 1452 - DNA, 20 bp, Arachis_hypogaea
atgacagatcctgatgttcc

SEQ ID NO: 1453 - DNA, 20 bp, Arachis_hypogaea
agataaggatcacttgggcc

SEQ ID NO: 1454 - DNA, 20 bp, Arachis_hypogaea
ctctcagataaggatcactt

SEQ ID NO: 1455 - DNA, 20 bp, Arachis_hypogaea
tctctcagataaggatcact

SEQ ID NO: 1456 - DNA, 20 bp, Arachis_hypogaea
tgcaaatgttctctcagata

SEQ ID NO: 1457 - DNA, 20 bp, Arachis_hypogaea
atctgagagaacatttgcac

SEQ ID NO: 1458 - DNA, 20 bp, Arachis_hypogaea
atatgattgaatttgaacct

SEQ ID NO: 1459 - DNA, 20 bp, Arachis_hypogaea
aaaaaaaaaataaaaaacct

SEQ ID NO: 1460 - DNA, 20 bp, Arachis_hypogaea
tatttttttctagtaaaatt

SEQ ID NO: 1461 - DNA, 20 bp, Arachis_hypogaea
ttctcataatttgtttttga

SEQ ID NO: 1462 - DNA, 20 bp, Arachis_hypogaea
tatgattaatacatgaactt

SEQ ID NO: 1463 - DNA, 20 bp, Arachis_hypogaea
agttatactaaacctttttt

SEQ ID NO: 1464 - DNA, 20 bp, Arachis_hypogaea
tagatttgagagccaaaaaa

SEQ ID NO: 1465 - DNA, 20 bp, Arachis_hypogaea
atttgtattcaaaagtgaaa

SEQ ID NO: 1466 - DNA, 20 bp, Arachis_hypogaea
tggataaattaaacatattg

SEQ ID NO: 1467 - DNA, 20 bp, Arachis_hypogaea
gtggataaattaaacatatt

SEQ ID NO: 1468 - DNA, 20 bp, Arachis_hypogaea
agtggataaattaaacatat

SEQ ID NO: 1469 - DNA, 20 bp, Arachis_hypogaea
tgtgatatcactatttttag

SEQ ID NO: 1470 - DNA, 20 bp, Arachis_hypogaea
gtgtgtaagagaggggaaaa

SEQ ID NO: 1471 - DNA, 20 bp, Arachis_hypogaea
agagtgtgtgtgtaagagag

SEQ ID NO: 1472 - DNA, 20 bp, Arachis_hypogaea
gagagtgtgtgtgtaagaga

SEQ ID NO: 1473 - DNA, 20 bp, Arachis_hypogaea
agagagtgtgtgtgtaagag

SEQ ID NO: 1474 - DNA, 20 bp, Arachis_hypogaea
tttaaaatgtgaatattagg

SEQ ID NO: 1475 - DNA, 20 bp, Arachis_hypogaea
ttgtttaaaatgtgaatatt

SEQ ID NO: 1476 - DNA, 20 bp, Arachis_hypogaea
ttattgtaaatacatatact

SEQ ID NO: 1477 - DNA, 20 bp, Arachis_hypogaea
taaatagatatatatgttag

SEQ ID NO: 1478 - DNA, 20 bp, Arachis_hypogaea
ataattaagtgtagatagag

SEQ ID NO: 1479 - DNA, 20 bp, Arachis_hypogaea
tctaacagtctactcttaat

SEQ ID NO: 1480 - DNA, 20 bp, Arachis_hypogaea
tcttaattggttgcttttgc

SEQ ID NO: 1481 - DNA, 20 bp, Arachis_hypogaea
aattggttgcttttgcagga

SEQ ID NO: 1482 - DNA, 20 bp, Arachis_hypogaea
aggatggttacagacattcc

SEQ ID NO: 1483 - DNA, 20 bp, Arachis_hypogaea
aatgtggcatctgttgtgcc

SEQ ID NO: 1484 - DNA, 20 bp, Arachis_hypogaea
ggcacaacagatgccacatt

SEQ ID NO: 1485 - DNA, 20 bp, Arachis_hypogaea
tcaatgaaacctaccaaatg

SEQ ID NO: 1486 - DNA, 20 bp, Arachis_hypogaea
caacagatgccacatttggt

SEQ ID NO: 1487 - DNA, 20 bp, Arachis_hypogaea
aacacagaataacacatata

SEQ ID NO: 1488 - DNA, 20 bp, Arachis_hypogaea
tcacttaataataataaggt

SEQ ID NO: 1489 - DNA, 20 bp, Arachis_hypogaea
attttcacttaataataata

SEQ ID NO: 1490 - DNA, 20 bp, Arachis_hypogaea
agtatagtcatttgatagtt

SEQ ID NO: 1491 - DNA, 20 bp, Arachis_hypogaea
tagtatagtcatttgatagt

SEQ ID NO: 1492 - DNA, 20 bp, Arachis_hypogaea
cttcagttcatgcaaatatt

SEQ ID NO: 1493 - DNA, 20 bp, Arachis_hypogaea
taataatttagttaaattaa

SEQ ID NO: 1494 - DNA, 20 bp, Arachis_hypogaea
aacatcatgtgagtttttac

SEQ ID NO: 1495 - DNA, 20 bp, Arachis_hypogaea
ataaatatatatattttatc

SEQ ID NO: 1496 - DNA, 20 bp, Arachis_hypogaea
caattttgtttgatgtaaaa

SEQ ID NO: 1497 - DNA, 20 bp, Arachis_hypogaea
tttgtttgatgtaaaatgga

SEQ ID NO: 1498 - DNA, 20 bp, Arachis_hypogaea
ttgtttgatgtaaaatggaa

SEQ ID NO: 1499 - DNA, 20 bp, Arachis_hypogaea
gtatccctatctgtggctta

SEQ ID NO: 1500 - DNA, 20 bp, Arachis_hypogaea
tgtatccctatctgtggctt

SEQ ID NO: 1501 - DNA, 20 bp, Arachis_hypogaea
gaaagccctaagccacagat

SEQ ID NO: 1502 - DNA, 20 bp, Arachis_hypogaea
aaagccctaagccacagata

SEQ ID NO: 1503 - DNA, 20 bp, Arachis_hypogaea
tacctgtgtatccctatctg

SEQ ID NO: 1504 - DNA, 20 bp, Arachis_hypogaea
agccacagatagggatacac

SEQ ID NO: 1505 - DNA, 20 bp, Arachis_hypogaea
tgttgttgaagcaaaagaga

SEQ ID NO: 1506 - DNA, 20 bp, Arachis_hypogaea
gtgatcccttgaacttggtg

SEQ ID NO: 1507 - DNA, 20 bp, Arachis_hypogaea
agtgatcccttgaacttggt

SEQ ID NO: 1508 - DNA, 20 bp, Arachis_hypogaea
aagtgatcccttgaacttgg

SEQ ID NO: 1509 - DNA, 20 bp, Arachis_hypogaea
gtgtaaccccaccaagttca

SEQ ID NO: 1510 - DNA, 20 bp, Arachis_hypogaea
ttgaagtgatcccttgaact

SEQ ID NO: 1511 - DNA, 20 bp, Arachis_hypogaea
tgtaaccccaccaagttcaa

SEQ ID NO: 1512 - DNA, 20 bp, Arachis_hypogaea
cagcagcaacaggaagagaa

SEQ ID NO: 1513 - DNA, 20 bp, Arachis_hypogaea
ttgaagtagacagcagcaac

SEQ ID NO: 1514 - DNA, 20 bp, Arachis_hypogaea
gaattagcgtcttctagcgg

SEQ ID NO: 1515 - DNA, 20 bp, Arachis_hypogaea
tatgaattagcgtcttctag

SEQ ID NO: 1516 - DNA, 20 bp, Arachis_hypogaea
taataaatatatatatatat

SEQ ID NO: 1517 - DNA, 20 bp, Arachis_hypogaea
aagcaagaagcatgcgagca

SEQ ID NO: 1518 - DNA, 20 bp, Arachis_hypogaea
agaagcatgcgagcaaggtt

SEQ ID NO: 1519 - DNA, 20 bp, Arachis_hypogaea
atgcgagcaaggttaggtta

SEQ ID NO: 1520 - DNA, 20 bp, Arachis_hypogaea
catcatcatgaagaatgaaa

SEQ ID NO: 1521 - DNA, 20 bp, Arachis_hypogaea
atcatcatgaagaatgaaaa

SEQ ID NO: 1522 - DNA, 20 bp, Arachis_hypogaea
catgaagaatgaaaagggta

SEQ ID NO: 1523 - DNA, 20 bp, Arachis_hypogaea
aatgaaaagggtatggcacg

SEQ ID NO: 1524 - DNA, 20 bp, Arachis_hypogaea
ggcacgtggcttcttctcat

SEQ ID NO: 1525 - DNA, 20 bp, Arachis_hypogaea
acgtggcttcttctcatagg

SEQ ID NO: 1526 - DNA, 20 bp, Arachis_hypogaea
ctcataggtggtgttaatct

SEQ ID NO: 1527 - DNA, 20 bp, Arachis_hypogaea
taatcttggatatatagttt

SEQ ID NO: 1528 - DNA, 20 bp, Arachis_hypogaea
ttattattatatcatgtatg

SEQ ID NO: 1529 - DNA, 1689 bp, Arachis hypogaea
ctagcgtgcgtgtaagaaagaacacaaattacatacctaaaaaacacatgtcctttccttaaatagaaaagcccttagatctttgctccatggaatctttctttgtgctttatgtggctatatatatgcattgaggaataggatttggtttccaaggtattcacattatattaccaaagttttattaatatatttatatttgtggttctcttgtttctcaataataatgtctaggagtatgatggaaccacttgctgtgggaagagtgataggagatgtggttgatgtattcaacccaagtgtgagaatgaatgtgatttattccactaagcaagttgctaatggtcatgagcttatgccttcaacccttgtgcccaagcctagagttgagattggtggtcatgacttaagggatgcttataccttggtaattcaattaattaattaattaattaattattagttgctagctattcttattttaattttcatgttgatacttggtattttgtttggtagattatgacagacccagatgctccaagtcctagtgatccatacttgagggaacatcttcactggtttgtagtttgtacaaacaatcatattcattgattaattaattattattctttttagataaactaataattctctctaatgaacttaactggttccaattttccaggatggttgtagatattcctggcaccactgatgcatcttttggttagttcctctaactttctattttgttaggattgattcttgtacaagactttttctttaattagatgagactcacgtataattgtctttatgaaaatttaatatttaagcatcattagataatttagcatatttaattaatttttaattattaattttacatgaaaataactgtatgtgagttttgacgtatatttattagtgtaacaatgtatatatacatatcttattacatgttcttaactttgatgtacgtgaatgaattataaagggagagagatagtggagtatgagagtccaaggccaatgataggaatacacagatatgtgttcatattgttgaagcagcatagagggagacaaacagtgagtccccctgcttcgagagagcaattcaacacaagaatcttctcacaggaaaatggccttggcctacctgttgctgctgtttacttcaatgctcaaagagaaactgctgctaggagaaggtgattcattcattccattctgaatcaacaacaatctacctaaggtggtgcatagcaatgcacgtggaataaatcatattattatctaattgtgtcgtcttattcgttacctggtcagttattattacttcttctattctaagatattcgttactttttaaagtcgtgaatattttaagatggaagaaacattgtatggtagaataaggagatatgtgtttgatgaaatgaaacctttttctctattttttgttgttgtttggagtgcacaataattggtgggttttggaagataggagatattctagtagggatattcctgatgtactttacttgacttggtgtgctatgttacctattaggttgttgtgagaagttaaggaccaaattgatgatgttgttccttttaagaattttatgatttgtgatgggaaattaaaggacacatactataataacaaagctttatata

SEQ ID NO: 1530 - protein, 174 aa, Arachis hypogaea
MSRSMMEPLAVGRVIGDVVDVFNPSVRMNVIYSTKQVANGHELMPSTLVPKPRVEIGGHDLRDAYTLIMTDPDAPSPSDPYLREHLHWMVVDIPGTTDASFGREIVEYESPRPMIGIHRYVFILLKQHRGRQTVSPPASREQFNTRIFSQENGLGLPVAAVYFNAQRETAARRR

SEQ ID NO: 1531 - DNA, 20 bp, Arachis_hypogaea
ggaaaggacatgtgtttttt

SEQ ID NO: 1532 - DNA, 20 bp, Arachis_hypogaea
gggcttttctatttaaggaa

SEQ ID NO: 1533 - DNA, 20 bp, Arachis_hypogaea
tctaagggcttttctattta

SEQ ID NO: 1534 - DNA, 20 bp, Arachis_hypogaea
tccatggagcaaagatctaa

SEQ ID NO: 1535 - DNA, 20 bp, Arachis_hypogaea
ttccatggagcaaagatcta

SEQ ID NO: 1536 - DNA, 20 bp, Arachis_hypogaea
gcccttagatctttgctcca

SEQ ID NO: 1537 - DNA, 20 bp, Arachis_hypogaea
aagcacaaagaaagattcca

SEQ ID NO: 1538 - DNA, 20 bp, Arachis_hypogaea
atctttctttgtgctttatg

SEQ ID NO: 1539 - DNA, 20 bp, Arachis_hypogaea
gtggctatatatatgcattg

SEQ ID NO: 1540 - DNA, 20 bp, Arachis_hypogaea
atatatatgcattgaggaat

SEQ ID NO: 1541 - DNA, 20 bp, Arachis_hypogaea
atgcattgaggaataggatt

SEQ ID NO: 1542 - DNA, 20 bp, Arachis_hypogaea
ggaataggatttggtttcca

SEQ ID NO: 1543 - DNA, 20 bp, Arachis_hypogaea
gtaatataatgtgaatacct

SEQ ID NO: 1544 - DNA, 20 bp, Arachis_hypogaea
taaatatattaataaaactt

SEQ ID NO: 1545 - DNA, 20 bp, Arachis_hypogaea
tattaatatatttatatttg

SEQ ID NO: 1546 - DNA, 20 bp, Arachis_hypogaea
gtttctcaataataatgtct

SEQ ID NO: 1547 - DNA, 20 bp, Arachis_hypogaea
aataatgtctaggagtatga

SEQ ID NO: 1548 - DNA, 20 bp, Arachis_hypogaea
atcactcttcccacagcaag

SEQ ID NO: 1549 - DNA, 20 bp, Arachis_hypogaea
tatgatggaaccacttgctg

SEQ ID NO: 1550 - DNA, 20 bp, Arachis_hypogaea
atgatggaaccacttgctgt

SEQ ID NO: 1551 - DNA, 20 bp, Arachis_hypogaea
cttgctgtgggaagagtgat

SEQ ID NO: 1552 - DNA, 20 bp, Arachis_hypogaea
gggaagagtgataggagatg

SEQ ID NO: 1553 - DNA, 20 bp, Arachis_hypogaea
tcacattcattctcacactt

SEQ ID NO: 1554 - DNA, 20 bp, Arachis_hypogaea
atcacattcattctcacact

SEQ ID NO: 1555 - DNA, 20 bp, Arachis_hypogaea
accattagcaacttgcttag

SEQ ID NO: 1556 - DNA, 20 bp, Arachis_hypogaea
tccactaagcaagttgctaa

SEQ ID NO: 1557 - DNA, 20 bp, Arachis_hypogaea
ggcttgggcacaagggttga

SEQ ID NO: 1558 - DNA, 20 bp, Arachis_hypogaea
aactctaggcttgggcacaa

SEQ ID NO: 1559 - DNA, 20 bp, Arachis_hypogaea
caactctaggcttgggcaca

SEQ ID NO: 1560 - DNA, 20 bp, Arachis_hypogaea
ccaatctcaactctaggctt

SEQ ID NO: 1561 - DNA, 20 bp, Arachis_hypogaea
accaatctcaactctaggct

SEQ ID NO: 1562 - DNA, 20 bp, Arachis_hypogaea
tgaccaccaatctcaactct

SEQ ID NO: 1563 - DNA, 20 bp, Arachis_hypogaea
cccaagcctagagttgagat

SEQ ID NO: 1564 - DNA, 20 bp, Arachis_hypogaea
aagcctagagttgagattgg

SEQ ID NO: 1565 - DNA, 20 bp, Arachis_hypogaea
agattggtggtcatgactta

SEQ ID NO: 1566 - DNA, 20 bp, Arachis_hypogaea
gattggtggtcatgacttaa

SEQ ID NO: 1567 - DNA, 20 bp, Arachis_hypogaea
cttaagggatgcttatacct

SEQ ID NO: 1568 - DNA, 20 bp, Arachis_hypogaea
attaattaattgaattacca

SEQ ID NO: 1569 - DNA, 20 bp, Arachis_hypogaea
ttaattttcatgttgatact

SEQ ID NO: 1570 - DNA, 20 bp, Arachis_hypogaea
ttgatacttggtattttgtt

SEQ ID NO: 1571 - DNA, 20 bp, Arachis_hypogaea
cactaggacttggagcatct

SEQ ID NO: 1572 - DNA, 20 bp, Arachis_hypogaea
tcactaggacttggagcatc

SEQ ID NO: 1573 - DNA, 20 bp, Arachis_hypogaea
aagtatggatcactaggact

SEQ ID NO: 1574 - DNA, 20 bp, Arachis_hypogaea
tccctcaagtatggatcact

SEQ ID NO: 1575 - DNA, 20 bp, Arachis_hypogaea
gtcctagtgatccatacttg

SEQ ID NO: 1576 - DNA, 20 bp, Arachis_hypogaea
tgaagatgttccctcaagta

SEQ ID NO: 1577 - DNA, 20 bp, Arachis_hypogaea
tcctagtgatccatacttga

SEQ ID NO: 1578 - DNA, 20 bp, Arachis_hypogaea
acttgagggaacatcttcac

SEQ ID NO: 1579 - DNA, 20 bp, Arachis_hypogaea
ttctctctaatgaacttaac

SEQ ID NO: 1580 - DNA, 20 bp, Arachis_hypogaea
ctacaaccatcctggaaaat

SEQ ID NO: 1581 - DNA, 20 bp, Arachis_hypogaea
ttaactggttccaattttcc

SEQ ID NO: 1582 - DNA, 20 bp, Arachis_hypogaea
ctggttccaattttccagga

SEQ ID NO: 1583 - DNA, 20 bp, Arachis_hypogaea
aggaatatctacaaccatcc

SEQ ID NO: 1584 - DNA, 20 bp, Arachis_hypogaea
aggatggttgtagatattcc

SEQ ID NO: 1585 - DNA, 20 bp, Arachis_hypogaea
aaagatgcatcagtggtgcc

SEQ ID NO: 1586 - DNA, 20 bp, Arachis_hypogaea
ctaaccaaaagatgcatcag

SEQ ID NO: 1587 - DNA, 20 bp, Arachis_hypogaea
ggcaccactgatgcatcttt

SEQ ID NO: 1588 - DNA, 20 bp, Arachis_hypogaea
ctaacaaaatagaaagttag

SEQ ID NO: 1589 - DNA, 20 bp, Arachis_hypogaea
ctctaactttctattttgtt

SEQ ID NO: 1590 - DNA, 20 bp, Arachis_hypogaea
tgtacgtgaatgaattataa

SEQ ID NO: 1591 - DNA, 20 bp, Arachis_hypogaea
gtacgtgaatgaattataaa

SEQ ID NO: 1592 - DNA, 20 bp, Arachis_hypogaea
ttataaagggagagagatag

SEQ ID NO: 1593 - DNA, 20 bp, Arachis_hypogaea
tagtggagtatgagagtcca

SEQ ID NO: 1594 - DNA, 20 bp, Arachis_hypogaea
tgtattcctatcattggcct

SEQ ID NO: 1595 - DNA, 20 bp, Arachis_hypogaea
gagagtccaaggccaatgat

SEQ ID NO: 1596 - DNA, 20 bp, Arachis_hypogaea
tatctgtgtattcctatcat

SEQ ID NO: 1597 - DNA, 20 bp, Arachis_hypogaea
atattgttgaagcagcatag

SEQ ID NO: 1598 - DNA, 20 bp, Arachis_hypogaea
tattgttgaagcagcataga

SEQ ID NO: 1599 - DNA, 20 bp, Arachis_hypogaea
aattgctctctcgaagcagg

SEQ ID NO: 1600 - DNA, 20 bp, Arachis_hypogaea
gaattgctctctcgaagcag

SEQ ID NO: 1601 - DNA, 20 bp, Arachis_hypogaea
tgaattgctctctcgaagca

SEQ ID NO: 1602 - DNA, 20 bp, Arachis_hypogaea
ttgaattgctctctcgaagc

SEQ ID NO: 1603 - DNA, 20 bp, Arachis_hypogaea
caacacaagaatcttctcac

SEQ ID NO: 1604 - DNA, 20 bp, Arachis_hypogaea
agaatcttctcacaggaaaa

SEQ ID NO: 1605 - DNA, 20 bp, Arachis_hypogaea
ttctcacaggaaaatggcct

SEQ ID NO: 1606 - DNA, 20 bp, Arachis_hypogaea
cagcagcaacaggtaggcca

SEQ ID NO: 1607 - DNA, 20 bp, Arachis_hypogaea
agtaaacagcagcaacaggt

SEQ ID NO: 1608 - DNA, 20 bp, Arachis_hypogaea
ttgaagtaaacagcagcaac

SEQ ID NO: 1609 - DNA, 20 bp, Arachis_hypogaea
ctcaaagagaaactgctgct

SEQ ID NO: 1610 - DNA, 20 bp, Arachis_hypogaea
gagaaactgctgctaggaga

SEQ ID NO: 1611 - DNA, 20 bp, Arachis_hypogaea
agattgttgttgattcagaa

SEQ ID NO: 1612 - DNA, 20 bp, Arachis_hypogaea
aatcaacaacaatctaccta

SEQ ID NO: 1613 - DNA, 20 bp, Arachis_hypogaea
caacaacaatctacctaagg

SEQ ID NO: 1614 - DNA, 20 bp, Arachis_hypogaea
gcattgctatgcaccacctt

SEQ ID NO: 1615 - DNA, 20 bp, Arachis_hypogaea
gtggtgcatagcaatgcacg

SEQ ID NO: 1616 - DNA, 20 bp, Arachis_hypogaea
gtgtcgtcttattcgttacc

SEQ ID NO: 1617 - DNA, 20 bp, Arachis_hypogaea
agaagtaataataactgacc

SEQ ID NO: 1618 - DNA, 20 bp, Arachis_hypogaea
aagtcgtgaatattttaaga

SEQ ID NO: 1619 - DNA, 20 bp, Arachis_hypogaea
aagatggaagaaacattgta

SEQ ID NO: 1620 - DNA, 20 bp, Arachis_hypogaea
aaacattgtatggtagaata

SEQ ID NO: 1621 - DNA, 20 bp, Arachis_hypogaea
acaacaaaaaatagagaaaa

SEQ ID NO: 1622 - DNA, 20 bp, Arachis_hypogaea
ctctattttttgttgttgtt

SEQ ID NO: 1623 - DNA, 20 bp, Arachis_hypogaea
tgtttggagtgcacaataat

SEQ ID NO: 1624 - DNA, 20 bp, Arachis_hypogaea
ttggagtgcacaataattgg

SEQ ID NO: 1625 - DNA, 20 bp, Arachis_hypogaea
tggagtgcacaataattggt

SEQ ID NO: 1626 - DNA, 20 bp, Arachis_hypogaea
gcacaataattggtgggttt

SEQ ID NO: 1627 - DNA, 20 bp, Arachis_hypogaea
attggtgggttttggaagat

SEQ ID NO: 1628 - DNA, 20 bp, Arachis_hypogaea
aagataggagatattctagt

SEQ ID NO: 1629 - DNA, 20 bp, Arachis_hypogaea
agataggagatattctagta

SEQ ID NO: 1630 - DNA, 20 bp, Arachis_hypogaea
caagtcaagtaaagtacatc

SEQ ID NO: 1631 - DNA, 20 bp, Arachis_hypogaea
ctgatgtactttacttgact

SEQ ID NO: 1632 - DNA, 20 bp, Arachis_hypogaea
ggtgtgctatgttacctatt

SEQ ID NO: 1633 - DNA, 20 bp, Arachis_hypogaea
acttctcacaacaacctaat

SEQ ID NO: 1634 - DNA, 20 bp, Arachis_hypogaea
taggttgttgtgagaagtta

SEQ ID NO: 1635 - DNA, 20 bp, Arachis_hypogaea
aaggaacaacatcatcaatt

SEQ ID NO: 1636 - DNA, 20 bp, Arachis_hypogaea
caaatcataaaattcttaaa

SEQ ID NO: 1637 - DNA, 20 bp, Arachis_hypogaea
aagaattttatgatttgtga

SEQ ID NO: 1638 - DNA, 20 bp, Arachis_hypogaea
agaattttatgatttgtgat

SEQ ID NO: 1639 - DNA, 20 bp, Arachis_hypogaea
gatttgtgatgggaaattaa

SEQ ID NO: 1640 - DNA, 1638 bp, Arachis hypogaea
acatacctaaaaaacacatgtcctttccttaaatagaaaagcccttagatctttgttccatggaatctttctttgtgctttatctggctatatatatgcattgaggatttggtttccaaggtattcacattatatttatatttgtggttctcttgtttctcaataataatgtctaggaatatgatggaaccacttgctgtgggaagagtgataggagatgtggttgatgtattcaacccaagtgtgagaatgaatgtgatttattccactaagcaagttgctaatggtcatgagcttatgccttcaacccttgtgcccaagcctagagttgagattggtggtcatgacttaagggatgcttataccttggtaattcaattaattaattattacttagcttgctattcttattctaattttcatgttgatacttggtattttgtttggtagattatgacagacccagatgctccaagtcctagtgatccatacttgagggaacatcttcactggtttgtagtttgtacaaacaatcatattcattgatatgttatttattatttcttttagataaactgataattctctctaatgaacttaactggttccaattttccaggatggttgtagatattcctggcaccactgatgcatcttttggttagttcctctaataactttctattttgtcacgattgattcttgtgcaagactttttctttaattagatatgagagtcgcgtataattgtttttatgaaaattcaatatttaagcatctttagataatttagcatatttgattaatttttaattattaattttacatgaaaataattgcatgtgaatttttacgtatatttattaatgtaacaatgtatatacacatatcttattacatgttgttaactttgatgtatgtgaatgaattataaagggagagagatagtggagtatgagagtccaaggccagtgataggaatacacagatatgtgttcatattgttgaagcagcatagagggagacaaacagtgagtcccccggcttcgagagaacaattcaacacaagaatcttctctcaggaaaatggccttggcctacctgttgctgctgtttacttcaatgctcaaagagaaactgctgctaggagaaggtgattcattccttccattccgactcaacaacaatctacttaaggtggtgcatatatagcaatgcacgttgaataaatcatgttattatctaattgtgtcgtcgttacctggtcagttattattacttcttatattctaagatattcgttactttttaaagttgcaaatattttaaaatggaagaaacattgtatggtagaataaggggataggtgtttgatgaattgaaacatttttctttcttttttttgttgtttggagtgcacaataattggtgggttttggaagataggagatattctagtagggatattcttgatgtactttagtttacttgacttggtgtgctatgttacctattaggttgttgtgagaagttaaggaccaaattgaagatgttgttccttttaagaattttatgatttgtgatagaaagttaaaggacacgcactataataacaaagctttatatatatggcttgtc

SEQ ID NO: 1641 - protein, 174 aa, Arachis hypogaea
MSRNMMEPLAVGRVIGDVVDVFNPSVRMNVIYSTKQVANGHELMPSTLVPKPRVEIGGHDLRDAYTLIMTDPDAPSPSDPYLREHLHWMVVDIPGTTDASFGREIVEYESPRPVIGIHRYVFILLKQHRGRQTVSPPASREQFNTRIFSQENGLGLPVAAVYFNAQRETAARRR

SEQ ID NO: 1642 - DNA, 20 bp, Arachis_hypogaea
ggaaaggacatgtgtttttt

SEQ ID NO: 1643 - DNA, 20 bp, Arachis_hypogaea
gggcttttctatttaaggaa

SEQ ID NO: 1644 - DNA, 20 bp, Arachis_hypogaea
tctaagggcttttctattta

SEQ ID NO: 1645 - DNA, 20 bp, Arachis_hypogaea
tccatggaacaaagatctaa

SEQ ID NO: 1646 - DNA, 20 bp, Arachis_hypogaea
ttccatggaacaaagatcta

SEQ ID NO: 1647 - DNA, 20 bp, Arachis_hypogaea
gcccttagatctttgttcca

SEQ ID NO: 1648 - DNA, 20 bp, Arachis_hypogaea
aagcacaaagaaagattcca

SEQ ID NO: 1649 - DNA, 20 bp, Arachis_hypogaea
atctttctttgtgctttatc

SEQ ID NO: 1650 - DNA, 20 bp, Arachis_hypogaea
ctggctatatatatgcattg

SEQ ID NO: 1651 - DNA, 20 bp, Arachis_hypogaea
atatatatgcattgaggatt

SEQ ID NO: 1652 - DNA, 20 bp, Arachis_hypogaea
cattgaggatttggtttcca

SEQ ID NO: 1653 - DNA, 20 bp, Arachis_hypogaea
taaatataatgtgaatacct

SEQ ID NO: 1654 - DNA, 20 bp, Arachis_hypogaea
tcacattatatttatatttg

SEQ ID NO: 1655 - DNA, 20 bp, Arachis_hypogaea
gtttctcaataataatgtct

SEQ ID NO: 1656 - DNA, 20 bp, Arachis_hypogaea
aataatgtctaggaatatga

SEQ ID NO: 1657 - DNA, 20 bp, Arachis_hypogaea
atcactcttcccacagcaag

SEQ ID NO: 1658 - DNA, 20 bp, Arachis_hypogaea
tatgatggaaccacttgctg

SEQ ID NO: 1659 - DNA, 20 bp, Arachis_hypogaea
atgatggaaccacttgctgt

SEQ ID NO: 1660 - DNA, 20 bp, Arachis_hypogaea
cttgctgtgggaagagtgat

SEQ ID NO: 1661 - DNA, 20 bp, Arachis_hypogaea
gggaagagtgataggagatg

SEQ ID NO: 1662 - DNA, 20 bp, Arachis_hypogaea
tcacattcattctcacactt

SEQ ID NO: 1663 - DNA, 20 bp, Arachis_hypogaea
atcacattcattctcacact

SEQ ID NO: 1664 - DNA, 20 bp, Arachis_hypogaea
accattagcaacttgcttag

SEQ ID NO: 1665 - DNA, 20 bp, Arachis_hypogaea
tccactaagcaagttgctaa

SEQ ID NO: 1666 - DNA, 20 bp, Arachis_hypogaea
ggcttgggcacaagggttga

SEQ ID NO: 1667 - DNA, 20 bp, Arachis_hypogaea
aactctaggcttgggcacaa

SEQ ID NO: 1668 - DNA, 20 bp, Arachis_hypogaea
caactctaggcttgggcaca

SEQ ID NO: 1669 - DNA, 20 bp, Arachis_hypogaea
ccaatctcaactctaggctt

SEQ ID NO: 1670 - DNA, 20 bp, Arachis_hypogaea
accaatctcaactctaggct

SEQ ID NO: 1671 - DNA, 20 bp, Arachis_hypogaea
tgaccaccaatctcaactct

SEQ ID NO: 1672 - DNA, 20 bp, Arachis_hypogaea
cccaagcctagagttgagat

SEQ ID NO: 1673 - DNA, 20 bp, Arachis_hypogaea
aagcctagagttgagattgg

SEQ ID NO: 1674 - DNA, 20 bp, Arachis_hypogaea
agattggtggtcatgactta

SEQ ID NO: 1675 - DNA, 20 bp, Arachis_hypogaea
gattggtggtcatgacttaa

SEQ ID NO: 1676 - DNA, 20 bp, Arachis_hypogaea
cttaagggatgcttatacct

SEQ ID NO: 1677 - DNA, 20 bp, Arachis_hypogaea
attaattaattgaattacca

SEQ ID NO: 1678 - DNA, 20 bp, Arachis_hypogaea
ctaattttcatgttgatact

SEQ ID NO: 1679 - DNA, 20 bp, Arachis_hypogaea
ttgatacttggtattttgtt

SEQ ID NO: 1680 - DNA, 20 bp, Arachis_hypogaea
cactaggacttggagcatct

SEQ ID NO: 1681 - DNA, 20 bp, Arachis_hypogaea
tcactaggacttggagcatc

SEQ ID NO: 1682 - DNA, 20 bp, Arachis_hypogaea
aagtatggatcactaggact

SEQ ID NO: 1683 - DNA, 20 bp, Arachis_hypogaea
tccctcaagtatggatcact

SEQ ID NO: 1684 - DNA, 20 bp, Arachis_hypogaea
gtcctagtgatccatacttg

SEQ ID NO: 1685 - DNA, 20 bp, Arachis_hypogaea
tgaagatgttccctcaagta

SEQ ID NO: 1686 - DNA, 20 bp, Arachis_hypogaea
tcctagtgatccatacttga

SEQ ID NO: 1687 - DNA, 20 bp, Arachis_hypogaea
acttgagggaacatcttcac

SEQ ID NO: 1688 - DNA, 20 bp, Arachis_hypogaea
ttctctctaatgaacttaac

SEQ ID NO: 1689 - DNA, 20 bp, Arachis_hypogaea
ctacaaccatcctggaaaat

SEQ ID NO: 1690 - DNA, 20 bp, Arachis_hypogaea
ttaactggttccaattttcc

SEQ ID NO: 1691 - DNA, 20 bp, Arachis_hypogaea
ctggttccaattttccagga

SEQ ID NO: 1692 - DNA, 20 bp, Arachis_hypogaea
aggaatatctacaaccatcc

SEQ ID NO: 1693 - DNA, 20 bp, Arachis_hypogaea
aggatggttgtagatattcc

SEQ ID NO: 1694 - DNA, 20 bp, Arachis_hypogaea
aaagatgcatcagtggtgcc

SEQ ID NO: 1695 - DNA, 20 bp, Arachis_hypogaea
ctaaccaaaagatgcatcag

SEQ ID NO: 1696 - DNA, 20 bp, Arachis_hypogaea
ggcaccactgatgcatcttt

SEQ ID NO: 1697 - DNA, 20 bp, Arachis_hypogaea
acaaaatagaaagttattag

SEQ ID NO: 1698 - DNA, 20 bp, Arachis_hypogaea
tgtatgtgaatgaattataa

SEQ ID NO: 1699 - DNA, 20 bp, Arachis_hypogaea
gtatgtgaatgaattataaa

SEQ ID NO: 1700 - DNA, 20 bp, Arachis_hypogaea
ttataaagggagagagatag

SEQ ID NO: 1701 - DNA, 20 bp, Arachis_hypogaea
tagtggagtatgagagtcca

SEQ ID NO: 1702 - DNA, 20 bp, Arachis_hypogaea
tgtattcctatcactggcct

SEQ ID NO: 1703 - DNA, 20 bp, Arachis_hypogaea
gagagtccaaggccagtgat

SEQ ID NO: 1704 - DNA, 20 bp, Arachis_hypogaea
tatctgtgtattcctatcac

SEQ ID NO: 1705 - DNA, 20 bp, Arachis_hypogaea
atattgttgaagcagcatag

SEQ ID NO: 1706 - DNA, 20 bp, Arachis_hypogaea
tattgttgaagcagcataga

SEQ ID NO: 1707 - DNA, 20 bp, Arachis_hypogaea
gagacaaacagtgagtcccc

SEQ ID NO: 1708 - DNA, 20 bp, Arachis_hypogaea
aattgttctctcgaagccgg

SEQ ID NO: 1709 - DNA, 20 bp, Arachis_hypogaea
gaattgttctctcgaagccg

SEQ ID NO: 1710 - DNA, 20 bp, Arachis_hypogaea
tgaattgttctctcgaagcc

SEQ ID NO: 1711 - DNA, 20 bp, Arachis_hypogaea
ttgaattgttctctcgaagc

SEQ ID NO: 1712 - DNA, 20 bp, Arachis_hypogaea
caacacaagaatcttctctc

SEQ ID NO: 1713 - DNA, 20 bp, Arachis_hypogaea
agaatcttctctcaggaaaa

SEQ ID NO: 1714 - DNA, 20 bp, Arachis_hypogaea
ttctctcaggaaaatggcct

SEQ ID NO: 1715 - DNA, 20 bp, Arachis_hypogaea
cagcagcaacaggtaggcca

SEQ ID NO: 1716 - DNA, 20 bp, Arachis_hypogaea
agtaaacagcagcaacaggt

SEQ ID NO: 1717 - DNA, 20 bp, Arachis_hypogaea
ttgaagtaaacagcagcaac

SEQ ID NO: 1718 - DNA, 20 bp, Arachis_hypogaea
ctcaaagagaaactgctgct

SEQ ID NO: 1719 - DNA, 20 bp, Arachis_hypogaea
gagaaactgctgctaggaga

SEQ ID NO: 1720 - DNA, 20 bp, Arachis_hypogaea
tgttgttgagtcggaatgga

SEQ ID NO: 1721 - DNA, 20 bp, Arachis_hypogaea
agattgttgttgagtcggaa

SEQ ID NO: 1722 - DNA, 20 bp, Arachis_hypogaea
taagtagattgttgttgagt

SEQ ID NO: 1723 - DNA, 20 bp, Arachis_hypogaea
actcaacaacaatctactta

SEQ ID NO: 1724 - DNA, 20 bp, Arachis_hypogaea
caacaacaatctacttaagg

SEQ ID NO: 1725 - DNA, 20 bp, Arachis_hypogaea
ctaattgtgtcgtcgttacc

SEQ ID NO: 1726 - DNA, 20 bp, Arachis_hypogaea
agaagtaataataactgacc

SEQ ID NO: 1727 - DNA, 20 bp, Arachis_hypogaea
aagttgcaaatattttaaaa

SEQ ID NO: 1728 - DNA, 20 bp, Arachis_hypogaea
aaaatggaagaaacattgta

SEQ ID NO: 1729 - DNA, 20 bp, Arachis_hypogaea
aaacattgtatggtagaata

SEQ ID NO: 1730 - DNA, 20 bp, Arachis_hypogaea
aacattgtatggtagaataa

SEQ ID NO: 1731 - DNA, 20 bp, Arachis_hypogaea
acattgtatggtagaataag

SEQ ID NO: 1732 - DNA, 20 bp, Arachis_hypogaea
gtatggtagaataaggggat

SEQ ID NO: 1733 - DNA, 20 bp, Arachis_hypogaea
tctttcttttttttgttgtt

SEQ ID NO: 1734 - DNA, 20 bp, Arachis_hypogaea
tgtttggagtgcacaataat

SEQ ID NO: 1735 - DNA, 20 bp, Arachis_hypogaea
ttggagtgcacaataattgg

SEQ ID NO: 1736 - DNA, 20 bp, Arachis_hypogaea
tggagtgcacaataattggt

SEQ ID NO: 1737 - DNA, 20 bp, Arachis_hypogaea
gcacaataattggtgggttt

SEQ ID NO: 1738 - DNA, 20 bp, Arachis_hypogaea
attggtgggttttggaagat

SEQ ID NO: 1739 - DNA, 20 bp, Arachis_hypogaea
aagataggagatattctagt

SEQ ID NO: 1740 - DNA, 20 bp, Arachis_hypogaea
agataggagatattctagta

SEQ ID NO: 1741 - DNA, 20 bp, Arachis_hypogaea
gtactttagtttacttgact

SEQ ID NO: 1742 - DNA, 20 bp, Arachis_hypogaea
ggtgtgctatgttacctatt

SEQ ID NO: 1743 - DNA, 20 bp, Arachis_hypogaea
acttctcacaacaacctaat

SEQ ID NO: 1744 - DNA, 20 bp, Arachis_hypogaea
taggttgttgtgagaagtta

SEQ ID NO: 1745 - DNA, 20 bp, Arachis_hypogaea
aaggaacaacatcttcaatt

SEQ ID NO: 1746 - DNA, 20 bp, Arachis_hypogaea
caaatcataaaattcttaaa

SEQ ID NO: 1747 - DNA, 20 bp, Arachis_hypogaea
gatttgtgatagaaagttaa

SEQ ID NO: 1748 - DNA, 20 bp, Arachis_hypogaea
ataacaaagctttatatata

SEQ ID NO: 1749 - DNA, 3658 bp, Arachis hypogaea
cataaataggggataagccctctcacgttttagtaaaatttgacaacagacagttaaacaaactgacgtaaacatatatagctgtatgcctagaacaacaataatggaccctcttgctgttggaggggtaatcggagatgttgttgatccttttgtaagttgtgtttctctgaggattgtatatcacaataactcagaagttgttaatagtttggagttgaagccatcccaaattatcaatcaaccaagagttgacgttggtggtgatgattttagaactttttacactctggtacgtagaatttactactttgtagttttctgttattaccgtatattttaaaatttcatacaatgaataactatatctgtatattatgatgcagataatggtggaccctgatgcacctagtcctactaatccaaatatgagagagtatttacactggtgagtccatagttctgtatctccatctgtgtctgctatagttacatgactgaattctgtgagtactgtttccttataaatggttaaaatttgagggtcttttatttaaataaaaaaattattattttattgcgatgcaaaaatatattattttatgcatttagttgtgttttacagttaaaaaatacggaaatagaagaatacatttttgtattaagagtatgcaaaaatcagaaaaaaattttttgtattaatataattttttattttcgtattctttacctgtgaaaacacagcttaatgtacaaaatgatatattctgtgtctgcgtttaaatcggtaaataatgaattttttatttaaataaaaaacttaaaatttgttagcctaaaaactgtagtataacgttgaccgtgcttttcttgtatgtaacagtgtaactcacatcttacttgaatttttgaaagtgtctaattataataaaaaaattacatgacccaaaataaatagtcacattcaatatatgtatgatatagttgaagttaatatttaagtgaaaacctattaaatttaagggatcgtttgatttgtatttgtatattttatttttaaaattttataaagaaaatagagataaaaaatataaattttattgttttcaccaaaatataataataaaatttgttagctttaaataagaagtctctattatttaaaaaattttaaaatattattaattgatattccatttaagtaatattattaaaaaatgtttgaattttaaaataaaaaagaataatctcagtttgagagtataaattttagaaaattcaaaattcttttagcaagtaacatgaaaattcaaaatttatattaaaattaaaaatgaactaatctcatcactttatactataccctaaaataatttacaattttttaactattctagacttcattaacatgtgaaaaaagtttcctggtaatggaaacagaagtgatatatgtaagctaatgataaaatattaaggtacagtttaggcaaatagtttaattaaattttttttttgaaaaaatagtttaaataataaatacttatattaaaaataatttataaataaattattttgtatttaattttttagttttaaaagtgcttatttaaaaaaattgtgataacaaattttttattatgagagaagttattttttttaaattttcttataagtacttaaatagttttttttagaaagttataatttagttttgaaaattgtatcagacattaatagtctaagtgttgttcgcagtgcggtttggatcggttttgagaccaaaactcatatgatccaaacactaattttacttgcggtgcgatttagattggatgattttttctttttaaataatcgatctaatttcaagcggttttgattagattggatttgcgattttgtaaattaaacaaattaaatacatataacaaatctcaacatcaatttttaaataatcaacaatgacataacaagtctcaatattatcttaaaaaaccaacgataacataacaatacaaataaaattataggttagttaaaataaataaataaataacatattaaacataaaatatttattaaataagaataatacatatataatataaaaatgtataacaaattgatcatgctataaatataattttaaatataataataaactaataattatagcacattaaacggtttggattggattgaatcagttatgaaaagtagatccaaaatccgatccgatccagcagtttacaaaaatagaattcaatcaaatctaaattagtgcggttttaatcgatttttggtttgaattgaattgaatgaacaatttaaggggcggagctagaataaacattagaggggggctaaaaatatttacacaataaaataatattaaaataattttttgaggaggctaaattgaaatttacatataatttacatgtaaacatttaaaattaggggccgttgccccctttcgcagtatgtagctccacctctggtttaaatttgaacagttctaactaatactattactttttataagttaaaaattcaaaaaataacttttaaaacttttcaaacggcctaagattatctaagaatataatacacacattactcattgtagcatgacatatttttttgaagccatgacaatgcaatcaacatgtatcccatagtaatacgttctctaactatgtatcatatcctaattaataactcaaaaatatctctatatatcactgatgagttttgcttcgatcttaaaccatggtttaggttggtaatgaatattccagggacgacaggaacgagcttcggtaagcagtcaatcaattaacatatcattgttatgtagaataatgtgattattaaataaacgaaaatttaactaagtgaaaggtaacaaatgcaggacaagagatagttggatacgaaagcccaagaccaatagcaggaatacataggatggtgtttgtgttgtacaaacaaatgaggaggcaagaagtgatacaccctccaagatggcgtaacaacttcaacactcgtgacttcgcacaagtctacaatcttggaactcctgttgctgctctctacttcaattgccagcgggagagcggttggggtggaaggagaatcatcatttagctttatccttccttcaacccaaaaatgggcttaggcccacccttaatttaccaaaatgggcttttttaattcataagtgttaaaacactactcatttaaaacctaaacccatcaccaaaatttgacctctatccttcaacaaaaataaaaaaactactattattttctctaccgttgttatgacccaaaactatataataataatactcaaaagggtctgaaagttgtactagttattgaattaaacaaggcactgatgaagcccttgatcaatggcagcagcacctgcctcactaggagctgtacctaggttcaaattctcgatgagaaatataaaacccatattagtagtaatagtgtatgtgaacgaatgtgtgttgtgtaatgtataagaaccaaaaaataaaaaacaaggcagtgatgatttgtgtcaaatataagtttgagctgtatcttatatttttttaaggttaatattagaatttactttctaaataacgaattgaag

SEQ ID NO: 1750 - protein, 180 aa, Arachis hypogaea
MPRTTIMDPLAVGGVIGDVVDPFVSCVSLRIVYHNNSEVVNSLELKPSQIINQPRVDVGGDDFRTFYTLIMVDPDAPSPTNPNMREYLHWLVMNIPGTTGTSFGQEIVGYESPRPIAGIHRMVFVLYKQMRRQEVIHPPRWRNNFNTRDFAQVYNLGTPVAALYFNCQRESGWGGRRIII

SEQ ID NO: 1751 - DNA, 20 bp, Arachis_hypogaea
aattttactaaaacgtgaga

SEQ ID NO: 1752 - DNA, 20 bp, Arachis_hypogaea
aaattttactaaaacgtgag

SEQ ID NO: 1753 - DNA, 20 bp, Arachis_hypogaea
gggtccattattgttgttct

SEQ ID NO: 1754 - DNA, 20 bp, Arachis_hypogaea
tatgcctagaacaacaataa

SEQ ID NO: 1755 - DNA, 20 bp, Arachis_hypogaea
ttacccctccaacagcaaga

SEQ ID NO: 1756 - DNA, 20 bp, Arachis_hypogaea
attacccctccaacagcaag

SEQ ID NO: 1757 - DNA, 20 bp, Arachis_hypogaea
ataatggaccctcttgctgt

SEQ ID NO: 1758 - DNA, 20 bp, Arachis_hypogaea
atggaccctcttgctgttgg

SEQ ID NO: 1759 - DNA, 20 bp, Arachis_hypogaea
tggaccctcttgctgttgga

SEQ ID NO: 1760 - DNA, 20 bp, Arachis_hypogaea
ggaccctcttgctgttggag

SEQ ID NO: 1761 - DNA, 20 bp, Arachis_hypogaea
cttgctgttggaggggtaat

SEQ ID NO: 1762 - DNA, 20 bp, Arachis_hypogaea
agagaaacacaacttacaaa

SEQ ID NO: 1763 - DNA, 20 bp, Arachis_hypogaea
ttgtaagttgtgtttctctg

SEQ ID NO: 1764 - DNA, 20 bp, Arachis_hypogaea
ctcagaagttgttaatagtt

SEQ ID NO: 1765 - DNA, 20 bp, Arachis_hypogaea
ggttgattgataatttggga

SEQ ID NO: 1766 - DNA, 20 bp, Arachis_hypogaea
tcttggttgattgataattt

SEQ ID NO: 1767 - DNA, 20 bp, Arachis_hypogaea
ctcttggttgattgataatt

SEQ ID NO: 1768 - DNA, 20 bp, Arachis_hypogaea
tcaccaccaacgtcaactct

SEQ ID NO: 1769 - DNA, 20 bp, Arachis_hypogaea
aatcaaccaagagttgacgt

SEQ ID NO: 1770 - DNA, 20 bp, Arachis_hypogaea
caaccaagagttgacgttgg

SEQ ID NO: 1771 - DNA, 20 bp, Arachis_hypogaea
ttttagaactttttacactc

SEQ ID NO: 1772 - DNA, 20 bp, Arachis_hypogaea
gtatgaaattttaaaatata

SEQ ID NO: 1773 - DNA, 20 bp, Arachis_hypogaea
gtatattatgatgcagataa

SEQ ID NO: 1774 - DNA, 20 bp, Arachis_hypogaea
tattatgatgcagataatgg

SEQ ID NO: 1775 - DNA, 20 bp, Arachis_hypogaea
tagtaggactaggtgcatca

SEQ ID NO: 1776 - DNA, 20 bp, Arachis_hypogaea
ttagtaggactaggtgcatc

SEQ ID NO: 1777 - DNA, 20 bp, Arachis_hypogaea
atatttggattagtaggact

SEQ ID NO: 1778 - DNA, 20 bp, Arachis_hypogaea
tctctcatatttggattagt

SEQ ID NO: 1779 - DNA, 20 bp, Arachis_hypogaea
tgtaaatactctctcatatt

SEQ ID NO: 1780 - DNA, 20 bp, Arachis_hypogaea
atatgagagagtatttacac

SEQ ID NO: 1781 - DNA, 20 bp, Arachis_hypogaea
cagatggagatacagaacta

SEQ ID NO: 1782 - DNA, 20 bp, Arachis_hypogaea
gtaactatagcagacacaga

SEQ ID NO: 1783 - DNA, 20 bp, Arachis_hypogaea
gagtactgtttccttataaa

SEQ ID NO: 1784 - DNA, 20 bp, Arachis_hypogaea
tcaaattttaaccatttata

SEQ ID NO: 1785 - DNA, 20 bp, Arachis_hypogaea
ttataaatggttaaaatttg

SEQ ID NO: 1786 - DNA, 20 bp, Arachis_hypogaea
tataaatggttaaaatttga

SEQ ID NO: 1787 - DNA, 20 bp, Arachis_hypogaea
tgttttacagttaaaaaata

SEQ ID NO: 1788 - DNA, 20 bp, Arachis_hypogaea
acattaagctgtgttttcac

SEQ ID NO: 1789 - DNA, 20 bp, Arachis_hypogaea
ttctgtgtctgcgtttaaat

SEQ ID NO: 1790 - DNA, 20 bp, Arachis_hypogaea
aacgttatactacagttttt

SEQ ID NO: 1791 - DNA, 20 bp, Arachis_hypogaea
tgttacatacaagaaaagca

SEQ ID NO: 1792 - DNA, 20 bp, Arachis_hypogaea
tgaatgtgactatttatttt

SEQ ID NO: 1793 - DNA, 20 bp, Arachis_hypogaea
ttgaatgtgactatttattt

SEQ ID NO: 1794 - DNA, 20 bp, Arachis_hypogaea
aacgatcccttaaatttaat

SEQ ID NO: 1795 - DNA, 20 bp, Arachis_hypogaea
gtgaaaacctattaaattta

SEQ ID NO: 1796 - DNA, 20 bp, Arachis_hypogaea
tgaaaacctattaaatttaa

SEQ ID NO: 1797 - DNA, 20 bp, Arachis_hypogaea
caaattttattattatattt

SEQ ID NO: 1798 - DNA, 20 bp, Arachis_hypogaea
ttttaataatattacttaaa

SEQ ID NO: 1799 - DNA, 20 bp, Arachis_hypogaea
aaaaattgtaaattatttta

SEQ ID NO: 1800 - DNA, 20 bp, Arachis_hypogaea
aaaaaattgtaaattatttt

SEQ ID NO: 1801 - DNA, 20 bp, Arachis_hypogaea
aacatgtgaaaaaagtttcc

SEQ ID NO: 1802 - DNA, 20 bp, Arachis_hypogaea
tgaaaaaagtttcctggtaa

SEQ ID NO: 1803 - DNA, 20 bp, Arachis_hypogaea
tcacttctgtttccattacc

SEQ ID NO: 1804 - DNA, 20 bp, Arachis_hypogaea
aagctaatgataaaatatta

SEQ ID NO: 1805 - DNA, 20 bp, Arachis_hypogaea
aaaatattaaggtacagttt

SEQ ID NO: 1806 - DNA, 20 bp, Arachis_hypogaea
tctaagtgttgttcgcagtg

SEQ ID NO: 1807 - DNA, 20 bp, Arachis_hypogaea
gtgttgttcgcagtgcggtt

SEQ ID NO: 1808 - DNA, 20 bp, Arachis_hypogaea
gttcgcagtgcggtttggat

SEQ ID NO: 1809 - DNA, 20 bp, Arachis_hypogaea
tgtttggatcatatgagttt

SEQ ID NO: 1810 - DNA, 20 bp, Arachis_hypogaea
ccgcaagtaaaattagtgtt

SEQ ID NO: 1811 - DNA, 20 bp, Arachis_hypogaea
ccaaacactaattttacttg

SEQ ID NO: 1812 - DNA, 20 bp, Arachis_hypogaea
acttgcggtgcgatttagat

SEQ ID NO: 1813 - DNA, 20 bp, Arachis_hypogaea
ataatcgatctaatttcaag

SEQ ID NO: 1814 - DNA, 20 bp, Arachis_hypogaea
tcaagcggttttgattagat

SEQ ID NO: 1815 - DNA, 20 bp, Arachis_hypogaea
ttgtattgttatgttatcgt

SEQ ID NO: 1816 - DNA, 20 bp, Arachis_hypogaea
aacaatacaaataaaattat

SEQ ID NO: 1817 - DNA, 20 bp, Arachis_hypogaea
aataattatagcacattaaa

SEQ ID NO: 1818 - DNA, 20 bp, Arachis_hypogaea
ttatagcacattaaacggtt

SEQ ID NO: 1819 - DNA, 20 bp, Arachis_hypogaea
gcacattaaacggtttggat

SEQ ID NO: 1820 - DNA, 20 bp, Arachis_hypogaea
tgctggatcggatcggattt

SEQ ID NO: 1821 - DNA, 20 bp, Arachis_hypogaea
tgtaaactgctggatcggat

SEQ ID NO: 1822 - DNA, 20 bp, Arachis_hypogaea
atttttgtaaactgctggat

SEQ ID NO: 1823 - DNA, 20 bp, Arachis_hypogaea
attctatttttgtaaactgc

SEQ ID NO: 1824 - DNA, 20 bp, Arachis_hypogaea
caatcaaatctaaattagtg

SEQ ID NO: 1825 - DNA, 20 bp, Arachis_hypogaea
gtgcggttttaatcgatttt

SEQ ID NO: 1826 - DNA, 20 bp, Arachis_hypogaea
tgaattgaatgaacaattta

SEQ ID NO: 1827 - DNA, 20 bp, Arachis_hypogaea
gaattgaatgaacaatttaa

SEQ ID NO: 1828 - DNA, 20 bp, Arachis_hypogaea
aattgaatgaacaatttaag

SEQ ID NO: 1829 - DNA, 20 bp, Arachis_hypogaea
tgaatgaacaatttaagggg

SEQ ID NO: 1830 - DNA, 20 bp, Arachis_hypogaea
ggagctagaataaacattag

SEQ ID NO: 1831 - DNA, 20 bp, Arachis_hypogaea
gagctagaataaacattaga

SEQ ID NO: 1832 - DNA, 20 bp, Arachis_hypogaea
agctagaataaacattagag

SEQ ID NO: 1833 - DNA, 20 bp, Arachis_hypogaea
gctagaataaacattagagg

SEQ ID NO: 1834 - DNA, 20 bp, Arachis_hypogaea
ctagaataaacattagaggg

SEQ ID NO: 1835 - DNA, 20 bp, Arachis_hypogaea
aatattaaaataattttttg

SEQ ID NO: 1836 - DNA, 20 bp, Arachis_hypogaea
attaaaataattttttgagg

SEQ ID NO: 1837 - DNA, 20 bp, Arachis_hypogaea
acatgtaaacatttaaaatt

SEQ ID NO: 1838 - DNA, 20 bp, Arachis_hypogaea
catgtaaacatttaaaatta

SEQ ID NO: 1839 - DNA, 20 bp, Arachis_hypogaea
atgtaaacatttaaaattag

SEQ ID NO: 1840 - DNA, 20 bp, Arachis_hypogaea
catactgcgaaagggggcaa

SEQ ID NO: 1841 - DNA, 20 bp, Arachis_hypogaea
gagctacatactgcgaaagg

SEQ ID NO: 1842 - DNA, 20 bp, Arachis_hypogaea
ggagctacatactgcgaaag

SEQ ID NO: 1843 - DNA, 20 bp, Arachis_hypogaea
tggagctacatactgcgaaa

SEQ ID NO: 1844 - DNA, 20 bp, Arachis_hypogaea
gtggagctacatactgcgaa

SEQ ID NO: 1845 - DNA, 20 bp, Arachis_hypogaea
gcagtatgtagctccacctc

SEQ ID NO: 1846 - DNA, 20 bp, Arachis_hypogaea
gttcaaatttaaaccagagg

SEQ ID NO: 1847 - DNA, 20 bp, Arachis_hypogaea
actgttcaaatttaaaccag

SEQ ID NO: 1848 - DNA, 20 bp, Arachis_hypogaea
aacttttaaaacttttcaaa

SEQ ID NO: 1849 - DNA, 20 bp, Arachis_hypogaea
ttatattcttagataatctt

SEQ ID NO: 1850 - DNA, 20 bp, Arachis_hypogaea
acatgttgattgcattgtca

SEQ ID NO: 1851 - DNA, 20 bp, Arachis_hypogaea
gttagagaacgtattactat

SEQ ID NO: 1852 - DNA, 20 bp, Arachis_hypogaea
agttagagaacgtattacta

SEQ ID NO: 1853 - DNA, 20 bp, Arachis_hypogaea
atatttttgagttattaatt

SEQ ID NO: 1854 - DNA, 20 bp, Arachis_hypogaea
tttgcttcgatcttaaacca

SEQ ID NO: 1855 - DNA, 20 bp, Arachis_hypogaea
tcgatcttaaaccatggttt

SEQ ID NO: 1856 - DNA, 20 bp, Arachis_hypogaea
ttcattaccaacctaaacca

SEQ ID NO: 1857 - DNA, 20 bp, Arachis_hypogaea
tcttaaaccatggtttaggt

SEQ ID NO: 1858 - DNA, 20 bp, Arachis_hypogaea
aggttggtaatgaatattcc

SEQ ID NO: 1859 - DNA, 20 bp, Arachis_hypogaea
ggttggtaatgaatattcca

SEQ ID NO: 1860 - DNA, 20 bp, Arachis_hypogaea
aagctcgttcctgtcgtccc

SEQ ID NO: 1861 - DNA, 20 bp, Arachis_hypogaea
atgaatattccagggacgac

SEQ ID NO: 1862 - DNA, 20 bp, Arachis_hypogaea
gggacgacaggaacgagctt

SEQ ID NO: 1863 - DNA, 20 bp, Arachis_hypogaea
cgaaaatttaactaagtgaa

SEQ ID NO: 1864 - DNA, 20 bp, Arachis_hypogaea
aagtgaaaggtaacaaatgc

SEQ ID NO: 1865 - DNA, 20 bp, Arachis_hypogaea
aatgcaggacaagagatagt

SEQ ID NO: 1866 - DNA, 20 bp, Arachis_hypogaea
gtattcctgctattggtctt

SEQ ID NO: 1867 - DNA, 20 bp, Arachis_hypogaea
tgtattcctgctattggtct

SEQ ID NO: 1868 - DNA, 20 bp, Arachis_hypogaea
gaaagcccaagaccaatagc

SEQ ID NO: 1869 - DNA, 20 bp, Arachis_hypogaea
atcctatgtattcctgctat

SEQ ID NO: 1870 - DNA, 20 bp, Arachis_hypogaea
gaccaatagcaggaatacat

SEQ ID NO: 1871 - DNA, 20 bp, Arachis_hypogaea
aatagcaggaatacatagga

SEQ ID NO: 1872 - DNA, 20 bp, Arachis_hypogaea
ttgtgttgtacaaacaaatg

SEQ ID NO: 1873 - DNA, 20 bp, Arachis_hypogaea
tgttgtacaaacaaatgagg

SEQ ID NO: 1874 - DNA, 20 bp, Arachis_hypogaea
agttgttacgccatcttgga

SEQ ID NO: 1875 - DNA, 20 bp, Arachis_hypogaea
aagtgatacaccctccaaga

SEQ ID NO: 1876 - DNA, 20 bp, Arachis_hypogaea
aagttgttacgccatcttgg

SEQ ID NO: 1877 - DNA, 20 bp, Arachis_hypogaea
ttgaagttgttacgccatct

SEQ ID NO: 1878 - DNA, 20 bp, Arachis_hypogaea
ttcgcacaagtctacaatct

SEQ ID NO: 1879 - DNA, 20 bp, Arachis_hypogaea
ttgaagtagagagcagcaac

SEQ ID NO: 1880 - DNA, 20 bp, Arachis_hypogaea
ctctctacttcaattgccag

SEQ ID NO: 1881 - DNA, 20 bp, Arachis_hypogaea
tctctacttcaattgccagc

SEQ ID NO: 1882 - DNA, 20 bp, Arachis_hypogaea
caccccaaccgctctcccgc

SEQ ID NO: 1883 - DNA, 20 bp, Arachis_hypogaea
ttcaattgccagcgggagag

SEQ ID NO: 1884 - DNA, 20 bp, Arachis_hypogaea
attgccagcgggagagcggt

SEQ ID NO: 1885 - DNA, 20 bp, Arachis_hypogaea
ttgccagcgggagagcggtt

SEQ ID NO: 1886 - DNA, 20 bp, Arachis_hypogaea
tgccagcgggagagcggttg

SEQ ID NO: 1887 - DNA, 20 bp, Arachis_hypogaea
cagcgggagagcggttgggg

SEQ ID NO: 1888 - DNA, 20 bp, Arachis_hypogaea
gggagagcggttggggtgga

SEQ ID NO: 1889 - DNA, 20 bp, Arachis_hypogaea
cccatttttgggttgaagga

SEQ ID NO: 1890 - DNA, 20 bp, Arachis_hypogaea
taagcccatttttgggttga

SEQ ID NO: 1891 - DNA, 20 bp, Arachis_hypogaea
tccttccttcaacccaaaaa

SEQ ID NO: 1892 - DNA, 20 bp, Arachis_hypogaea
ccttccttcaacccaaaaat

SEQ ID NO: 1893 - DNA, 20 bp, Arachis_hypogaea
gtgggcctaagcccattttt

SEQ ID NO: 1894 - DNA, 20 bp, Arachis_hypogaea
ggtgggcctaagcccatttt

SEQ ID NO: 1895 - DNA, 20 bp, Arachis_hypogaea
ttcaacccaaaaatgggctt

SEQ ID NO: 1896 - DNA, 20 bp, Arachis_hypogaea
cattttggtaaattaagggt

SEQ ID NO: 1897 - DNA, 20 bp, Arachis_hypogaea
ccattttggtaaattaaggg

SEQ ID NO: 1898 - DNA, 20 bp, Arachis_hypogaea
agcccattttggtaaattaa

SEQ ID NO: 1899 - DNA, 20 bp, Arachis_hypogaea
aagcccattttggtaaatta

SEQ ID NO: 1900 - DNA, 20 bp, Arachis_hypogaea
ccacccttaatttaccaaaa

SEQ ID NO: 1901 - DNA, 20 bp, Arachis_hypogaea
cacccttaatttaccaaaat

SEQ ID NO: 1902 - DNA, 20 bp, Arachis_hypogaea
tgaattaaaaaagcccattt

SEQ ID NO: 1903 - DNA, 20 bp, Arachis_hypogaea
caaattttggtgatgggttt

SEQ ID NO: 1904 - DNA, 20 bp, Arachis_hypogaea
agaggtcaaattttggtgat

SEQ ID NO: 1905 - DNA, 20 bp, Arachis_hypogaea
tagaggtcaaattttggtga

SEQ ID NO: 1906 - DNA, 20 bp, Arachis_hypogaea
gaaggatagaggtcaaattt

SEQ ID NO: 1907 - DNA, 20 bp, Arachis_hypogaea
ttatttttgttgaaggatag

SEQ ID NO: 1908 - DNA, 20 bp, Arachis_hypogaea
agtttttttatttttgttga

SEQ ID NO: 1909 - DNA, 20 bp, Arachis_hypogaea
atagttttgggtcataacaa

SEQ ID NO: 1910 - DNA, 20 bp, Arachis_hypogaea
tattattattatatagtttt

SEQ ID NO: 1911 - DNA, 20 bp, Arachis_hypogaea
gtattattattatatagttt

SEQ ID NO: 1912 - DNA, 20 bp, Arachis_hypogaea
atataataataatactcaaa

SEQ ID NO: 1913 - DNA, 20 bp, Arachis_hypogaea
tataataataatactcaaaa

SEQ ID NO: 1914 - DNA, 20 bp, Arachis_hypogaea
actagttattgaattaaaca

SEQ ID NO: 1915 - DNA, 20 bp, Arachis_hypogaea
gtgctgctgccattgatcaa

SEQ ID NO: 1916 - DNA, 20 bp, Arachis_hypogaea
ggtgctgctgccattgatca

SEQ ID NO: 1917 - DNA, 20 bp, Arachis_hypogaea
ctgatgaagcccttgatcaa

SEQ ID NO: 1918 - DNA, 20 bp, Arachis_hypogaea
ggtacagctcctagtgaggc

SEQ ID NO: 1919 - DNA, 20 bp, Arachis_hypogaea
gcagcagcacctgcctcact

SEQ ID NO: 1920 - DNA, 20 bp, Arachis_hypogaea
cctaggtacagctcctagtg

SEQ ID NO: 1921 - DNA, 20 bp, Arachis_hypogaea
cctcactaggagctgtacct

SEQ ID NO: 1922 - DNA, 20 bp, Arachis_hypogaea
ctcatcgagaatttgaacct

SEQ ID NO: 1923 - DNA, 20 bp, Arachis_hypogaea
tacactattactactaatat

SEQ ID NO: 1924 - DNA, 20 bp, Arachis_hypogaea
atacactattactactaata

SEQ ID NO: 1925 - DNA, 20 bp, Arachis_hypogaea
ctgccttgttttttattttt

SEQ ID NO: 1926 - DNA, 20 bp, Arachis_hypogaea
gaaccaaaaaataaaaaaca

SEQ ID NO: 1927 - DNA, 20 bp, Arachis_hypogaea
ctgtatcttatattttttta

SEQ ID NO: 1928 - DNA, 3287 bp, Arachis hypogaea
atggaccctcttactcttggaaggttgttaggggatgttgttgatccctttacaagttctgtatctttgaggattgcgtataacaataactcagaagtagtcaattgttctgacttcaagccctcccaaattaccaaacaaccaagagttgatgttggtggtgatgattttaggactttttacactctggtaagtatctctatatatgcacttcttatctttgaaaagatcatctttattactaattttaagcttacgtggcatgatgatgatatatatgtgtatatatatgtatatgcagattatggtagaccctgatgcacctagtcctactaatcccaatatgagggagtatttgcattggtatgttatgggtcaccatcttataacgtggatgaatatgtaacatatgtgttcctctatacacttctttactgctctaaaagtcgaatatacataaaggttggcttaattatctaaatatcaaatagtttattattatgagaaatattagaggattattagaatttattattttttattattagctagttattaatatttaaaagtataggataaagtatgttattggattattatataaaaaaaattagactaaaaaaattaaattaatagttaaattatgataaaaataataaattctaatgattctctagcatttgtttattattattaaaaaattttaagatatttgtctattagttaaattaaaattaattaatttttttaactaatagacaaatatcctattattgttattattattatttattattattatagctagagagagtaactcaattataagagaataataaatactatctaaagatgagattaactgtgactaattaacatataaggttaagtgtcatacataaaaaattaaataagatataaaaaatatattttactagtaagtatcgtaaaaataattattaaaattagtattagtagaatttttaagttatttcttctttattaaatatattacaaatatattaaaaaaatctaagaaatcaattacataacaagtcaactaagttaacttttttttattttaaaatttgaaaaactaaggtaacaggaagttgtttttattattattattattattattattattattattattattattattattattattattattattattattattattattttgtaataaacctatttttcaactgattagaattggtttcatttctaattaattgtattctttactaaacagcctgcattacttagcatcacaccaaactaattatttgaactttgaagtgatgccaattaacatatctatatatagatcagataccataccctaacgtgtccgtgttccggttcgttttcctttatgttttcggattttgttattgtggtgaacatttaagaaaaactgaaaaagtttaacaaatagagactacaacagagataaaatattacaaaaggtaaatatatatttcaatttttgcacttttaaatttatacggttatgtactctttttctttaatagattttgtcccacaaaattttactaatagaagattttcaacgagccacgagctacgtaagacttattttatttttgttggagtctgcttgtcggttgttttggtatcatttttttttatttctcggttaattagagcatctccaatgagactgattttgaagtttaagttttaaatatgacatatacatgaagtaaaataattttaaattttattttatattaaaataataaatctcatattcaatctcaattactatttatgatatataattttacaaatatcaatataacattttgtagaatttaaaataaaatacatataaaactaaattggaaatgctcttataatccttctgtacgttgaaacttgtatttcaacagctatatatccaaaaaaagatatcataccctaaccaaaaaattattctctgtccaaaagcgtatatgattcatgagatatattaaaacataaaactatatattatataacattatttactactttataaaaataaagatggaagtaaaaaataacagatgaatatataattaacatccagaagcaaaatagtagaaacaatttagagaggtattgattgtgttataaatgatgaggaacaaatgtataaatttgatatctgtgaaagatttatatattgatatatctcttaaatgactgctcaggttggtaattaacattccaggaactacaggggcgagcttcggtaagcacatatatatatacatatatggctcagatcggtaaacagtttaattaattttaaaaaaaatagtttaaataataaatatttatattaaaaataatttataaataaattattttatatttaattttttaatttaaaaaatatttattttaaaagaaatataataaaaaaatttattataaaaaatattttttttcaattttttataaatatttaaataattttttaaaaaattataatttattttaaaaattataccaaacattaatattattactttttataaattaaaaattcaaaaaataatttttaaaatttttgaaacggtctataagatttatgtaaaatgttaaagtacagtataacatattatatttatccatgttatatatatatattaaaattagttattataatataaaatatatataaaaatataaaatatatattaaaataagttaaattataattatatttatatataaatatatattaattaatttaaatagttaattttgatgttcaaataatatttttgtgtgatattatatatgttaagtaagaaaatataatgcagggcaagagataatgaagtatgagagtccagaaccaacggcaggaatacatagaatagtgtttgtgctgtacaagcaaatggtggggaggcaactgatacacgctccgacctggcgtaacaactttaacactcgcgactttgctcaagtttacaatctcggatctcctgttgctgctgtgtacttcaattgccagcgggagagtggttgtggtggaaggagaatgatctagcgtgaaagtgatagtataatactgatctaatagatataggcaaataaattgataagtacttcaatttaatacatatgtatgtgtgtgtttttttgttaagaattagctaaatcttgatataaaagaataattaaataagtgggaagttgtctgctgggctttgttaattcctcctatgtatgcaatgtatgcttcttaa

SEQ ID NO: 1929 - protein, 173 aa, Arachis hypogaea
MDPLTLGRLLGDVVDPFTSSVSLRIAYNNNSEVVNCSDFKPSQITKQPRVDVGGDDFRTFYTLIMVDPDAPSPTNPNMREYLHWLVINIPGTTGASFGQEIMKYESPEPTAGIHRIVFVLYKQMVGRQLIHAPTWRNNFNTRDFAQVYNLGSPVAAVYFNCQRESGCGGRRMI

SEQ ID NO: 1930 - DNA, 20 bp, Arachis_hypogaea
acaaccttccaagagtaaga

SEQ ID NO: 1931 - DNA, 20 bp, Arachis_hypogaea
aacaaccttccaagagtaag

SEQ ID NO: 1932 - DNA, 20 bp, Arachis_hypogaea
tggaccctcttactcttgga

SEQ ID NO: 1933 - DNA, 20 bp, Arachis_hypogaea
cttactcttggaaggttgtt

SEQ ID NO: 1934 - DNA, 20 bp, Arachis_hypogaea
ttactcttggaaggttgtta

SEQ ID NO: 1935 - DNA, 20 bp, Arachis_hypogaea
tactcttggaaggttgttag

SEQ ID NO: 1936 - DNA, 20 bp, Arachis_hypogaea
aaagatacagaacttgtaaa

SEQ ID NO: 1937 - DNA, 20 bp, Arachis_hypogaea
caaagatacagaacttgtaa

SEQ ID NO: 1938 - DNA, 20 bp, Arachis_hypogaea
ttacaagttctgtatctttg

SEQ ID NO: 1939 - DNA, 20 bp, Arachis_hypogaea
ggttgtttggtaatttggga

SEQ ID NO: 1940 - DNA, 20 bp, Arachis_hypogaea
tggttgtttggtaatttggg

SEQ ID NO: 1941 - DNA, 20 bp, Arachis_hypogaea
tcttggttgtttggtaattt

SEQ ID NO: 1942 - DNA, 20 bp, Arachis_hypogaea
ctcttggttgtttggtaatt

SEQ ID NO: 1943 - DNA, 20 bp, Arachis_hypogaea
aacatcaactcttggttgtt

SEQ ID NO: 1944 - DNA, 20 bp, Arachis_hypogaea
tcaccaccaacatcaactct

SEQ ID NO: 1945 - DNA, 20 bp, Arachis_hypogaea
aaacaaccaagagttgatgt

SEQ ID NO: 1946 - DNA, 20 bp, Arachis_hypogaea
caaccaagagttgatgttgg

SEQ ID NO: 1947 - DNA, 20 bp, Arachis_hypogaea
atgttggtggtgatgatttt

SEQ ID NO: 1948 - DNA, 20 bp, Arachis_hypogaea
ttttaggactttttacactc

SEQ ID NO: 1949 - DNA, 20 bp, Arachis_hypogaea
ttactaattttaagcttacg

SEQ ID NO: 1950 - DNA, 20 bp, Arachis_hypogaea
atatatgtatatgcagatta

SEQ ID NO: 1951 - DNA, 20 bp, Arachis_hypogaea
tagtaggactaggtgcatca

SEQ ID NO: 1952 - DNA, 20 bp, Arachis_hypogaea
ttagtaggactaggtgcatc

SEQ ID NO: 1953 - DNA, 20 bp, Arachis_hypogaea
atattgggattagtaggact

SEQ ID NO: 1954 - DNA, 20 bp, Arachis_hypogaea
tccctcatattgggattagt

SEQ ID NO: 1955 - DNA, 20 bp, Arachis_hypogaea
gtcctactaatcccaatatg

SEQ ID NO: 1956 - DNA, 20 bp, Arachis_hypogaea
tgcaaatactccctcatatt

SEQ ID NO: 1957 - DNA, 20 bp, Arachis_hypogaea
tcctactaatcccaatatga

SEQ ID NO: 1958 - DNA, 20 bp, Arachis_hypogaea
atgcaaatactccctcatat

SEQ ID NO: 1959 - DNA, 20 bp, Arachis_hypogaea
atatgagggagtatttgcat

SEQ ID NO: 1960 - DNA, 20 bp, Arachis_hypogaea
gtatttgcattggtatgtta

SEQ ID NO: 1961 - DNA, 20 bp, Arachis_hypogaea
tatttgcattggtatgttat

SEQ ID NO: 1962 - DNA, 20 bp, Arachis_hypogaea
tattcatccacgttataaga

SEQ ID NO: 1963 - DNA, 20 bp, Arachis_hypogaea
tgggtcaccatcttataacg

SEQ ID NO: 1964 - DNA, 20 bp, Arachis_hypogaea
agcagtaaagaagtgtatag

SEQ ID NO: 1965 - DNA, 20 bp, Arachis_hypogaea
taaaagtcgaatatacataa

SEQ ID NO: 1966 - DNA, 20 bp, Arachis_hypogaea
agtcgaatatacataaaggt

SEQ ID NO: 1967 - DNA, 20 bp, Arachis_hypogaea
attattatgagaaatattag

SEQ ID NO: 1968 - DNA, 20 bp, Arachis_hypogaea
ttattaatatttaaaagtat

SEQ ID NO: 1969 - DNA, 20 bp, Arachis_hypogaea
tataggataaagtatgttat

SEQ ID NO: 1970 - DNA, 20 bp, Arachis_hypogaea
ataataataataacaataat

SEQ ID NO: 1971 - DNA, 20 bp, Arachis_hypogaea
ctgtgactaattaacatata

SEQ ID NO: 1972 - DNA, 20 bp, Arachis_hypogaea
ttttaaaatttgaaaaacta

SEQ ID NO: 1973 - DNA, 20 bp, Arachis_hypogaea
atttgaaaaactaaggtaac

SEQ ID NO: 1974 - DNA, 20 bp, Arachis_hypogaea
attctaatcagttgaaaaat

SEQ ID NO: 1975 - DNA, 20 bp, Arachis_hypogaea
atttttcaactgattagaat

SEQ ID NO: 1976 - DNA, 20 bp, Arachis_hypogaea
ggtgtgatgctaagtaatgc

SEQ ID NO: 1977 - DNA, 20 bp, Arachis_hypogaea
caaagttcaaataattagtt

SEQ ID NO: 1978 - DNA, 20 bp, Arachis_hypogaea
ctatatatagatatgttaat

SEQ ID NO: 1979 - DNA, 20 bp, Arachis_hypogaea
aacacggacacgttagggta

SEQ ID NO: 1980 - DNA, 20 bp, Arachis_hypogaea
accggaacacggacacgtta

SEQ ID NO: 1981 - DNA, 20 bp, Arachis_hypogaea
aaccggaacacggacacgtt

SEQ ID NO: 1982 - DNA, 20 bp, Arachis_hypogaea
accctaacgtgtccgtgttc

SEQ ID NO: 1983 - DNA, 20 bp, Arachis_hypogaea
aaggaaaacgaaccggaaca

SEQ ID NO: 1984 - DNA, 20 bp, Arachis_hypogaea
aaacataaaggaaaacgaac

SEQ ID NO: 1985 - DNA, 20 bp, Arachis_hypogaea
aacaaaatccgaaaacataa

SEQ ID NO: 1986 - DNA, 20 bp, Arachis_hypogaea
ttcgttttcctttatgtttt

SEQ ID NO: 1987 - DNA, 20 bp, Arachis_hypogaea
gttttcggattttgttattg

SEQ ID NO: 1988 - DNA, 20 bp, Arachis_hypogaea
cagagataaaatattacaaa

SEQ ID NO: 1989 - DNA, 20 bp, Arachis_hypogaea
tttgcacttttaaatttata

SEQ ID NO: 1990 - DNA, 20 bp, Arachis_hypogaea
ttctattagtaaaattttgt

SEQ ID NO: 1991 - DNA, 20 bp, Arachis_hypogaea
cttctattagtaaaattttg

SEQ ID NO: 1992 - DNA, 20 bp, Arachis_hypogaea
aataagtcttacgtagctcg

SEQ ID NO: 1993 - DNA, 20 bp, Arachis_hypogaea
aagacttattttatttttgt

SEQ ID NO: 1994 - DNA, 20 bp, Arachis_hypogaea
ttttgttggagtctgcttgt

SEQ ID NO: 1995 - DNA, 20 bp, Arachis_hypogaea
agtctgcttgtcggttgttt

SEQ ID NO: 1996 - DNA, 20 bp, Arachis_hypogaea
tatcatttttttttatttct

SEQ ID NO: 1997 - DNA, 20 bp, Arachis_hypogaea
aacttcaaaatcagtctcat

SEQ ID NO: 1998 - DNA, 20 bp, Arachis_hypogaea
aaatacatataaaactaaat

SEQ ID NO: 1999 - DNA, 20 bp, Arachis_hypogaea
tacaagtttcaacgtacaga

SEQ ID NO: 2000 - DNA, 20 bp, Arachis_hypogaea
tagggtatgatatctttttt

SEQ ID NO: 2001 - DNA, 20 bp, Arachis_hypogaea
agagaataattttttggtta

SEQ ID NO: 2002 - DNA, 20 bp, Arachis_hypogaea
cagagaataattttttggtt

SEQ ID NO: 2003 - DNA, 20 bp, Arachis_hypogaea
ttggacagagaataattttt

SEQ ID NO: 2004 - DNA, 20 bp, Arachis_hypogaea
tcatgaatcatatacgcttt

SEQ ID NO: 2005 - DNA, 20 bp, Arachis_hypogaea
ctactttataaaaataaaga

SEQ ID NO: 2006 - DNA, 20 bp, Arachis_hypogaea
tgtttctactattttgcttc

SEQ ID NO: 2007 - DNA, 20 bp, Arachis_hypogaea
atagtagaaacaatttagag

SEQ ID NO: 2008 - DNA, 20 bp, Arachis_hypogaea
tgattgtgttataaatgatg

SEQ ID NO: 2009 - DNA, 20 bp, Arachis_hypogaea
tatctcttaaatgactgctc

SEQ ID NO: 2010 - DNA, 20 bp, Arachis_hypogaea
tcttaaatgactgctcaggt

SEQ ID NO: 2011 - DNA, 20 bp, Arachis_hypogaea
aggttggtaattaacattcc

SEQ ID NO: 2012 - DNA, 20 bp, Arachis_hypogaea
aagctcgcccctgtagttcc

SEQ ID NO: 2013 - DNA, 20 bp, Arachis_hypogaea
attaacattccaggaactac

SEQ ID NO: 2014 - DNA, 20 bp, Arachis_hypogaea
ttaacattccaggaactaca

SEQ ID NO: 2015 - DNA, 20 bp, Arachis_hypogaea
taacattccaggaactacag

SEQ ID NO: 2016 - DNA, 20 bp, Arachis_hypogaea
ggaactacaggggcgagctt

SEQ ID NO: 2017 - DNA, 20 bp, Arachis_hypogaea
cacatatatatatacatata

SEQ ID NO: 2018 - DNA, 20 bp, Arachis_hypogaea
tatacatatatggctcagat

SEQ ID NO: 2019 - DNA, 20 bp, Arachis_hypogaea
aaagtaataatattaatgtt

SEQ ID NO: 2020 - DNA, 20 bp, Arachis_hypogaea
aatttttaaaatttttgaaa

SEQ ID NO: 2021 - DNA, 20 bp, Arachis_hypogaea
tttaatatatatatataaca

SEQ ID NO: 2022 - DNA, 20 bp, Arachis_hypogaea
taagtaagaaaatataatgc

SEQ ID NO: 2023 - DNA, 20 bp, Arachis_hypogaea
aagtaagaaaatataatgca

SEQ ID NO: 2024 - DNA, 20 bp, Arachis_hypogaea
tgtattcctgccgttggttc

SEQ ID NO: 2025 - DNA, 20 bp, Arachis_hypogaea
gtatgagagtccagaaccaa

SEQ ID NO: 2026 - DNA, 20 bp, Arachis_hypogaea
gagagtccagaaccaacggc

SEQ ID NO: 2027 - DNA, 20 bp, Arachis_hypogaea
attctatgtattcctgccgt

SEQ ID NO: 2028 - DNA, 20 bp, Arachis_hypogaea
gtttgtgctgtacaagcaaa

SEQ ID NO: 2029 - DNA, 20 bp, Arachis_hypogaea
tgtgctgtacaagcaaatgg

SEQ ID NO: 2030 - DNA, 20 bp, Arachis_hypogaea
gtgctgtacaagcaaatggt

SEQ ID NO: 2031 - DNA, 20 bp, Arachis_hypogaea
tgctgtacaagcaaatggtg

SEQ ID NO: 2032 - DNA, 20 bp, Arachis_hypogaea
tgtacaagcaaatggtgggg

SEQ ID NO: 2033 - DNA, 20 bp, Arachis_hypogaea
aactgatacacgctccgacc

SEQ ID NO: 2034 - DNA, 20 bp, Arachis_hypogaea
ttaaagttgttacgccaggt

SEQ ID NO: 2035 - DNA, 20 bp, Arachis_hypogaea
agtgttaaagttgttacgcc

SEQ ID NO: 2036 - DNA, 20 bp, Arachis_hypogaea
tttgctcaagtttacaatct

SEQ ID NO: 2037 - DNA, 20 bp, Arachis_hypogaea
ttgaagtacacagcagcaac

SEQ ID NO: 2038 - DNA, 20 bp, Arachis_hypogaea
ctgtgtacttcaattgccag

SEQ ID NO: 2039 - DNA, 20 bp, Arachis_hypogaea
tgtgtacttcaattgccagc

SEQ ID NO: 2040 - DNA, 20 bp, Arachis_hypogaea
caccacaaccactctcccgc

SEQ ID NO: 2041 - DNA, 20 bp, Arachis_hypogaea
ttcaattgccagcgggagag

SEQ ID NO: 2042 - DNA, 20 bp, Arachis_hypogaea
tgccagcgggagagtggttg

SEQ ID NO: 2043 - DNA, 20 bp, Arachis_hypogaea
cagcgggagagtggttgtgg

SEQ ID NO: 2044 - DNA, 20 bp, Arachis_hypogaea
gggagagtggttgtggtgga

SEQ ID NO: 2045 - DNA, 20 bp, Arachis_hypogaea
aatactgatctaatagatat

SEQ ID NO: 2046 - DNA, 20 bp, Arachis_hypogaea
taaaagaataattaaataag

SEQ ID NO: 2047 - DNA, 20 bp, Arachis_hypogaea
aaaagaataattaaataagt

SEQ ID NO: 2048 - DNA, 20 bp, Arachis_hypogaea
ataagtgggaagttgtctgc

SEQ ID NO: 2049 - DNA, 20 bp, Arachis_hypogaea
taagtgggaagttgtctgct

SEQ ID NO: 2050 - DNA, 20 bp, Arachis_hypogaea
gcatacattgcatacatagg

SEQ ID NO: 2051 - DNA, 20 bp, Arachis_hypogaea
gaagcatacattgcatacat